(19) United States
(12) Patent Application Publication
(10) Pub. No.: US 2026/0297172 A1
(43) Pub. Date: Oct. 1, 2026

(54) ANTIBODIES TARGETING S100 PROTEINS AND USES THEREOF

(71) Applicant: ALBERT EINSTEIN COLLEGE OF MEDICINE, Bronx, NY (US)

(72) Inventors: Amit Verma, Bronx, NY (US); Rongbao Zhao, Bronx, NY (US); Srinivas Aluri, Bronx, NY (US)

(21) Appl. No.: 19/481,206

(22) PCT Filed: May 10, 2024
(86) PCT No.: PCT/US2024/028779

§ 371 Date: Nov. 4, 2025

Related U.S. Application Data
(60) Provisional application No. 63/503,540, filed on May 22, 2023.

(51) Int. Cl. C07K 16/24; A61K 31/704; A61K 31/7068; A61K 47/68; A61K 49/00; A61K 51/10; C07K 17/08

(52) U.S. Cl. CPC C07K 16/24; A61K 31/704; A61K 31/7068; A61K 47/6845; A61K 49/0058; A61K 51/1021; C07K 17/08; C07K 2317/24; C07K 2317/31; C07K 2317/35

(57) ABSTRACT

This disclosure provides novel neutralizing and potent antibodies targeting S100 proteins, related nucleic acids, related cells, related kits, related compositions, and related methods or uses.

### CROSS-REFERENCE TO RELATED APPLICATIONS

This application claims priority under 35 U.S.C. § 119 (e) to U.S. Provisional Patent Application No. 63/503,540, filed May 22, 2023. The foregoing application is incorporated by reference herein in its entirety.

### FIELD OF THE INVENTION

The present invention relates to antibodies targeting S100 proteins and methods of use thereof.

### BACKGROUND OF THE INVENTION

S100 proteins are a small family of calcium-binding proteins that have cell- and tissue-specific expression. They bind a diverse array of protein targets, have both intracellular and extracellular functions, and regulate multiple cellular processes. The S100A8/A9 homo- and heterodimers are associated with inflammatory and autoimmune diseases, and are elevated in plasma and the tumor microenvironment in solid tumors. Anemia is seen in 90% of myelodysplastic syndromes (MDS) and acute myeloid leukemia (AML) patients and is a major cause of morbidity. Inflammation due to S100A8/9 proteins is the cause of myelodysplastic syndromes' cytopenias. S100A8/9 are involved in propagating the inflammatory response by stimulating leukocyte recruitment and inducing cytokine secretion.

Small molecule inhibitors such as quinoline-3-carboxamides have been explored to treat human autoimmune/inflammatory diseases, particularly multiple sclerosis, before it was reported that its analogs target S100A9. Paquinimod, an analog of this family, inhibits binding of S100A9 to TLR4/MD2 or human RAGE. However, it has no or little effect on binding of S100A8/A9 to these receptors. It was reported that Quinoline-3-carboxamides are not specific inhibitors of S100A9. An open-label clinical trial with paquinimod did not produce expected outcomes (Hesselstrand, R., et al. Arthritis Res Ther 23, 204 (2021)).

Therefore, there is a pressing need for improved inhibitors of S100 proteins for treatment of diseases or disorders, such as inflammatory and autoimmune diseases, leukemia, or myelodysplastic syndromes (MDS).

### SUMMARY OF THE INVENTION

In one aspect, this disclosure provides an isolated antibody or antigen-binding fragment thereof that binds specifically to a S100A8/S100A8 homodimer, a S100A9/S100A9 homodimer, or a S100A8/S100A9 hetero-oligomer. In some embodiments, the isolated antibody or antigen-binding fragment thereof binds specifically to a S100A8/S100A9 hetero-oligomer. In some embodiments, the isolated antibody or antigen-binding fragment thereof binds specifically to a S100A8/S100A9 heterodimer.

In some embodiments, the antibody or antigen-binding fragment thereof comprises: (a) three heavy chain complementarity determining regions (HCDRs) (HCDR1, HCDR2, and HCDR3) contained within a heavy chain variable region (HCVR) having the amino acid sequence of SEQ ID NO: 1; and three light chain CDRs (LCDR1, LCDR2, and LCDR3) contained within a light chain variable region (LCVR) having the amino acid sequence of SEQ ID NO: 2; (b) HCDR1, HCDR2, and HCDR3 of a HCVR having the amino acid sequence of SEQ ID NO: 11; and LCDR1, LCDR2, and LCDR3 of a LCVR having the amino acid sequence of SEQ ID NO: 12; (c) HCDR1, HCDR2, and HCDR3 of a HCVR having the amino acid sequence of SEQ ID NO: 21; and LCDR1, LCDR2, and LCDR3 of a LCVR having the amino acid sequence of SEQ ID NO: 22; (d) HCDR1, HCDR2, and HCDR3 of a HCVR having the amino acid sequence of SEQ ID NO: 31; and LCDR1, LCDR2, and LCDR3 of a LCVR having the amino acid sequence of SEQ ID NO: 32; (e) HCDR1, HCDR2, and HCDR3 of a HCVR having the amino acid sequence of SEQ ID NO: 41; and LCDR1, LCDR2, and LCDR3 of a LCVR having the amino acid sequence of SEQ ID NO: 42; (f) HCDR1, HCDR2, and HCDR3 of a HCVR having the amino acid sequence of SEQ ID NO: 51; and LCDR1, LCDR2, and LCDR3 of a LCVR having the amino acid sequence of SEQ ID NO: 52; (g) HCDR1, HCDR2, and HCDR3 of a HCVR having the amino acid sequence of SEQ ID NO: 61; and LCDR1, LCDR2, and LCDR3 of a LCVR having the amino acid sequence of SEQ ID NO: 62; or (h) HCDR1, HCDR2, and HCDR3 of a HCVR having the amino acid sequence of SEQ ID NO: 71; and LCDR1, LCDR2, and LCDR3 of a LCVR having the amino acid sequence of SEQ ID NO: 72.

In some embodiments, the antibody or antigen-binding fragment thereof comprises HCDR1, HCDR2, HCDR3, LCDR1, LCDR2, and LCDR3 sequences respectively set forth in a CDR sequence set selected from the group consisting of SEQ ID NOs: 3-8, 13-18, 23-28, 33-38, 43-48, 53-58, 63-68, and 73-78.

In some embodiments, the antibody or antigen-binding fragment thereof comprises: (a) a HCVR comprising an amino acid sequence with at least 75% identity to the amino acid sequence of SEQ ID NO: 1 or comprising the amino acid sequence of SEQ ID NO: 1; and a LCVR comprising an amino acid sequence with at least 75% identity to the amino acid sequence of SEQ ID NO: 2 or comprising the amino acid sequence of SEQ ID NO: 2; (b) a HCVR comprising an amino acid sequence with at least 75% identity to the amino acid sequence of SEQ ID NO: 11 or comprising the amino acid sequence of SEQ ID NO: 11; and a LCVR comprising an amino acid sequence with at least 75% identity to the amino acid sequence of SEQ ID NO: 12 or comprising the amino acid sequence of SEQ ID NO: 12; (c) a HCVR comprising an amino acid sequence with at least 75% identity to the amino acid sequence of SEQ ID NO: 21 or comprising the amino acid sequence of SEQ ID NO: 21; and a LCVR comprising an amino acid sequence with at least 75% identity to the amino acid sequence of SEQ ID NO: 22 or comprising the amino acid sequence of SEQ ID NO: 22; (d) a HCVR comprising an amino acid sequence with at least 75% identity to the amino acid sequence of SEQ ID NO: 31 or comprising the amino acid sequence of SEQ ID NO: 31; and a LCVR comprising an amino acid sequence with at least 75% identity to the amino acid sequence of SEQ ID NO: 32 or comprising the amino acid sequence of SEQ ID NO: 32; (e) a HCVR comprising an amino acid sequence with at least 75% identity to the amino acid sequence of SEQ ID NO: 41 or comprising the amino acid sequence of SEQ ID NO: 41; and a LCVR comprising an amino acid sequence with at least 75% identity to the amino acid sequence of SEQ ID NO: 42 or comprising the amino acid sequence of SEQ ID NO: 42; (f) a HCVR comprising an amino acid sequence with at least 75% identity to the amino acid sequence of SEQ ID NO: 51 or comprising the amino acid sequence of SEQ ID NO: 51; and a LCVR comprising an amino acid sequence with at least 75% identity to the amino acid sequence of SEQ ID NO: 52 or comprising the amino acid sequence of SEQ ID NO: 52; (g) a HCVR comprising an amino acid sequence with at least 75% identity to the amino acid sequence of SEQ ID NO: 61 or comprising the amino acid sequence of SEQ ID NO: 61; and a LCVR comprising an amino acid sequence with at least 75% identity to the amino acid sequence of SEQ ID NO: 62 or comprising the amino acid sequence of SEQ ID NO: 62; or (h) a HCVR comprising an amino acid sequence with at least 75% identity to the amino acid sequence of SEQ ID NO: 71 or comprising the amino acid sequence of SEQ ID NO: 71; and a LCVR comprising an amino acid sequence with at least 75% identity to the amino acid sequence of SEQ ID NO: 72 or comprising the amino acid sequence of SEQ ID NO: 72.

In some embodiments, the antibody or antigen-binding fragment thereof comprises a HCVR and a LCVR that comprise a HCVR and LCVR amino acid sequence pair of SEQ ID NOs: 1-2, 11-12, 21-22, 31-32, 41-42, 51-52, 61-62, or 71-72.

In some embodiments, the antibody or antigen-binding fragment thereof comprises a heavy chain (HC) and a light chain (LC) that comprise a HC and LC amino acid sequence pair of SEQ ID NOs: 9-10, 19-20, 29-30, 39-40, 49-50, 59-60, 69-70, or 79-80.

In some embodiments, the antibody or antigen-binding fragment thereof is a 6C8 antibody, a 8H6 antibody, or a 14G9 antibody. In some embodiments, the antibody or antigen-binding fragment thereof is a humanized 8H6 antibody, such as ROIS-1, ROIS-2, ROIS-3, ROIS-4, and ROIS-5.

In some embodiments, the S100A8/S100A9 hetero-oligomer comprises a S100A8/S100A9 heterodimer. In some embodiments, the S100A8/S100A9 hetero-oligomer is a S100A8/S100A9 heterodimer.

In some embodiments, the S100A8 protein is a human S100A8 protein. In some embodiments, the S100A9 protein is a human S100A9 protein.

In some embodiments, the antibody is a monoclonal or polyclonal antibody. In some embodiments, the antibody is a multivalent antibody. In some embodiments, the multivalent antibody is a bivalent or bispecific antibody.

In some embodiments, the antibody is a mouse antibody, a goat antibody, a rabbit antibody, or a human antibody. In some embodiments, the antibody is a chimeric antibody, a humanized antibody, or a humanized monoclonal antibody. In some embodiments, the antibody or antigen-binding fragment thereof comprises a single-chain antibody, Fab or Fab2 fragment. In some embodiments, the antibody or antigen-binding fragment thereof comprises a variant Fc constant region.

In some embodiments, the antibody or antigen-binding fragment thereof is detectably labeled or conjugated to a toxin, a therapeutic agent, a polymer, a receptor, an enzyme, or a receptor ligand, preferably wherein the polymer is polyethylene glycol (PEG).

In another aspect, this disclosure also provides (a) a nucleic acid molecule encoding a polypeptide chain of the antibody or antigen-binding fragment thereof described herein, (b) a vector comprising the nucleic acid molecule described herein, (c) a host cell comprising the nucleic acid molecule or the vector, as described herein.

In yet another aspect, this disclosure further provides a method of preparing an antibody or antigen-binding fragment thereof. In some embodiments, the method comprises: obtaining the host cell described herein; culturing the host cell in a medium under conditions permitting expression of a polypeptide encoded by the nucleic acid molecule or the vector and assembling of an antibody or fragment thereof, and isolating the antibody or antigen-binding fragment thereof from the host cell or the medium.

In another aspect, this disclosure provides a pharmaceutical composition comprising the antibody or antigen-binding fragment thereof, the nucleic acid molecule, or the vector, as described herein; and optionally a pharmaceutically acceptable carrier or excipient. In some embodiments, the pharmaceutical composition is formulated for injection.

Also within the scope of this disclosure is a kit comprising the antibody or antigen-binding fragment thereof, the nucleic acid molecule, the vector, or the pharmaceutical composition, as described herein.

In another aspect, this disclosure also provides a method of treating a disease or disorder in a subject in need thereof. In some embodiments, the method comprises administering to the subject the antibody or antigen-binding fragment thereof, the nucleic acid molecule, the vector, or the pharmaceutical composition, as described herein.

In some embodiments, the disease or disorder comprises leukemia, myelodysplastic syndromes (MDS), or an inflammatory and autoimmune disease.

In some embodiments, the leukemia is acute lymphoblastic leukemia (ALL), acute myeloid leukemia (AML), chronic lymphocytic leukemia (CLL), chronic myeloid leukemia (CML), or chronic myelomonocytic leukemia (CMML).

In some embodiments, the inflammatory and autoimmune disease is non-neoplastic inflammation.

In some embodiments, the method further comprises administering to the subject an additional therapeutic agent or therapy. In some embodiments, the additional therapeutic agent or therapy comprises at least one of daunorubicin, doxorubicin, and cytarabine.

In some embodiments, the antibody or antigen-binding fragment thereof is administered before or after the additional therapeutic agent or therapy. In some embodiments, the antibody or antigen-binding fragment thereof is administered concurrently with the additional therapeutic agent or therapy.

In some embodiments, the antibody or antigen-binding fragment thereof or the additional therapeutic agent or therapy is administered to the subject intravenously, subcutaneously, or intraperitoneally.

In another aspect, this disclosure provides use of an antibody or antigen-binding fragment thereof for the manufacture of a medicament for the method described above.

In another aspect, this disclosure provides an antibody or antigen-binding fragment thereof for use in the method described above.

In another aspect, this disclosure further provides a method of stimulating cell differentiation or cell growth of a cell in a subject in need thereof. In some embodiments, the method comprises administering to the subject the antibody or antigen-binding fragment thereof, the nucleic acid molecule, the vector, or the pharmaceutical composition, as described herein.

In yet another aspect, this disclosure additionally provides a method of stimulating cell differentiation or cell growth of a cell ex vivo. In some embodiments, the method comprises contacting the cell with the antibody or antigen-binding fragment thereof, the nucleic acid molecule, the vector, or the pharmaceutical composition, as described herein.

In some embodiments, the cell comprises an acute myeloid leukemia cell or a red blood cell.

In some embodiments, the subject has leukemia or a myelodysplastic syndrome (MDS). In some embodiments, the leukemia is acute lymphoblastic leukemia (ALL), acute myeloid leukemia (AML), chronic lymphocytic leukemia (CLL), chronic myeloid leukemia (CML), or chronic myelomonocytic leukemia (CMML).

In another aspect, this disclosure provides a method for detecting the presence of a S100 protein in a sample. In some embodiments, the method comprises: (i) contacting the sample with the antibody or antigen-binding fragment thereof described above; and (ii) determining binding of the antibody or antigen-binding fragment to the alarmin protein, wherein binding of the antibody or antigen-binding fragment thereof to the S100 protein is indicative of the presence of the alarmin protein in the sample.

In some embodiments, the S100 protein comprise a S100A8/S100A8 homodimer, a S100A9/S100A9 homodimer, or a S100A8/S100A9 hetero-oligomer (such as a S100A8/S100A9 heterodimer).

In some embodiments, the antibody or antigen-binding fragment thereof is conjugated to a label. In some embodiments, the label is selected from a fluorescent label, a chemiluminescent label, a radiolabel, and an enzyme.

In some embodiments, the method comprises contacting a secondary antibody with the antibody or antigen-binding fragment thereof. In some embodiments, the step of determining comprises performing a competitive binding assay or ELISA.

In some embodiments, the sample comprises a blood sample. In some embodiments, the method comprises binding the sample to a solid support. In some embodiments, the solid support is selected from microparticles, microbeads, magnetic beads, and an affinity purification column.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows affinity of antisera from three rabbits for S100A8/A9, S100A8, and S100A9 (Kactus Biosystems or Sino Biological). Rabbit #8944 showed the highest affinity for S100A8/S100A9 and was chosen for subsequent processes. The antiserum from #8944-II was used extensively to define the activity of monoclonal antibodies later.

FIG. 2 shows specificity of final three rabbit monoclonal antibodies for S100A8, S100A9 and S100A8/A9. S100A8/A9 proteins from two additional companies were included. In this experiment, S100A8, S100A9 or S100A8/A9 heterodimer was immobilized on ELISA plate and probed by rabbit monoclonal antibodies, followed by rabbit-horseradish peroxidase conjugate antibody before standard color development.

FIGS. 3A, 3B, and 3C show inhibitory effects of monoclonal antibodies on binding of S100A8 (FIG. 3A), S100A9 (FIG. 3B), and S100A8/S100A9 (FIG. 3C) to human RAGE-Fc in TBS+Ca²⁺ buffer by ELISA. Rabbit IgG serves as a negative control. Human RAGE-Fc is immobilized and incubated with S100A proteins in the presence or absence of the antibody. The bound S100A proteins were detected with mouse anti-His-tag antibody followed by mouse IgG horseradish peroxidase conjugate antibody since all of these proteins are His-tagged.

FIG. 4 shows dose-dependent suppression of S100A8/A9-Kactus (1 μg/ml) binding to RAGE-Fc by antibody in TBS+Ca²⁺ buffer. Binding of S100A proteins to the immobilized human RAGE-Fc in the presence or absence of rabbit monoclonal antibodies was detected with mouse anti-His-tag antibody.

FIG. 5 shows retention of specificity of rabbit/mouse chimera antibodies for various S100A proteins. Both chimera antibodies have an equally high affinity for S100A8/A9 proteins. S100A proteins were immobilized on an ELISA plate and probed with the chimera antibodies followed by mouse IgG horseradish peroxidase conjugate antibody. Both chimera antibodies have an equally high affinity for all S100A8/A9 proteins obtained. Unlike the other sources of S100A8/A9 proteins, which are recombinant, S100A8/A9-Arotec was purified from human neutrophils.

FIGS. 6A and 6B show marked suppression of S100A-8-Sino binding by 8H6-mIgG2a, S1000-9-Sino binding by 6C8-mIgG2a (FIG. 6A) or S100A8/A9-Sino binding by both (FIG. 6B) to receptors in TBS+Ca²⁺+Zn²⁺ buffer. S100A8-Sino, S100A9-Sino or S100A8/A9-Sino were at 1 μg/ml, while both 8H6-mIgG2a or 6C8-IgG2a were at 10 μg/ml. Mouse IgG serves as a negative control. As described previously, receptor proteins were immobilized on ELISA plates and incubated with S100A proteins in the presence or absence of chimera antibodies. The plate-bound S100A proteins were detected by antiserum 9844-II followed by rabbit IgG horseradish peroxidase conjugate antibody.

FIG. 7 shows dose-dependent suppression of S100A8/A9-Sino (1 μg/ml) binding to CD69 by antibody in TBS+Ca²⁺+Zn²⁺ buffer. CD69 was immobilized on an ELISA plate and incubated with S100A8/A9-Sino in the presence or absence of chimera antibodies. The plate-bound S100A proteins were detected by antiserum 9844-II followed by rabbit IgG horseradish peroxidase conjugate antibody.

FIG. 8 shows binding of S100A8/A9 from different sources (1 μg/ml) to RAGE-Fc in the presence of antibodies at 10 and 2.5 μg/ml in TBS+Ca²⁺ buffer. RAGE-Fc was immobilized on an ELISA plate and incubated with S100A8/A9 in the presence or absence of chimera antibodies. The plate-bound S100A proteins were detected by antiserum 9844-II followed by rabbit IgG horseradish peroxidase conjugate antibody.

FIG. 9 shows the difference between 6C8-mIgG2a and 8H6-mIgG2a for their inhibition on S100A8/A9-Arotec binding (1 μg/ml) to human RAGE-Fc in TBS+Ca²⁺ buffer. RAGE-Fc was immobilized on an ELISA plate and incubated with S100A8/A9-Arotec in the absence or presence of chimera antibodies at three different concentrations. The plate-bound S100A8/A9 was detected by antiserum 9844-II followed by rabbit IgG horseradish peroxidase conjugate antibody. 8H6-mIg2a was two-fold more potent than 6C8-mIgG2a to neutralize S100A8/A9-Arotec.

FIGS. 10A and 10B show the impacts of S100A8/A9 protein, neutralizing antibody 6C8-mIgG2a, or in combination on CD3/CD28 antibody-induction of regulatory T cells (Treg). PBMCs were activated by CD3/CD28 antibodies in the presence or absence of human S100A8/A9 recombinant protein (2.5 ug/ml) and/or neutralizing antibody 6C8-mIgG2a (5 μg/ml) for 5 days. The presence of Treg cells (CD25+Foxp3+) was quantitated by routine flow cytometry. While FIG. 10A shows one representative experiment, FIG. 10B displays the mean±SEM of five independent experiments. These results indicate that 6C8-mIgG2a can reverse the S100A8/A9-enhanced Treg cell induction when T cells are activated.

FIG. 11 shows the inhibition of S100A8/A9 binding to CD69 by humanized 8H6 antibodies. CD69 was immobilized on an ELISA plate and incubated with S100A8/A9-Sino (1 ug/ml) in the presence or absence of humanized 8H6 antibodies with 8H6-HC (human chimera) as a positive control and human serum IgG as a negative control. Three different antibody concentrations (10, 2.5, and 1.25 ug/ml) were chosen for more accurate comparison between antibodies. The plate-bound S100A8/A9 protein was detected with 6C8-mIgG2a followed by HRP-conjugated anti-Mouse IgG. The data are representative of three independent experiments and indicate that 8H6 has been successfully humanized, with ROIS-3 having a much higher potential than the human chimera to neutralize S100A8/A9.

### DETAILED DESCRIPTION OF THE INVENTION

This disclosure provides novel antibodies targeting S100 proteins, such as a S100A8/S100A8 homodimer, a S100A9/S100A9 homodimer, or a S100A8/S100A9 hetero-oligomer (such as a S100A8/S100A9 heterodimer). The disclosed antibodies unexpectedly exhibit high binding affinity and high specificity against the S100A8/S100A9 hetero-oligomer, and can be used in various immunotherapies for treating S100 proteins-related diseases or disorders.

### Antibodies Targeting S100 Proteins

### Antibodies

In one aspect, this disclosure provides an isolated antibody or antigen-binding fragment thereof that binds specifically to a S100A8/S100A8 homodimer, a S100A9/S100A9 homodimer, or a S100A8/S100A9 hetero-oligomer (such as a S100A8/S100A9 heterodimer).

S100 proteins are involved in both intracellular and extracellular processes, including cell apoptosis, migration, protein phosphorylation, calcium balance, differentiation, proliferation, and inflammation (Zeeshan Sattar, et al. Pulmonary Medicine, vol. 2021, Article ID 5488591 (2021)). The term S100 was first used to describe proteins that were soluble in 100% saturated ammonium sulfate in 1965. There are now 25 such S100 proteins/complexes described, which include 16 S100A proteins (S100A1-S100A16) as well as others (such as S100B, S100G, S100P, and S100Z). The S100 protein family is exclusively expressed in vertebrates. It consists of small (10-14 kDa), acidic, and calcium-binding proteins (CaBPs) with two distinct EF-hand motifs (helix-loop-helix). This subfamily of CaBPs exists mainly as homodimers but can also exist as monomers (only S100G is stable in this configuration), heterodimers (S100A1/S100B and S100A8/S100A9), or multimers intra- and extracellularly. Their expression is tissue and cell-type specific. Along with Ca²⁻, S100 proteins also bind many other transition metal ions (e.g., Fe²⁺, Cu²⁺, Mn²⁺, Zn²⁺, and Ni²⁺) that result in a conformational change allowing interaction with target proteins. S100 proteins act as DAMP molecules and can work as stimulatory ligands for both immune and nonimmune cells, such as endothelial cells. They do so by binding pattern recognition receptors (PRRs) such as toll-like receptor 4 (TLR4) as well as non-PRR DAMP receptors such as advanced glycation end products (RAGE). By binding to these receptors, S100 proteins trigger downstream nuclear factor-κB (NF-κB), which results in upregulation of the pro-inflammatory gene expression.

Alarmins S100A8 and S100A9 are endogenous molecules released in response to environmental triggers and cellular damage. They are constitutively expressed in immune cells such as monocytes and neutrophils, and their expression is upregulated under inflammatory conditions. S100A8 and S100A9 can form non-covalently associated oligomers, such as monovalent S100A8 or S100A9 homodimers and S100A8/A9 heterodimers (MRP8/14, calprotectin), as well as even higher oligomeric forms (Hunter and Chazin, J Biol Chem (1998) 273 (20): 12427-35, Vogl et al., J Am Soc Mass Spectrom (1999) 10:1124-1130). In this context, distinct hydrophobic amino acids have been identified as directly involved in S100A8/S100A9 dimer formation (Leukert et al., Biol Chem (2005), 386:429-434). S100A8 and S100A9 have also been found to oligomerize to (S100A8/S100A9) 2 heterotetramers. Tetramer formation is strictly dependent on the presence of calcium, and in the absence of calcium, heterodimers are the predominant forms of S100A8 and S100A9 hetero-oligomers. The dimer form is known to bind four Ca²⁺-ions, while the (S100A8/S100A9) 2 heterotetramer binds eight Ca²⁺-ions. S100A8 and S100A9 represent the major calcium-binding proteins in phagocytes, and both proteins regulate migration of these cells via modulation of tubulin polymerization. In biological samples, S100A8 and S100A9 generally exist as heterodimers and tetramers. S100A8/S100A9 functions as an endogenous TLR4 ligand due to their specific and high expression at sites of inflammation. Accordingly, S100A8/S100A9 heterodimers can be considered as early amplifiers of inflammation, inducing pro-inflammatory responses in endothelial cells and phagocytes.

In one aspect, this disclosure provides an isolated antibody or antigen-binding fragment thereof that binds specifically to a S100A8/S100A9 hetero-oligomer (such as a S100A8/S100A9 hetero-tetramer, a S100A8/S100A9 heterodimer). In some embodiments, the isolated antibody or antigen-binding fragment thereof binds specifically to a S100A8/S100A9 heterodimer.

In some embodiments, the antibody or antigen-binding fragment thereof comprises: three heavy chain complementarity determining regions (HCDRs) (HCDR1, HCDR2, and HCDR3) contained within a heavy chain variable region (HCVR) having the amino acid sequence of SEQ ID NO: 1; and three light chain CDRs (LCDR1, LCDR2, and LCDR3) contained within a light chain variable region (LCVR) having the amino acid sequence of SEQ ID NO: 2.

In some embodiments, the antibody or antigen-binding fragment thereof comprises: HCDR1, HCDR2, and HCDR3 of a HCVR having the amino acid sequence of SEQ ID NO: 11; and LCDR1, LCDR2, and LCDR3 of a LCVR having the amino acid sequence of SEQ ID NO: 12.

In some embodiments, the antibody or antigen-binding fragment thereof comprises: HCDR1, HCDR2, and HCDR3 of a HCVR having the amino acid sequence of SEQ ID NO: 21; and LCDR1, LCDR2, and LCDR3 of a LCVR having the amino acid sequence of SEQ ID NO: 22.

In some embodiments, the antibody or antigen-binding fragment thereof comprises: HCDR1, HCDR2, and HCDR3 of a HCVR having the amino acid sequence of SEQ ID NO: 31; and LCDR1, LCDR2, and LCDR3 of a LCVR having the amino acid sequence of SEQ ID NO: 32.

In some embodiments, the antibody or antigen-binding fragment thereof comprises: HCDR1, HCDR2, and HCDR3 of a HCVR having the amino acid sequence of SEQ ID NO: 41; and LCDR1, LCDR2, and LCDR3 of a LCVR having the amino acid sequence of SEQ ID NO: 42.

In some embodiments, the antibody or antigen-binding fragment thereof comprises: HCDR1, HCDR2, and HCDR3 of a HCVR having the amino acid sequence of SEQ ID NO: 51; and LCDR1, LCDR2, and LCDR3 of a LCVR having the amino acid sequence of SEQ ID NO: 52.

In some embodiments, the antibody or antigen-binding fragment thereof comprises: HCDR1, HCDR2, and HCDR3 of a HCVR having the amino acid sequence of SEQ ID NO: 61; and LCDR1, LCDR2, and LCDR3 of a LCVR having the amino acid sequence of SEQ ID NO: 62.

In some embodiments, the antibody or antigen-binding fragment thereof comprises: HCDR1, HCDR2, and HCDR3 of a HCVR having the amino acid sequence of SEQ ID NO: 71; and LCDR1, LCDR2, and LCDR3 of a LCVR having the amino acid sequence of SEQ ID NO: 72

In some embodiments, the antibody or antigen-binding fragment thereof comprises HCDR1, HCDR2, HCDR3, LCDR1, LCDR2, and LCDR3 sequences respectively set forth in a CDR sequence set of SEQ ID NOs: 3-8.

In some embodiments, the antibody or antigen-binding fragment thereof comprises HCDR1, HCDR2, HCDR3, LCDR1, LCDR2, and LCDR3 sequences respectively set forth in a CDR sequence set of SEQ ID NOs: 13-18.

In some embodiments, the antibody or antigen-binding fragment thereof comprises HCDR1, HCDR2, HCDR3, LCDR1, LCDR2, and LCDR3 sequences respectively set forth in a CDR sequence set of SEQ ID NOs: 23-28.

In some embodiments, the antibody or antigen-binding fragment thereof comprises HCDR1, HCDR2, HCDR3, LCDR1, LCDR2, and LCDR3 sequences respectively set forth in a CDR sequence set of SEQ ID NOs: 33-38.

In some embodiments, the antibody or antigen-binding fragment thereof comprises HCDR1, HCDR2, HCDR3, LCDR1, LCDR2, and LCDR3 sequences respectively set forth in a CDR sequence set of SEQ ID NOs: 43-48.

In some embodiments, the antibody or antigen-binding fragment thereof comprises HCDR1, HCDR2, HCDR3, LCDR1, LCDR2, and LCDR3 sequences respectively set forth in a CDR sequence set of SEQ ID NOs: 53-58.

In some embodiments, the antibody or antigen-binding fragment thereof comprises HCDR1, HCDR2, HCDR3, LCDR1, LCDR2, and LCDR3 sequences respectively set forth in a CDR sequence set of SEQ ID NOs: 63-68.

In some embodiments, the antibody or antigen-binding fragment thereof comprises HCDR1, HCDR2, HCDR3, LCDR1, LCDR2, and LCDR3 sequences respectively set forth in a CDR sequence set of SEQ ID NOs: 73-78.

In some embodiments, the antibody or antigen-binding fragment thereof comprises: a HCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 1 or comprising the amino acid sequence of SEQ ID NO: 1; and a LCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 2 or comprising the amino acid sequence of SEQ ID NO: 2.

In some embodiments, the antibody or antigen-binding fragment thereof comprises: a HCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 11 or comprising the amino acid sequence of SEQ ID NO: 11; and a LCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 12 or comprising the amino acid sequence of SEQ ID NO: 12.

In some embodiments, the antibody or antigen-binding fragment thereof comprises: a HCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 21 or comprising the amino acid sequence of SEQ ID NO: 21; and a LCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 22 or comprising the amino acid sequence of SEQ ID NO: 22.

In some embodiments, the antibody or antigen-binding fragment thereof comprises: a HCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 31 or comprising the amino acid sequence of SEQ ID NO: 31; and a LCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 32 or comprising the amino acid sequence of SEQ ID NO: 32.

In some embodiments, the antibody or antigen-binding fragment thereof comprises: a HCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 41 or comprising the amino acid sequence of SEQ ID NO: 41; and a LCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 42 or comprising the amino acid sequence of SEQ ID NO: 42.

In some embodiments, the antibody or antigen-binding fragment thereof comprises: a HCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 51 or comprising the amino acid sequence of SEQ ID NO: 51; and a LCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 52 or comprising the amino acid sequence of SEQ ID NO: 52.

In some embodiments, the antibody or antigen-binding fragment thereof comprises: a HCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 61 or comprising the amino acid sequence of SEQ ID NO: 61; and a LCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 62 or comprising the amino acid sequence of SEQ ID NO: 62.

In some embodiments, the antibody or antigen-binding fragment thereof comprises: a HCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 71 or comprising the amino acid sequence of SEQ ID NO: 71; and a LCVR comprising an amino acid sequence with at least 75% (e.g., 75%, 78%, 80%, 82%, 84%, 85%, 86%, 88%, 90%, 92%, 94%, 95%, 96%, 97%, 98%, 99%) identity to the amino acid sequence of SEQ ID NO: 72 or comprising the amino acid sequence of SEQ ID NO: 72.

In some embodiments, the antibody or antigen-binding fragment thereof comprises a HCVR and a LCVR that comprise a HCVR and LCVR amino acid sequence pair of SEQ ID NOs: 1-2.

In some embodiments, the antibody or antigen-binding fragment thereof comprises a HCVR and a LCVR that comprise a HCVR and LCVR amino acid sequence pair of SEQ ID NOs: 11-12.

In some embodiments, the antibody or antigen-binding fragment thereof comprises a HCVR and a LCVR that comprise a HCVR and LCVR amino acid sequence pair of SEQ ID NOs: 21-22.

In some embodiments, the antibody or antigen-binding fragment thereof comprises a HCVR and a LCVR that comprise a HCVR and LCVR amino acid sequence pair of SEQ ID NOs: 31-32.

In some embodiments, the antibody or antigen-binding fragment thereof comprises a HCVR and a LCVR that comprise a HCVR and LCVR amino acid sequence pair of SEQ ID NOs: 41-42.

In some embodiments, the antibody or antigen-binding fragment thereof comprises a HCVR and a LCVR that comprise a HCVR and LCVR amino acid sequence pair of SEQ ID NOs: 51-52.

In some embodiments, the antibody or antigen-binding fragment thereof comprises a HCVR and a LCVR that comprise a HCVR and LCVR amino acid sequence pair of SEQ ID NOs: 61-62.

In some embodiments, the antibody or antigen-binding fragment thereof comprises a HCVR and a LCVR that comprise a HCVR and LCVR amino acid sequence pair of SEQ ID NOs: 71-72.

In some embodiments, the antibody or antigen-binding fragment thereof comprises a heavy chain (HC) and a light chain (LC) that comprise a HC and LC amino acid sequence pair of SEQ ID NOs: 9-10.

In some embodiments, the antibody or antigen-binding fragment thereof comprises a HC and a LC that comprise a HC and LC amino acid sequence pair of SEQ ID NOs: 19-20.

In some embodiments, the antibody or antigen-binding fragment thereof comprises a HC and a LC that comprise a HC and LC amino acid sequence pair of SEQ ID NOs: 29-30.

In some embodiments, the antibody or antigen-binding fragment thereof comprises a HC and a LC that comprise a HC and LC amino acid sequence pair of SEQ ID NOs: 39-40.

In some embodiments, the antibody or antigen-binding fragment thereof comprises a HC and a LC that comprise a HC and LC amino acid sequence pair of SEQ ID NOs: 49-50.

In some embodiments, the antibody or antigen-binding fragment thereof comprises a HC and a LC that comprise a HC and LC amino acid sequence pair of SEQ ID NOs: 59-60.

In some embodiments, the antibody or antigen-binding fragment thereof comprises a HC and a LC that comprise a HC and LC amino acid sequence pair of SEQ ID NOs: 69-70.

In some embodiments, the antibody or antigen-binding fragment thereof comprises a HC and a LC that comprise a HC and LC amino acid sequence pair of SEQ ID NOs: 79-80.

In some embodiments, the antibody or antigen-binding fragment thereof is a 6C8 antibody, a 8H6 antibody, or a 14G9 antibody. The 6C8 antibody binds the S100A9/S100A9 homodimer or the S100A8/S100A9 heterodimer; 8H6 binds S100A8/S100A8 homodimer or the S100A8/S100A9 heterodimer; 14G9 binds the S100A8/S100A9 heterodimer only.

In some embodiments, the S100A8 protein is a human S100A8 protein. In some embodiments, the S100A9 protein is a human S100A9 protein.

In some embodiments, the antibody is a monoclonal or polyclonal antibody. In some embodiments, the antibody is a multivalent antibody. In some embodiments, the multivalent antibody is a bivalent or bispecific antibody.

In some embodiments, the antibody is a mouse antibody, a goat antibody, a rabbit antibody, or a human antibody. In some embodiments, the antibody or the antigen-binding fragment thereof comprises a variant Fc constant region.

In some embodiments, the antibody can be a chimeric antibody, a humanized antibody, or a humanized monoclonal antibody. In some embodiments, the antibody can be a single-chain antibody, Fab or Fab2 fragment.

TABLE 1
Amino acid sequences of the representative antibodies

| | | OTHER | SEQ ID |
|---|---|---|---|
| ANTIBODY | SEQUENCE | INFORMATION | NO |
| 6C8 | QEQLVESGGGLVTLGGSLKLSCKASGIDFSIYGISWV | HCVR | 1 |
| | RQAPGKGLEWIAYIYPDYSTTDYASWVNGRFTISFDN | | |
| | AQNTVFLEMTSLTAADTATYFCARRYSGYYGLWGP | | |
| | GTLVTVSS | | |
| | ALVMTQTPSSVSAPVGGTVTIKCQASQSISSYLAWYQ | LCVR | 2 |
| | QKPGQRPKLLIYGASSLESGVPSRFKGSGSGTEYTLTI | | |
| | SDLECDDAATYYCQGAHAGSSADAFGGGTEVVVKG | | |
| | DP | | |
| | IYGIS | HCDR1 | 3 |
| | YIYPDYSTTDYASWVNG | HCDR2 | 4 |
| | RYSGYYGL | HCDR3 | 5 |
| | QASQSISSYLA | LCDR1 | 6 |
| | GASSLES | LCDR2 | 7 |
| | QGAHAGSSADA | LCDR3 | 8 |
| | METGLRWLLLVAVLKGVQCQEQLVESGGGLVTLGG | HC | 9 |
| | SLKLSCKASGIDFSIYGISWVRQAPGKGLEWIAYIYPD | | |
| | YSTTDYASWVNGRFTISFDNAQNTVFLEMTSLTAAD | | |
| | TATYFCARRYSGYYGLWGPGTLVTVSSGQPKAPSVF | | |
| | PLAPCCGDTPSSTVTLGCLVKGYLPEPVTVTWNSGTL | | |
| | TNGVRTFPSVRQSSGLYSLSSVVSVTSSSQPVTCNVA | | |
| | HPATNTKVDKTVAPSTCSKPMCPPPELPGGPSVFIFPP | | |
| | KPKDTLMISRTPEVTCVVVDVSQDDPEVQFTWYINN | | |
| | EQVRTARPPLREQQFNSTIRVVSTLPIAHQDWLRGKE | | |
| | FKCKVHNKALPAPIEKTISKARGQPLEPKVYTMGPPR | | |
| | EELSSRSVSLTCMINGFYPSDISVEWEKNGKAEDNYK | | |
| | TTPTVLDSDGSYFLYSKLSVPTSEWQRGDVFTCSVM | | |
| | HEALHNHYTQKSISRSPGK | | |
| | MDTRAPTQLLGLLLLWLPGARCALVMTQTPSSVSAP | LC | 10 |
| | VGGTVTIKCQASQSISSYLAWYQQKPGQRPKLLIYGA | | |
| | SSLESGVPSRFKGSGSGTEYTLTISDLECDDAATYYC | | |
| | QGAHAGSSADAFGGGTEVVVKGDPVAPTVLIFPPAA | | |
| | DQVATGTVTIVCVANKYFPDVTVTWEVDGTTQTTGI | | |
| | ENSKTPQNSADCTYNLSSTLTLTSTQYNSHKEYTCKV | | |
| | TQGTTSVVQSFNRGDC | | |
| 8H6 | CQSLEESGGDLVKPEGSLTLTCKASGFSFSSGCWVC | HCVR | 11 |
| | WVRQAPGKGLEWIACIYTGDGDTYYANWARGRFTV | | |
| | SRTSSTTVTLQLTSLTAADTATYFCASPQPGDITYLGF | | |
| | WGPGTLVTVSS | | |
| | DIVMTQTPASVSEPVGGTVTIKCQASQNINSYLSWYQ | LCVR | 12 |
| | QRPGQPPKLLISLASDLASGVPSRFKGSESGTDYTLTI | | |
| | SDLECADAATYYCQGYFADSGSSGRSWAFGGGTEV | | |
| | VVQGDP | | |
| | SGCWVC | HCDR1 | 13 |
| | CIYTGDGDTYYANWARG | HCDR2 | 14 |
| | PQPGDITYLGF | HCDR3 | 15 |
| | QASQNINSYLS | LCDR1 | 16 |
| | LASDLAS | LCDR2 | 17 |
| | QGYFADSGSSGRSWA | LCDR3 | 18 |
| | METGLRWLLLVAVLKGVQCQSLEESGGDLVKPEGSL | HC | 19 |
| | TLTCKASGFSFSSGCWVCWVRQAPGKGLEWIACIYT | | |
| | GDGDTYYANWARGRFTVSRTSSTTVTLQLTSLTAAD | | |
| | TATYFCASPQPGDITYLGFWGPGTLVTVSSGQPKAPS | | |
| | VFPLAPCCGDTPSSTVTLGCLVKGYLPEPVTVTWNSG | | |
| | TLTNGVRTFPSVRQSSGLYSLSSVVSVTSSSQPVTCN | | |
| | VAHPATNTKVDKTVAPSTCSKPMCPPPELPGGPSVFI | | |
| | FPPKPKDTLMISRTPEVTCVVVDVSQDDPEVQFTWYI | | |
| | NNEQVRTARPPLREQQFNSTIRVVSTLPIAHQDWLRG | | |
| | KEFKCKVHNKALPAPIEKTISKARGQPLEPKVYTMGP | | |
| | PREELSSRSVSLTCMINGFYPSDISVEWEKNGKAEDN | | |
| | YKTTPTVLDSDGSYFLYSKLSVPTSEWQRGDVFTCSV | | |
| | MHEALHNHYTQKSISRSPGK | | |
| | MDTRAPTQLLGLLLLWLPGARCADIVMTQTPASVSE | LC | 20 |
| | PVGGTVTIKCQASQNINSYLSWYQQRPGQPPKLLISL | | |
| | ASDLASGVPSRFKGSESGTDYTLTISDLECADAATYY | | |
| | CQGYFADSGSSGRSWAFGGGTEVVVQGDPVAPTVLI | | |
| | FPPATDQVATGTVTIVCVANKYFPDVTVTWEVDGTT | | |
| | QTTGIENSKTPQNSADCTYNLSSTLTLTSTQYNSHKE | | |
| | YTCKVTQGTTSVVQSFNRGDC | | |
| 14G9 | QEQLVESGGGLVQPEGSLTLTCTASGFSFSSDYYMC | HCVR | 21 |
| | WVRQAPGKGLEWIACIGGGYGGSPYYATWAKGRFTI | | |
| | SKTSSTAVTLQMTSLTAADTATHFCARGVPGSSCFFD | | |
| | LWGPGTLVTVSS | | |
| | DVAMTQTPASVSEPVGGTVTIKCQASQSIGSSLAWY | LCVR | 22 |
| | QQKSGQPPKLLIYDAFDLASGVSSRFKGSGSGTEYTL | | |
| | TISDLECADAATYFCLGGYYDANTFGGGTEVVVKGD | | |
| | P | | |
| | SDYYMC | HCDR1 | 23 |
| | CIGGGYGGSPYYATWAKG | HCDR2 | 24 |
| | GVPGSSCFFDL | HCDR3 | 25 |
| | QASQSIGSSLA | LCDR1 | 26 |
| | DAFDLAS | LCDR2 | 27 |
| | LGGYYDANT | LCDR3 | 28 |
| | METGLRWLLLVAVLKGVQCQEQLVESGGGLVQPEG | HC | 29 |
| | SLTLTCTASGFSFSSDYYMCWVRQAPGKGLEWIACIG | | |
| | GGYGGSPYYATWAKGRFTISKTSSTAVTLQMTSLTA | | |
| | ADTATHFCARGVPGSSCFFDLWGPGTLVTVSSGQPK | | |
| | APSVFPLAPCCGDTPSSTVTLGCLVKGYLPEPVTVTW | | |
| | NSGTLTNGVRTFPSVRQSSGLYSLSSVVSVTSSSQPVT | | |
| | CNVAHPATNTKVDKTVAPSTCSKPMCPPPELPGGPS | | |
| | VFIFPPKPKDTLMISRTPEVTCVVVDVSQDDPEVQFT | | |
| | WYINNEQVRTARPPLREQQFNSTIRVVSTLPIAHQDW | | |
| | LRGKEFKCKVHNKALPAPIEKTISKARGQPLEPKVYT | | |
| | MGPPREELSSRSVSLTCMINGFYPSDISVEWEKNGKA | | |
| | EDNYKTTPTVLDSDGSYFLYSKLSVPTSEWQRGDVF | | |
| | TCSVMHEALHNHYTQKSISRSPGK | | |
| | MDTRAPTQLLGLLLLWLPGASCDVAMTQTPASVSEP | LC | 30 |
| | VGGTVTIKCQASQSIGSSLAWYQQKSGQPPKLLIYDA | | |
| | FDLASGVSSRFKGSGSGTEYTLTISDLECADAATYFC | | |
| | LGGYYDANTFGGGTEVVVKGDPVAPTVLIFPPAADQ | | |
| | VATGTVTIVCVANKYFPDVTVTWEVDGTTQTTGIEN | | |
| | SKTPQNSADCTYNLSSTLTLTSTQYNSHKEYTCKVTQ | | |
| | GTTSVVQSFNRGDC | | |
| ROIS-1 | NVQLVESGGGLIHPGGSLSLSCKASGFSFSSGCWVC | HCVR | 31 |
| | WVRQAPGKGLEWVACIYTGDGDTYYADSVRGRFTIS | | |
| | RTSSKTTVYLQMNSLRAEDTAVYYCASPQPGDITYL | | |
| | GFWGQGTLVTVSS | | |
| | DIHMTQSPSSLSASVGDTVTITCQASQNINSYLSWYQ | LCVR | 32 |
| | QEPGKAPKLLISLASDLASGVPSRFSGSESGTDYTLTI | | |
| | SSLQPEDFATYYCQGYFADSGSSGRSWAFGQGTKVE | | |
| | IKRTV | | |
| | SGCWVC | HCDR1 | 33 |
| | CIYTGDGDTYYADSVRG | HCDR2 | 34 |
| | PQPGDITYLGF | HCDR3 | 35 |
| | QASQNINSYLS | LCDR1 | 36 |
| | LASDLAS | LCDR2 | 37 |
| | QGYFADSGSSGRSWA | LCDR3 | 38 |
| | METGLRWLLLVAVLKGVQNVQLVESGGGLIHPGGSL | HC | 39 |
| | SLSCKASGFSFSSGCWVCWVRQAPGKGLEWVACIYT | | |
| | GDGDTYYADSVRGRFTISRTSSKTTVYLQMNSLRAE | | |
| | DTAVYYCASPQPGDITYLGFWGQGTLVTVSSASTKG | | |
| | PSVFPLAPCSRSTSESTAALGCLVKDYFPEPVTVSWN | | |
| | SGALTSGVHTFPAVLQSSGLYSLSSVVTVPSSSLGTKT | | |
| | YTCNVDHKPSNTKVDKRVESKYGPPCPPCPAPEFLG | | |
| | GPSVFLFPPKPKDTLMISRTPEVTCVVVDVSQEDPEV | | |
| | QFNWYVDGVEVHNAKTKPREEQFNSTYRVVSVLTV | | |
| | LHQDWLNGKEYKCKVSNKGLPSSIEKTISKAKGQPR | | |
| | EPQVYTLPPSQEEMTKNQVSLTCLVKGFYPSDIAVEW | | |
| | ESNGQPENNYKTTPPVLDSDGSFFLYSRLTVDKSRW | | |
| | QEGNVFSCSVMHEALHNHYTQKSLSLSLGK | | |
| | MDTRAPTQLLGLLLLWLPGARCADIHMTQSPSSLSAS | LC | 40 |
| | VGDTVTITCQASQNINSYLSWYQQEPGKAPKLLISLA | | |
| | SDLASGVPSRFSGSESGTDYTLTISSLQPEDFATYYCQ | | |
| | GYFADSGSSGRSWAFGQGTKVEIKRTVAAPSVFIFPP | | |
| | SDEQLKSGTASVVCLLNNFYPREAKVQWKVDNALQ | | |
| | SGNSQESVTEQDSKDSTYSLSSTLTLSKADYEKHKVY | | |
| | ACEVTHQGLSSPVTKSFNRGEC | | |
| ROIS-2 | NVQLVESGGGLIHPGGSLSLSCKASGFSFSSGCWVC | HCVR | 41 |
| | WVRQAPGKGLEWVACIYTGDGDTYYADSVKGRFTI | | |
| | SRTSSKTTVYLQMNSLRAEDTAVYYCASPQPGDITYL | | |
| | GFWGQGTLVTVSS | | |
| | DIHMTQSPSSLSASVGDTVTITCQASQNINSYLSWYQ | LCVR | 42 |
| | QKPGKAPKLLISLASDLASGVPSRFSGSESGTDYTLTI | | |
| | SSLQPEDFATYYCQGYFADSGSSGRSWAFGQGTKVE | | |
| | IKRTV | | |
| | SGCWVC | HCDR1 | 43 |
| | CIYTGDGDTYYADSVKG | HCDR2 | 44 |
| | PQPGDITYLGF | HCDR3 | 45 |
| | QASQNINSYLS | LCDR1 | 46 |
| | LASDLAS | LCDR2 | 47 |
| | QGYFADSGSSGRSWA | LCDR3 | 48 |
| | METGLRWLLLVAVLKGVQNVQLVESGGGLIHPGGSL | HC | 49 |
| | SLSCKASGFSFSSGCWVCWVRQAPGKGLEWVACIYT | | |
| | GDGDTYYADSVKGRFTISRTSSKTTVYLQMNSLRAE | | |
| | DTAVYYCASPQPGDITYLGFWGQGTLVTVSSASTKG | | |
| | PSVFPLAPCSRSTSESTAALGCLVKDYFPEPVTVSWN | | |
| | SGALTSGVHTFPAVLQSSGLYSLSSVVTVPSSSLGTKT | | |
| | YTCNVDHKPSNTKVDKRVESKYGPPCPPCPAPEFLG | | |
| | GPSVFLFPPKPKDTLMISRTPEVTCVVVDVSQEDPEV | | |
| | QFNWYVDGVEVHNAKTKPREEQFNSTYRVVSVLTV | | |
| | LHQDWLNGKEYKCKVSNKGLPSSIEKTISKAKGQPR | | |
| | EPQVYTLPPSQEEMTKNQVSLTCLVKGFYPSDIAVEW | | |
| | ESNGQPENNYKTTPPVLDSDGSFFLYSRLTVDKSRW | | |
| | QEGNVFSCSVMHEALHNHYTQKSLSLSLGK | | |
| | MDTRAPTQLLGLLLLWLPGARCADIHMTQSPSSLSAS | LC | 50 |
| | VGDTVTITCQASQNINSYLSWYQQKPGKAPKLLISLA | | |
| | SDLASGVPSRFSGSESGTDYTLTISSLQPEDFATYYCQ | | |
| | GYFADSGSSGRSWAFGQGTKVEIKRTVAAPSVFIFPP | | |
| | SDEQLKSGTASVVCLLNNFYPREAKVQWKVDNALQ | | |
| | SGNSQESVTEQDSKDSTYSLSSTLTLSKADYEKHKVY | | |
| | ACEVTHQGLSSPVTKSFNRGEC | | |
| ROIS-3 | QVSLEESGGDLVQPGGSLRLACVASGFSFSSGCWVC | HCVR | 51 |
| | WVRQAPGKGLEWVSCIYTGDGDTYYANSVRGRFTV | | |
| | SRQNSTTSVYLQMNSLRADDTALYFCASPQPGDITYL | | |
| | GFWGPGTLVTVSS | | |
| | DIHMTQSPSSLSASVGDTVTITCQASQNINSYLSWYQ | LCVR | 52 |
| | QKPGKAPKLLISLASDLASGVPSRFSGSESGTDYTLTI | | |
| | SSLQPEDFATYYCQGYFADSGSSGRSWAFGQGTKVE | | |
| | IKRTV | | |
| | SGCWVC | HCDR1 | 53 |
| | CIYTGDGDTYYANSVRG | HCDR2 | 54 |
| | PQPGDITYLGF | HCDR3 | 55 |
| | QASQNINSYLS | LCDR1 | 56 |
| | LASDLAS | LCDR2 | 57 |
| | QGYFADSGSSGRSWA | LCDR3 | 58 |
| | METGLRWLLLVAVLKGVQQVSLEESGGDLVQPGGS | HC | 59 |
| | LRLACVASGFSFSSGCWVCWVRQAPGKGLEWVSCIY | | |
| | TGDGDTYYANSVRGRFTVSRQNSTTSVYLQMNSLRA | | |
| | DDTALYFCASPQPGDITYLGFWGPGTLVTVSSASTKG | | |
| | PSVFPLAPCSRSTSESTAALGCLVKDYFPEPVTVSWN | | |
| | SGALTSGVHTFPAVLQSSGLYSLSSVVTVPSSSLGTKT | | |
| | YTCNVDHKPSNTKVDKRVESKYGPPCPPCPAPEFLG | | |
| | GPSVFLFPPKPKDTLMISRTPEVTCVVVDVSQEDPEV | | |
| | QFNWYVDGVEVHNAKTKPREEQFNSTYRVVSVLTV | | |
| | LHQDWLNGKEYKCKVSNKGLPSSIEKTISKAKGQPR | | |
| | EPQVYTLPPSQEEMTKNQVSLTCLVKGFYPSDIAVEW | | |
| | ESNGQPENNYKTTPPVLDSDGSFFLYSRLTVDKSRW | | |
| | QEGNVFSCSVMHEALHNHYTQKSLSLSLGK | | |
| | MDTRAPTQLLGLLLLWLPGARCADIHMTQSPSSLSAS | LC | 60 |
| | VGDTVTITCQASQNINSYLSWYQQKPGKAPKLLISLA | | |
| | SDLASGVPSRFSGSESGTDYTLTISSLQPEDFATYYCQ | | |
| | GYFADSGSSGRSWAFGQGTKVEIKRTVAAPSVFIFPP | | |
| | SDEQLKSGTASVVCLLNNFYPREAKVQWKVDNALQ | | |
| | SGNSQESVTEQDSKDSTYSLSSTLTLSKADYEKHKVY | | |
| | ACEVTHQGLSSPVTKSFNRGEC | | |
| | QVSLEESGGDLVQPGGSLRLACVASGFSFSSGCWVC | HCVR | 61 |
| | WVRQAPGKGLEWVSCIYTGDGDTYYANSVRGRFTV | | |
| | SRKNSTTSVYLQMNSLRADDTALYFCASPQPGDITYL | | |
| | GFWGPGTLVTVSS | | |
| | DIHMTQSPSSLSASVGDTVTITCQASQNINSYLSWYQ | LCVR | 62 |
| | QKPGKAPKLLISLASDLASGVPSRFSGSESGTDYTLTI | | |
| | SSLQPEDFATYYCQGYFADSGSSGRSWAFGQGTKVE | | |
| | IKRTV | | |
| | SGCWVC | HCDR1 | 63 |
| | CIYTGDGDTYYANSVRG | HCDR2 | 64 |
| | PQPGDITYLGF | HCDR3 | 65 |
| | QASQNINSYLS | LCDR1 | 66 |
| | LASDLAS | LCDR2 | 67 |
| | QGYFADSGSSGRSWA | LCDR3 | 68 |
| | METGLRWLLLVAVLKGVQQVSLEESGGDLVQPGGS | HC | 69 |
| | LRLACVASGFSFSSGCWVCWVRQAPGKGLEWVSCIY | | |
| | TGDGDTYYANSVRGRFTVSRKNSTTSVYLQMNSLRA | | |
| | DDTALYFCASPQPGDITYLGFWGPGTLVTVSSASTKG | | |
| | PSVFPLAPCSRSTSESTAALGCLVKDYFPEPVTVSWN | | |
| | SGALTSGVHTFPAVLQSSGLYSLSSVVTVPSSSLGTKT | | |
| | YTCNVDHKPSNTKVDKRVESKYGPPCPPCPAPEFLG | | |
| | GPSVFLFPPKPKDTLMISRTPEVTCVVVDVSQEDPEV | | |
| | QFNWYVDGVEVHNAKTKPREEQFNSTYRVVSVLTV | | |
| | LHQDWLNGKEYKCKVSNKGLPSSIEKTISKAKGQPR | | |
| | EPQVYTLPPSQEEMTKNQVSLTCLVKGFYPSDIAVEW | | |
| | ESNGQPENNYKTTPPVLDSDGSFFLYSRLTVDKSRW | | |
| | QEGNVFSCSVMHEALHNHYTQKSLSLSLGK | | |
| | MDTRAPTQLLGLLLLWLPGARCADIHMTQSPSSLSAS | LC | 70 |
| | VGDTVTITCQASQNINSYLSWYQQKPGKAPKLLISLA | | |
| | SDLASGVPSRFSGSESGTDYTLTISSLQPEDFATYYCQ | | |
| | GYFADSGSSGRSWAFGQGTKVEIKRTVAAPSVFIFPP | | |
| | SDEQLKSGTASVVCLLNNFYPREAKVQWKVDNALQ | | |
| | SGNSQESVTEQDSKDSTYSLSSTLTLSKADYEKHKVY | | |
| | ACEVTHQGLSSPVTKSFNRGEC | | |
| ROIS-5 | QVSLEESGGDLVQPGGSLRLACVASGFSFSSGCWVC | HCVR | 71 |
| | WVRQAPGKGLEWVSCIYTGDGDTYYANSVRGRFTV | | |
| | SRRNSTTSVYLQMNSLRADDTALYFCASPQPGDITYL | | |
| | GFWGPGTLVTVSS | | |
| | DIHMTQSPSSLSASVGDTVTITCQASQNINSYLSWYQ | LCVR | 72 |
| | QKPGKAPKLLISLASDLASGVPSRFSGSESGTDYTLTI | | |
| | SSLQPEDFATYYCQGYFADSGSSGRSWAFGQGTKVE | | |
| | IKRTV | | |
| | SGCWVC | HCDR1 | 73 |
| | CIYTGDGDTYYANSVRG | HCDR2 | 74 |
| | PQPGDITYLGF | HCDR3 | 75 |
| | QASQNINSYLS | LCDR1 | 76 |
| | LASDLAS | LCDR2 | 77 |
| | QGYFADSGSSGRSWA | LCDR3 | 78 |
| | METGLRWLLLVAVLKGVQQVSLEESGGDLVQPGGS | HC | 79 |
| | LRLACVASGFSFSSGCWVCWVRQAPGKGLEWVSCIY | | |
| | TGDGDTYYANSVRGRFTVSRRNSTTSVYLQMNSLRA | | |
| | DDTALYFCASPQPGDITYLGFWGPGTLVTVSSASTKG | | |
| | PSVFPLAPCSRSTSESTAALGCLVKDYFPEPVTVSWN | | |
| | SGALTSGVHTFPAVLQSSGLYSLSSVVTVPSSSLGTKT | | |
| | YTCNVDHKPSNTKVDKRVESKYGPPCPPCPAPEFLG | | |
| | GPSVFLFPPKPKDTLMISRTPEVTCVVVDVSQEDPEV | | |
| | QFNWYVDGVEVHNAKTKPREEQFNSTYRVVSVLTV | | |
| | LHQDWLNGKEYKCKVSNKGLPSSIEKTISKAKGQPR | | |
| | EPQVYTLPPSQEEMTKNQVSLTCLVKGFYPSDIAVEW | | |
| | ESNGQPENNYKTTPPVLDSDGSFFLYSRLTVDKSRW | | |
| | QEGNVFSCSVMHEALHNHYTQKSLSLSLGK | | |
| | MDTRAPTQLLGLLLLWLPGARCADIHMTQSPSSLSAS | LC | 80 |
| | VGDTVTITCQASQNINSYLSWYQQKPGKAPKLLISLA | | |
| | SDLASGVPSRFSGSESGTDYTLTISSLQPEDFATYYCQ | | |
| | GYFADSGSSGRSWAFGQGTKVEIKRTVAAPSVFIFPP | | |
| | SDEQLKSGTASVVCLLNNFYPREAKVQWKVDNALQ | | |
| | SGNSQESVTEQDSKDSTYSLSSTLTLSKADYEKHKVY | | |
| | ACEVTHQGLSSPVTKSFNRGEC | | |

TABLE 2
Nucleic acid sequences of the representative antibodies

| | | OTHER | SEQ ID |
|---|---|---|---|
| ANTIBODY | SEQUENCE | INFORMATION | NO |
| 6C8 | CAGGAGCAACTGGTGGAGTCTGGAGGGGGTCTGGT | HCVR | 81 |
| | CACGCTTGGGGGATCCCTCAAACTCTCCTGCAAAGC | | |
| | CTCTGGAATCGACTTCAGTATCTATGGCATTAGCTG | | |
| | GGTCCGCCAGGCTCCAGGGAAGGGGCTGGAGTGGA | | |
| | TCGCATATATTTATCCTGATTATAGTACCACAGACT | | |
| | ACGCGAGTTGGGTGAATGGCCGATTCACCATCTCCT | | |
| | TCGACAACGCCCAGAATACGGTGTTTCTGGAAATG | | |
| | ACCAGTCTGACAGCCGCGGACACGGCCACCTATTTC | | |
| | TGTGCAAGGCGTTATAGTGGTTATTATGGCTTGTGG | | |
| | GGCCCAGGCACCCTGGTCACCGTCTCCTCA | | |
| | GCCCTTGTGATGACCCAGACTCCATCCTCCGTGTCT | LCVR | 82 |
| | GCACCTGTGGGAGGCACAGTCACCATCAAGTGCCA | | |
| | GGCCAGTCAGAGCATTAGTAGCTACTTAGCCTGGTA | | |
| | TCAACAGAAACCAGGGCAGCGTCCCAAGCTCCTGA | | |
| | TCTATGGTGCATCCAGTCTGGAGTCTGGGGTCCCAT | | |
| | CGCGGTTCAAAGGCAGTGGATCTGGGACAGAGTAC | | |
| | ACTCTCACCATCAGCGACCTGGAGTGTGACGATGCT | | |
| | GCCACTTACTACTGTCAAGGTGCTCATGCAGGTAGT | | |
| | AGTGCTGATGCTTTCGGCGGAGGGACCGAGGTGGT | | |
| | GGTCAAAGGTGATCCA | | |
| | ATCTATGGCATTAGC | HCDR1 | 83 |
| | TATATTTATCCTGATTATAGTACCACAGACTACGCG | HCDR2 | 84 |
| | AGTTGGGTGAATGGC | | |
| | CGTTATAGTGGTTATTATGGCTTG | HCDR3 | 85 |
| | CAGGCCAGTCAGAGCATTAGTAGCTACTTA | LCDR1 | 86 |
| | GGTGCATCCAGTCTGGAG | LCDR2 | 87 |
| | CAAGGTGCTCATGCAGGTAGTAGTGCTGATGCT | LCDR3 | 88 |
| | ATGGAGACTGGGCTGCGCTGGCTTCTCCTGGTCGCT | HC | 89 |
| | GTGCTCAAAGGTGTCCAGTGTCAGGAGCAACTGGT | | |
| | GGAGTCTGGAGGGGGTCTGGTCACGCTTGGGGGAT | | |
| | CCCTCAAACTCTCCTGCAAAGCCTCTGGAATCGACT | | |
| | TCAGTATCTATGGCATTAGCTGGGTCCGCCAGGCTC | | |
| | CAGGGAAGGGGCTGGAGTGGATCGCATATATTTAT | | |
| | CCTGATTATAGTACCACAGACTACGCGAGTTGGGTG | | |
| | AATGGCCGATTCACCATCTCCTTCGACAACGCCCAG | | |
| | AATACGGTGTTTCTGGAAATGACCAGTCTGACAGCC | | |
| | GCGGACACGGCCACCTATTTCTGTGCAAGGCGTTAT | | |
| | AGTGGTTATTATGGCTTGTGGGGCCCAGGCACCCTG | | |
| | GTCACCGTCTCCTCAGGGCAACCTAAGGCTCCATCA | | |
| | GTCTTCCCACTGGCCCCCTGCTGCGGGGACACACCC | | |
| | AGCTCCACGGTGACCCTGGGCTGCCTGGTCAAAGG | | |
| | CTACCTCCCGGAGCCAGTGACCGTGACCTGGAACTC | | |
| | GGGCACCCTCACCAATGGGGTACGCACCTTCCCGTC | | |
| | CGTCCGGCAGTCCTCAGGCCTCTACTCGCTGAGCAG | | |
| | CGTGGTGAGCGTGACCTCAAGCAGCCAGCCCGTCA | | |
| | CCTGCAACGTGGCCCACCCAGCCACCAACACCAAA | | |
| | GTGGACAAGACCGTTGCGCCCTCGACATGCAGCAA | | |
| | GCCCATGTGCCCACCCCCTGAACTCCCGGGGGGAC | | |
| | CGTCTGTCTTCATCTTCCCCCCAAAACCCAAGGACA | | |
| | CCCTCATGATCTCACGCACCCCCGAGGTCACATGCG | | |
| | TGGTGGTGGACGTGAGCCAGGATGACCCCGAGGTG | | |
| | CAGTTCACATGGTACATAAACAACGAGCAGGTGCG | | |
| | CACCGCCCGGCCGCCGCTACGGGAGCAGCAGTTCA | | |
| | ACAGCACGATCCGCGTGGTCAGCACCCTCCCCATCG | | |
| | CGCACCAGGACTGGCTGAGGGGCAAGGAGTTCAAG | | |
| | TGCAAAGTCCACAACAAGGCACTCCCGGCCCCCAT | | |
| | CGAGAAAACCATCTCCAAAGCCAGAGGGCAGCCCC | | |
| | TGGAGCCGAAGGTCTACACCATGGGCCCTCCCCGG | | |
| | GAGGAGCTGAGCAGCAGGTCGGTCAGCCTGACCTG | | |
| | CATGATCAACGGCTTCTACCCTTCCGACATCTCGGT | | |
| | GGAGTGGGAGAAGAACGGGAAGGCAGAGGACAAC | | |
| | TACAAGACCACGCCGACCGTGCTGGACAGCGACGG | | |
| | CTCCTACTTCCTCTACAGCAAGCTCTCAGTGCCCAC | | |
| | GAGTGAGTGGCAGCGGGGCGACGTCTTCACCTGCT | | |
| | CCGTGATGCACGAGGCCTTGCACAACCACTACACG | | |
| | CAGAAGTCCATCTCCCGCTCTCCGGGTAAATAG | | |
| | ATGGACACGAGGGCCCCCACTCAGCTGCTGGGGCT | LC | 90 |
| | CCTGCTGCTCTGGCTCCCAGGTGCCCGATGTGCCCT | | |
| | TGTGATGACCCAGACTCCATCCTCCGTGTCTGCACC | | |
| | TGTGGGAGGCACAGTCACCATCAAGTGCCAGGCCA | | |
| | GTCAGAGCATTAGTAGCTACTTAGCCTGGTATCAAC | | |
| | AGAAACCAGGGCAGCGTCCCAAGCTCCTGATCTAT | | |
| | GGTGCATCCAGTCTGGAGTCTGGGGTCCCATCGCGG | | |
| | TTCAAAGGCAGTGGATCTGGGACAGAGTACACTCT | | |
| | CACCATCAGCGACCTGGAGTGTGACGATGCTGCCA | | |
| | CTTACTACTGTCAAGGTGCTCATGCAGGTAGTAGTG | | |
| | CTGATGCTTTCGGCGGAGGGACCGAGGTGGTGGTC | | |
| | AAAGGTGATCCAGTTGCACCTACTGTCCTCATCTTC | | |
| | CCACCAGCTGCTGATCAGGTGGCAACTGGAACAGT | | |
| | CACCATCGTGTGTGTGGCGAATAAATACTTTCCCGA | | |
| | TGTCACCGTCACCTGGGAGGTGGATGGCACCACCC | | |
| | AAACAACTGGCATCGAGAACAGTAAAACACCGCAG | | |
| | AATTCTGCAGATTGTACCTACAACCTCAGCAGCACT | | |
| | CTGACACTGACCAGCACACAGTACAACAGCCACAA | | |
| | AGAGTACACCTGCAAGGTGACCCAGGGCACGACCT | | |
| | CAGTCGTCCAGAGCTTCAATAGGGGTGACTGTTAG | | |
| 8H6 | TGTCAGTCGTTGGAGGAGTCCGGGGGAGACCTGGT | HCVR | 91 |
| | CAAGCCTGAGGGATCCCTGACACTCACCTGCAAAG | | |
| | CCTCTGGATTCTCCTTCAGTAGCGGTTGCTGGGTAT | | |
| | GCTGGGTCCGCCAGGCTCCAGGGAAGGGGTTGGAG | | |
| | TGGATCGCATGTATTTATACTGGTGATGGTGACACT | | |
| | TACTACGCGAACTGGGCGAGAGGCCGATTCACCGT | | |
| | CTCCCGTACCTCGTCGACCACGGTGACTCTGCAATT | | |
| | GACCAGTCTGACAGCCGCGGACACGGCCACCTATT | | |
| | TCTGTGCGAGTCCACAGCCTGGTGACATTACTTATT | | |
| | TGGGGTTCTGGGGCCCGGGCACCCTGGTCACCGTCT | | |
| | CCTCA | | |
| | GACATTGTGATGACCCAGACTCCAGCCTCCGTGTCT | LCVR | 92 |
| | GAACCTGTGGGAGGCACAGTCACCATCAAGTGCCA | | |
| | GGCCAGTCAGAACATTAATAGCTACTTATCCTGGTA | | |
| | TCAGCAGAGACCAGGGCAGCCTCCCAAGCTCCTGA | | |
| | TCTCACTGGCATCCGATCTGGCATCTGGGGTCCCAT | | |
| | CGCGGTTTAAAGGCAGTGAATCTGGGACAGACTAT | | |
| | ACTCTCACCATCAGCGACCTGGAGTGTGCCGATGCT | | |
| | GCCACTTACTACTGTCAAGGCTATTTTGCTGATAGT | | |
| | GGTAGTAGTGGTCGTTCGTGGGCTTTCGGCGGCGGG | | |
| | ACCGAGGTGGTGGTCCAAGGTGATCCA | | |
| | AGCGGTTGCTGGGTATGC | HCDR1 | 93 |
| | TGTATTTATACTGGTGATGGTGACACTTACTACGCG | HCDR2 | 94 |
| | AACTGGGCGAGAGGC | | |
| | CCACAGCCTGGTGACATTACTTATTTGGGGTTC | HCDR3 | 95 |
| | CAGGCCAGTCAGAACATTAATAGCTACTTATCC | LCDR1 | 96 |
| | CTGGCATCCGATCTGGCATCT | LCDR2 | 97 |
| | CAAGGCTATTTTGCTGATAGTGGTAGTAGTGGTCGT | LCDR3 | 98 |
| | TCGTGGGCT | | |
| | ATGGAGACTGGGCTGCGCTGGCTTCTCCTGGTCGCT | HC | 99 |
| | GTGCTCAAAGGTGTCCAGTGTCAGTCGTTGGAGGA | | |
| | GTCCGGGGGAGACCTGGTCAAGCCTGAGGGATCCC | | |
| | TGACACTCACCTGCAAAGCCTCTGGATTCTCCTTCA | | |
| | GTAGCGGTTGCTGGGTATGCTGGGTCCGCCAGGCTC | | |
| | CAGGGAAGGGGTTGGAGTGGATCGCATGTATTTAT | | |
| | ACTGGTGATGGTGACACTTACTACGCGAACTGGGC | | |
| | GAGAGGCCGATTCACCGTCTCCCGTACCTCGTCGAC | | |
| | CACGGTGACTCTGCAATTGACCAGTCTGACAGCCGC | | |
| | GGACACGGCCACCTATTTCTGTGCGAGTCCACAGCC | | |
| | TGGTGACATTACTTATTTGGGGTTCTGGGGCCCGGG | | |
| | CACCCTGGTCACCGTCTCCTCAGGGCAACCTAAGGC | | |
| | TCCATCAGTCTTCCCACTGGCCCCCTGCTGCGGGGA | | |
| | CACACCCAGCTCCACGGTGACCCTGGGCTGCCTGGT | | |
| | CAAAGGCTACCTCCCGGAGCCAGTGACCGTGACCT | | |
| | GGAACTCGGGCACCCTCACCAATGGGGTACGCACC | | |
| | TTCCCGTCCGTCCGGCAGTCCTCAGGCCTCTACTCG | | |
| | CTGAGCAGCGTGGTGAGCGTGACCTCAAGCAGCCA | LC | 100 |
| | GCCCGTCACCTGCAACGTGGCCCACCCAGCCACCA | | |
| | ACACCAAAGTGGACAAGACCGTTGCGCCCTCGACA | | |
| | TGCAGCAAGCCCATGTGCCCACCCCCTGAACTCCCG | | |
| | GGGGGACCGTCTGTCTTCATCTTCCCCCCAAAACCC | | |
| | AAGGACACCCTCATGATCTCACGCACCCCCGAGGT | | |
| | CACATGCGTGGTGGTGGACGTGAGCCAGGATGACC | | |
| | CCGAGGTGCAGTTCACATGGTACATAAACAACGAG | | |
| | CAGGTGCGCACCGCCCGGCCGCCGCTACGGGAGCA | | |
| | GCAGTTCAACAGCACGATCCGCGTGGTCAGCACCC | | |
| | TCCCCATCGCGCACCAGGACTGGCTGAGGGGCAAG | | |
| | GAGTTCAAGTGCAAAGTCCACAACAAGGCACTCCC | | |
| | GGCCCCCATCGAGAAAACCATCTCCAAAGCCAGAG | | |
| | GGCAGCCCCTGGAGCCGAAGGTCTACACCATGGGC | | |
| | CCTCCCCGGGAGGAGCTGAGCAGCAGGTCGGTCAG | | |
| | CCTGACCTGCATGATCAACGGCTTCTACCCTTCCGA | | |
| | CATCTCGGTGGAGTGGGAGAAGAACGGGAAGGCAG | | |
| | AGGACAACTACAAGACCACGCCGACCGTGCTGGAC | | |
| | AGCGACGGCTCCTACTTCCTCTACAGCAAGCTCTCA | | |
| | GTGCCCACGAGTGAGTGGCAGCGGGGCGACGTCTT | | |
| | CACCTGCTCCGTGATGCACGAGGCCTTGCACAACCA | | |
| | CTACACGCAGAAGTCCATCTCCCGCTCTCCGGGTAA | | |
| | ATAG | | |
| | ATGGACACGAGGGCCCCCACTCAGCTGCTGGGGCT | | |
| | CCTGCTGCTCTGGCTCCCAGGTGCCAGATGTGCTGA | | |
| | CATTGTGATGACCCAGACTCCAGCCTCCGTGTCTGA | | |
| | ACCTGTGGGAGGCACAGTCACCATCAAGTGCCAGG | | |
| | CCAGTCAGAACATTAATAGCTACTTATCCTGGTATC | | |
| | AGCAGAGACCAGGGCAGCCTCCCAAGCTCCTGATC | | |
| | TCACTGGCATCCGATCTGGCATCTGGGGTCCCATCG | | |
| | CGGTTTAAAGGCAGTGAATCTGGGACAGACTATAC | | |
| | TCTCACCATCAGCGACCTGGAGTGTGCCGATGCTGC | | |
| | CACTTACTACTGTCAAGGCTATTTTGCTGATAGTGG | | |
| | TAGTAGTGGTCGTTCGTGGGCTTTCGGCGGCGGGAC | | |
| | CGAGGTGGTGGTCCAAGGTGATCCAGTTGCACCTA | | |
| | CTGTCCTCATCTTCCCACCAGCTACTGATCAGGTGG | | |
| | CAACTGGAACAGTCACCATCGTGTGTGTGGCGAAT | | |
| | AAATACTTTCCCGATGTCACCGTCACCTGGGAGGTG | | |
| | GATGGCACCACCCAAACAACTGGCATCGAGAACAG | | |
| | TAAAACACCGCAGAATTCTGCAGATTGTACCTACA | | |
| | ACCTCAGCAGCACTCTGACACTGACCAGCACACAG | | |
| | TACAACAGCCACAAAGAGTACACCTGCAAGGTGAC | | |
| | CCAGGGCACGACCTCAGTCGTCCAGAGCTTCAATA | | |
| | GGGGTGACTGTTAG | | |
| 14G9 | CAGGAGCAGCTGGTGGAGTCCGGGGGAGGCCTGGT | HCVR | 101 |
| | CCAGCCTGAGGGATCCCTGACACTCACCTGCACAG | | |
| | CTTCTGGATTCTCCTTCAGTAGCGACTACTACATGT | | |
| | GCTGGGTCCGCCAGGCTCCAGGGAAGGGGCTGGAG | | |
| | TGGATCGCTTGCATTGGTGGTGGTTATGGTGGTAGC | | |
| | CCTTACTACGCGACCTGGGCGAAAGGCCGCTTCACC | | |
| | ATCTCCAAAACCTCGTCGACCGCGGTGACTCTACAA | | |
| | ATGACCAGTCTGACAGCCGCGGACACGGCCACCCA | | |
| | TTTCTGTGCGCGCGGTGTGCCTGGTAGTAGTTGTTT | | |
| | CTTTGACTTGTGGGGCCCAGGCACCCTGGTCACCGT | | |
| | CTCCTCA | | |
| | GATGTTGCGATGACCCAGACTCCAGCCTCCGTGTCT | LCVR | 102 |
| | GAACCTGTGGGAGGCACAGTCACCATCAAGTGCCA | | |
| | GGCCAGTCAGAGCATTGGTAGTAGCTTAGCCTGGT | | |
| | ATCAACAAAAATCAGGACAGCCTCCCAAGCTCCTG | | |
| | ATCTATGATGCATTTGATCTGGCATCTGGAGTCTCA | | |
| | TCGCGGTTCAAAGGCAGTGGATCTGGGACAGAGTA | | |
| | CACTCTCACCATCAGCGACCTGGAGTGTGCCGATGC | | |
| | TGCCACTTATTTCTGTCTAGGCGGCTATTATGATGC | | |
| | TAATACTTTTGGCGGAGGGACCGAGGTGGTGGTCA | | |
| | AAGGTGATCCA | | |
| | AGCGACTACTACATGTGC | HCDR1 | 103 |
| | TGCATTGGTGGTGGTTATGGTGGTAGCCCTTACTAC | HCDR2 | 104 |
| | GCGACCTGGGCGAAAGGC | | |
| | GGTGTGCCTGGTAGTAGTTGTTTCTTTGACTTG | HCDR3 | 105 |
| | CAGGCCAGTCAGAGCATTGGTAGTAGCTTAGCC | LCDR1 | 106 |
| | GATGCATTTGATCTGGCATCT | LCDR2 | 107 |
| | CTAGGCGGCTATTATGATGCTAATACT | LCDR3 | 108 |
| | ATGGAGACTGGGCTGCGCTGGCTTCTCCTGGTCGCT | HC | 109 |
| | GTGCTCAAAGGTGTCCAGTGTCAGGAGCAGCTGGT | | |
| | GGAGTCCGGGGGAGGCCTGGTCCAGCCTGAGGGAT | | |
| | CCCTGACACTCACCTGCACAGCTTCTGGATTCTCCT | | |
| | TCAGTAGCGACTACTACATGTGCTGGGTCCGCCAGG | | |
| | CTCCAGGGAAGGGGCTGGAGTGGATCGCTTGCATT | | |
| | GGTGGTGGTTATGGTGGTAGCCCTTACTACGCGACC | | |
| | TGGGCGAAAGGCCGCTTCACCATCTCCAAAACCTC | | |
| | GTCGACCGCGGTGACTCTACAAATGACCAGTCTGA | | |
| | CAGCCGCGGACACGGCCACCCATTTCTGTGCGCGC | | |
| | GGTGTGCCTGGTAGTAGTTGTTTCTTTGACTTGTGG | | |
| | GGCCCAGGCACCCTGGTCACCGTCTCCTCAGGGCA | | |
| | ACCTAAGGCTCCATCAGTCTTCCCACTGGCCCCCTG | | |
| | CTGCGGGGACACACCCAGCTCCACGGTGACCCTGG | | |
| | GCTGCCTGGTCAAAGGCTACCTCCCGGAGCCAGTG | | |
| | ACCGTGACCTGGAACTCGGGCACCCTCACCAATGG | | |
| | GGTACGCACCTTCCCGTCCGTCCGGCAGTCCTCAGG | | |
| | CCTCTACTCGCTGAGCAGCGTGGTGAGCGTGACCTC | | |
| | AAGCAGCCAGCCCGTCACCTGCAACGTGGCCCACC | | |
| | CAGCCACCAACACCAAAGTGGACAAGACCGTTGCG | | |
| | CCCTCGACATGCAGCAAGCCCATGTGCCCACCCCCT | LC | 110 |
| | GAACTCCCGGGGGGACCGTCTGTCTTCATCTTCCCC | | |
| | CCAAAACCCAAGGACACCCTCATGATCTCACGCAC | | |
| | CCCCGAGGTCACATGCGTGGTGGTGGACGTGAGCC | | |
| | AGGATGACCCCGAGGTGCAGTTCACATGGTACATA | | |
| | AACAACGAGCAGGTGCGCACCGCCCGGCCGCCGCT | | |
| | ACGGGAGCAGCAGTTCAACAGCACGATCCGCGTGG | | |
| | TCAGCACCCTCCCCATCGCGCACCAGGACTGGCTGA | | |
| | GGGGCAAGGAGTTCAAGTGCAAAGTCCACAACAAG | | |
| | GCACTCCCGGCCCCCATCGAGAAAACCATCTCCAA | | |
| | AGCCAGAGGGCAGCCCCTGGAGCCGAAGGTCTACA | | |
| | CCATGGGCCCTCCCCGGGAGGAGCTGAGCAGCAGG | | |
| | TCGGTCAGCCTGACCTGCATGATCAACGGCTTCTAC | | |
| | CCTTCCGACATCTCGGTGGAGTGGGAGAAGAACGG | | |
| | GAAGGCAGAGGACAACTACAAGACCACGCCGACCG | | |
| | TGCTGGACAGCGACGGCTCCTACTTCCTCTACAGCA | | |
| | AGCTCTCAGTGCCCACGAGTGAGTGGCAGCGGGGC | | |
| | GACGTCTTCACCTGCTCCGTGATGCACGAGGCCTTG | | |
| | CACAACCACTACACGCAGAAGTCCATCTCCCGCTCT | | |
| | CCGGGTAAATAG | | |
| | ATGGACACGAGGGCCCCCACTCAGCTGCTGGGGCT | | |
| | CCTGCTGCTCTGGCTCCCAGGTGCCAGCTGTGATGT | | |
| | TGCGATGACCCAGACTCCAGCCTCCGTGTCTGAACC | | |
| | TGTGGGAGGCACAGTCACCATCAAGTGCCAGGCCA | | |
| | GTCAGAGCATTGGTAGTAGCTTAGCCTGGTATCAAC | | |
| | AAAAATCAGGACAGCCTCCCAAGCTCCTGATCTAT | | |
| | GATGCATTTGATCTGGCATCTGGAGTCTCATCGCGG | | |
| | TTCAAAGGCAGTGGATCTGGGACAGAGTACACTCT | | |
| | CACCATCAGCGACCTGGAGTGTGCCGATGCTGCCA | | |
| | CTTATTTCTGTCTAGGCGGCTATTATGATGCTAATA | | |
| | CTTTTGGCGGAGGGACCGAGGTGGTGGTCAAAGGT | | |
| | GATCCAGTTGCACCTACTGTCCTCATCTTCCCACCA | | |
| | GCTGCTGATCAGGTGGCAACTGGAACAGTCACCAT | | |
| | CGTGTGTGTGGCGAATAAATACTTTCCCGATGTCAC | | |
| | CGTCACCTGGGAGGTGGATGGCACCACCCAAACAA | | |
| | CTGGCATCGAGAACAGTAAAACACCGCAGAATTCT | | |
| | GCAGATTGTACCTACAACCTCAGCAGCACTCTGACA | | |
| | CTGACCAGCACACAGTACAACAGCCACAAAGAGTA | | |
| | CACCTGCAAGGTGACCCAGGGCACGACCTCAGTCG | | |
| | TCCAGAGCTTCAATAGGGGTGACTGTTAG | | |
| ROIS-1 | AACGTGCAGCTGGTCGAGTCTGGAGGCGGCCTGAT | HCVR | 111 |
| | CCACCCCGGCGGAAGCCTCTCTCTGTCCTGCAAGGC | | |
| | CTCTGGCTTTTCTTTCTCCTCCGGCTGTTGGGTGTGC | | |
| | TGGGTGCGGCAAGCTCCTGGAAAAGGCCTGGAATG | | |
| | GGTCGCCTGCATCTACACCGGCGACGGCGATACAT | | |
| | ATTACGCCGACTCCGTGAGAGGCAGATTCACCATCT | | |
| | CCCGGACCTCTTCCAAGACCACCGTGTACCTGCAGA | | |
| | TGAACTCCCTGAGAGCCGAGGACACCGCCGTGTAC | | |
| | TACTGCGCTTCTCCTCAGCCTGGCGACATCACCTAC | | |
| | CTGGGCTTCTGGGGCCAGGGCACACTGGTGACCGT | | |
| | GTCCAGC | | |
| | GACATCCACATGACCCAGTCTCCAAGCAGCCTGTCC | LCVR | 112 |
| | GCTTCTGTCGGCGACACCGTGACAATCACCTGTCAG | | |
| | GCCTCTCAGAACATCAACTCCTACCTGTCTTGGTAT | | |
| | CAGCAAGAGCCCGGCAAGGCCCCTAAGCTGCTGAT | | |
| | CTCTCTGGCTTCCGACCTGGCCAGCGGCGTGCCTTC | | |
| | TCGGTTCTCCGGATCCGAGTCCGGCACCGACTACAC | | |
| | CCTGACCATCTCCTCCCTGCAGCCTGAGGATTTTGC | | |
| | TACCTACTACTGCCAGGGCTACTTCGCCGATTCTGG | | |
| | CTCCTCTGGCAGATCCTGGGCCTTCGGCCAGGGCAC | | |
| | AAAAGTGGAAATCAAGAGAACCGTG | | |
| | TCCGGCTGTTGGGTGTGC | HCDR1 | 113 |
| | TGCATCTACACCGGCGACGGCGATACATATTACGCC | HCDR2 | 114 |
| | GACTCCGTGAGAGGC | | |
| | CCTCAGCCTGGCGACATCACCTACCTGGGCTTC | HCDR3 | 115 |
| | CAGGCCTCTCAGAACATCAACTCCTACCTGTCT | LCDR1 | 116 |
| | CTGGCTTCCGACCTGGCCAGC | LCDR2 | 117 |
| | CAGGGCTACTTCGCCGATTCTGGCTCCTCTGGCAGA | LCDR3 | 118 |
| | TCCTGGGCC | | |
| | ATGGAAACTGGTCTACGATGGCTACTACTAGTGGCT | HC | 119 |
| | GTGCTGAAGGGCGTGCAGAACGTGCAGCTGGTCGA | | |
| | GTCTGGAGGCGGCCTGATCCACCCCGGCGGAAGCC | | |
| | TCTCTCTGTCCTGCAAGGCCTCTGGCTTTTCTTTCTC | | |
| | CTCCGGCTGTTGGGTGTGCTGGGTGCGGCAAGCTCC | | |
| | TGGAAAAGGCCTGGAATGGGTCGCCTGCATCTACA | | |
| | CCGGCGACGGCGATACATATTACGCCGACTCCGTG | | |
| | AGAGGCAGATTCACCATCTCCCGGACCTCTTCCAAG | | |
| | ACCACCGTGTACCTGCAGATGAACTCCCTGAGAGC | | |
| | CGAGGACACCGCCGTGTACTACTGCGCTTCTCCTCA | | |
| | GCCTGGCGACATCACCTACCTGGGCTTCTGGGGCCA | | |
| | GGGCACACTGGTGACCGTGTCCAGCGCCTCTACCA | | |
| | AGGGCCCTTCTGTGTTCCCTCTGGCCCCTTGCTCCC | | |
| | GGTCCACCTCCGAGTCTACAGCTGCTCTGGGCTGCC | | |
| | TGGTGAAGGACTACTTCCCCGAGCCCGTGACCGTGT | | |
| | CTTGGAACTCCGGCGCTCTGACATCTGGCGTGCACA | | |
| | CCTTCCCTGCTGTGCTGCAGTCCTCTGGACTGTACT | | |
| | CCCTGTCCAGCGTGGTGACCGTGCCTTCTTCTAGCC | | |
| | TCGGCACCAAAACCTACACCTGCAACGTGGACCAC | | |
| | AAGCCTTCCAACACCAAAGTGGATAAGCGGGTGGA | | |
| | ATCTAAGTACGGCCCCCCCTGCCCTCCTTGTCCTGC | | |
| | TCCTGAGTTCCTGGGAGGACCTTCCGTGTTTCTGTTT | | |
| | CCTCCTAAGCCCAAGGACACCCTGATGATCTCTCGG | | |
| | ACCCCTGAAGTGACCTGTGTGGTCGTGGACGTGTCC | LC | 120 |
| | CAAGAGGACCCTGAGGTGCAGTTCAACTGGTACGT | | |
| | GGATGGCGTGGAAGTGCACAACGCCAAGACCAAGC | | |
| | CTAGAGAAGAGCAGTTCAACTCTACCTACAGAGTG | | |
| | GTGTCCGTGCTGACCGTGCTGCACCAGGACTGGCTG | | |
| | AACGGCAAAGAGTACAAGTGCAAGGTGTCTAACAA | | |
| | GGGCCTGCCTTCCTCCATCGAGAAGACCATCTCCAA | | |
| | GGCCAAGGGCCAACCCAGAGAGCCTCAGGTGTACA | | |
| | CACTGCCTCCAAGCCAAGAGGAAATGACCAAGAAC | | |
| | CAGGTGTCCCTGACCTGCCTGGTCAAGGGCTTCTAC | | |
| | CCTAGCGACATCGCCGTGGAATGGGAGTCCAATGG | | |
| | CCAGCCTGAGAACAACTACAAGACCACCCCACCTG | | |
| | TGCTGGACTCCGACGGCTCCTTCTTCCTGTACTCTA | | |
| | GACTGACAGTGGACAAGTCCAGATGGCAGGAGGGC | | |
| | AACGTGTTCTCCTGTTCTGTGATGCACGAGGCCCTG | | |
| | CACAACCACTACACCCAGAAGTCTCTGTCTCTGTCT | | |
| | CTGGGCAAGTGA | | |
| | ATGGATACTCGAGCACCAACTCAACTACTAGGACT | | |
| | GCTGCTCCTGTGGCTGCCTGGCGCTAGATGCGCCGA | | |
| | CATCCACATGACCCAGTCTCCAAGCAGCCTGTCCGC | | |
| | TTCTGTCGGCGACACCGTGACAATCACCTGTCAGGC | | |
| | CTCTCAGAACATCAACTCCTACCTGTCTTGGTATCA | | |
| | GCAAGAGCCCGGCAAGGCCCCTAAGCTGCTGATCT | | |
| | CTCTGGCTTCCGACCTGGCCAGCGGCGTGCCTTCTC | | |
| | GGTTCTCCGGATCCGAGTCCGGCACCGACTACACCC | | |
| | TGACCATCTCCTCCCTGCAGCCTGAGGATTTTGCTA | | |
| | CCTACTACTGCCAGGGCTACTTCGCCGATTCTGGCT | | |
| | CCTCTGGCAGATCCTGGGCCTTCGGCCAGGGCACA | | |
| | AAAGTGGAAATCAAGAGAACCGTGGCCGCTCCTTC | | |
| | TGTGTTCATCTTCCCTCCTTCCGACGAGCAGCTGAA | | |
| | GTCCGGCACCGCTTCCGTGGTGTGCCTGCTGAACAA | | |
| | CTTCTACCCTAGAGAGGCCAAGGTGCAGTGGAAGG | | |
| | TGGACAACGCCCTGCAGTCTGGCAACTCCCAAGAG | | |
| | TCCGTCACCGAGCAGGACTCCAAGGACTCTACCTAC | | |
| | TCCCTGTCCTCCACCCTGACACTGAGCAAGGCCGAC | | |
| | TACGAGAAGCACAAGGTGTACGCCTGCGAGGTGAC | | |
| | CCACCAGGGCCTGTCTTCTCCTGTGACCAAGTCCTT | | |
| | CAACAGAGGCGAGTGCTGA | | |
| ROIS-2 | AACGTGCAGCTGGTCGAGTCTGGCGGAGGACTGAT | HCVR | 121 |
| | CCACCCCGGCGGCTCTCTGAGCCTCTCCTGCAAGGC | | |
| | CTCTGGATTTAGCTTCTCCTCCGGCTGTTGGGTGTG | | |
| | CTGGGTGCGGCAAGCTCCTGGCAAGGGCCTGGAAT | | |
| | GGGTCGCCTGCATCTACACCGGCGATGGCGACACC | | |
| | TACTACGCCGACTCCGTGAAAGGCAGATTCACCATC | | |
| | TCCCGGACATCTTCCAAGACCACCGTGTACCTGCAG | | |
| | ATGAACTCCCTGAGAGCCGAGGACACAGCCGTGTA | | |
| | CTACTGCGCTTCTCCTCAGCCTGGCGACATCACCTA | | |
| | TCTGGGCTTCTGGGGCCAGGGCACCCTGGTGACCGT | | |
| | GTCCTCT | | |
| | GACATCCACATGACCCAGTCCCCATCCAGCCTGTCT | LCVR | 122 |
| | GCTTCCGTGGGCGACACCGTCACAATCACCTGTCAG | | |
| | GCCTCTCAGAACATCAACTCCTACCTGTCCTGGTAT | | |
| | CAGCAGAAACCCGGCAAGGCCCCTAAGCTGCTGAT | | |
| | CTCCCTGGCCTCCGACCTGGCTTCTGGCGTGCCTTC | | |
| | TCGGTTCTCCGGCTCCGAGTCCGGCACCGACTACAC | | |
| | CCTGACCATCAGCTCTCTGCAGCCTGAGGATTTTGC | | |
| | TACCTACTACTGCCAGGGCTACTTCGCCGATTCTGG | | |
| | CTCTAGCGGCAGATCTTGGGCCTTCGGCCAAGGAA | | |
| | CAAAGGTGGAAATCAAGAGAACCGTG | | |
| | TCCGGCTGTTGGGTGTGC | HCDR1 | 123 |
| | TGCATCTACACCGGCGATGGCGACACCTACTACGCC | HCDR2 | 124 |
| | GACTCCGTGAAAGGC | | |
| | CCTCAGCCTGGCGACATCACCTATCTGGGCTTC | HCDR3 | 125 |
| | CAGGCCTCTCAGAACATCAACTCCTACCTGTCC | LCDR1 | 126 |
| | CTGGCCTCCGACCTGGCTTCT | LCDR2 | 127 |
| | CAGGGCTACTTCGCCGATTCTGGCTCTAGCGGCAGA | LCDR3 | 128 |
| | TCTTGGGCC | | |
| | ATGGAAACTGGTCTACGATGGCTACTACTAGTGGCT | HC | 129 |
| | GTGCTGAAGGGCGTGCAGAACGTGCAGCTGGTCGA | | |
| | GTCTGGCGGAGGACTGATCCACCCCGGCGGCTCTCT | | |
| | GAGCCTCTCCTGCAAGGCCTCTGGATTTAGCTTCTC | | |
| | CTCCGGCTGTTGGGTGTGCTGGGTGCGGCAAGCTCC | | |
| | TGGCAAGGGCCTGGAATGGGTCGCCTGCATCTACA | | |
| | CCGGCGATGGCGACACCTACTACGCCGACTCCGTG | | |
| | AAAGGCAGATTCACCATCTCCCGGACATCTTCCAAG | | |
| | ACCACCGTGTACCTGCAGATGAACTCCCTGAGAGC | | |
| | CGAGGACACAGCCGTGTACTACTGCGCTTCTCCTCA | | |
| | GCCTGGCGACATCACCTATCTGGGCTTCTGGGGCCA | | |
| | GGGCACCCTGGTGACCGTGTCCTCTGCCTCTACCAA | | |
| | GGGCCCTTCTGTGTTCCCTCTGGCCCCTTGCTCCCG | | |
| | GTCCACCTCCGAGTCTACAGCTGCTCTGGGCTGCCT | | |
| | GGTGAAGGACTACTTCCCCGAGCCCGTGACCGTGTC | | |
| | TTGGAACTCCGGCGCTCTGACATCTGGCGTGCACAC | | |
| | CTTCCCTGCTGTGCTGCAGTCCTCTGGACTGTACTC | | |
| | CCTGTCCAGCGTGGTGACCGTGCCTTCTTCTAGCCT | | |
| | CGGCACCAAAACCTACACCTGCAACGTGGACCACA | | |
| | AGCCTTCCAACACCAAAGTGGATAAGCGGGTGGAA | | |
| | TCTAAGTACGGCCCCCCCTGCCCTCCTTGTCCTGCT | | |
| | CCTGAGTTCCTGGGAGGACCTTCCGTGTTTCTGTTT | | |
| | CCTCCTAAGCCCAAGGACACCCTGATGATCTCTCGG | | |
| | ACCCCTGAAGTGACCTGTGTGGTCGTGGACGTGTCC | | |
| | CAAGAGGACCCTGAGGTGCAGTTCAACTGGTACGT | | |
| | GGATGGCGTGGAAGTGCACAACGCCAAGACCAAGC | LC | 130 |
| | CTAGAGAAGAGCAGTTCAACTCTACCTACAGAGTG | | |
| | GTGTCCGTGCTGACCGTGCTGCACCAGGACTGGCTG | | |
| | AACGGCAAAGAGTACAAGTGCAAGGTGTCTAACAA | | |
| | GGGCCTGCCTTCCTCCATCGAGAAGACCATCTCCAA | | |
| | GGCCAAGGGCCAACCCAGAGAGCCTCAGGTGTACA | | |
| | CACTGCCTCCAAGCCAAGAGGAAATGACCAAGAAC | | |
| | CAGGTGTCCCTGACCTGCCTGGTCAAGGGCTTCTAC | | |
| | CCTAGCGACATCGCCGTGGAATGGGAGTCCAATGG | | |
| | CCAGCCTGAGAACAACTACAAGACCACCCCACCTG | | |
| | TGCTGGACTCCGACGGCTCCTTCTTCCTGTACTCTA | | |
| | GACTGACAGTGGACAAGTCCAGATGGCAGGAGGGC | | |
| | AACGTGTTCTCCTGTTCTGTGATGCACGAGGCCCTG | | |
| | CACAACCACTACACCCAGAAGTCTCTGTCTCTGTCT | | |
| | CTGGGCAAGTGA | | |
| | ATGGATACTCGAGCACCAACTCAACTACTAGGACT | | |
| | GCTGCTCCTGTGGCTGCCTGGCGCTAGATGCGCCGA | | |
| | CATCCACATGACCCAGTCCCCATCCAGCCTGTCTGC | | |
| | TTCCGTGGGCGACACCGTCACAATCACCTGTCAGGC | | |
| | CTCTCAGAACATCAACTCCTACCTGTCCTGGTATCA | | |
| | GCAGAAACCCGGCAAGGCCCCTAAGCTGCTGATCT | | |
| | CCCTGGCCTCCGACCTGGCTTCTGGCGTGCCTTCTC | | |
| | GGTTCTCCGGCTCCGAGTCCGGCACCGACTACACCC | | |
| | TGACCATCAGCTCTCTGCAGCCTGAGGATTTTGCTA | | |
| | CCTACTACTGCCAGGGCTACTTCGCCGATTCTGGCT | | |
| | CTAGCGGCAGATCTTGGGCCTTCGGCCAAGGAACA | | |
| | AAGGTGGAAATCAAGAGAACCGTGGCCGCTCCTTC | | |
| | TGTGTTCATCTTCCCTCCTTCCGACGAGCAGCTGAA | | |
| | GTCCGGCACCGCTTCCGTGGTGTGCCTGCTGAACAA | | |
| | CTTCTACCCTAGAGAGGCCAAGGTGCAGTGGAAGG | | |
| | TGGACAACGCCCTGCAGTCTGGCAACTCCCAAGAG | | |
| | TCCGTCACCGAGCAGGACTCCAAGGACTCTACCTAC | | |
| | TCCCTGTCCTCCACCCTGACACTGAGCAAGGCCGAC | | |
| | TACGAGAAGCACAAGGTGTACGCCTGCGAGGTGAC | | |
| | CCACCAGGGCCTGTCTTCTCCTGTGACCAAGTCCTT | | |
| | CAACAGAGGCGAGTGCTGA | | |
| ROIS-3 | CAGGTGTCCCTGGAAGAGTCCGGCGGCGACCTGGT | HCVR | 131 |
| | CCAACCAGGAGGATCTCTCCGCCTGGCCTGCGTGGC | | |
| | TAGCGGCTTTTCCTTCTCCTCTGGCTGTTGGGTGTGC | | |
| | TGGGTCAGACAGGCTCCTGGCAAGGGCCTGGAGTG | | |
| | GGTGTCCTGCATCTACACCGGCGATGGCGACACCTA | | |
| | CTACGCCAACTCCGTGCGGGGCAGATTCACAGTGTC | | |
| | TCGGCAGAACTCCACCACCTCTGTGTACCTGCAGAT | | |
| | GAACTCTCTGAGAGCCGACGACACAGCTCTGTACTT | | |
| | CTGCGCCTCTCCTCAGCCCGGAGATATCACCTATCT | | |
| | GGGCTTCTGGGGCCCTGGCACCCTGGTGACCGTGTC | | |
| | CAGC | | |
| | GACATCCACATGACCCAGTCCCCATCCAGCCTGTCT | LCVR | 132 |
| | GCTTCCGTGGGCGACACCGTCACAATCACCTGTCAG | | |
| | GCCTCTCAGAACATCAACTCCTACCTGTCCTGGTAT | | |
| | CAGCAGAAACCCGGCAAGGCCCCTAAGCTGCTGAT | | |
| | CTCCCTGGCCTCCGACCTGGCTTCTGGCGTGCCTTC | | |
| | TCGGTTCTCCGGCTCCGAGTCCGGCACCGACTACAC | | |
| | CCTGACCATCAGCTCTCTGCAGCCTGAGGATTTTGC | | |
| | TACCTACTACTGCCAGGGCTACTTCGCCGATTCTGG | | |
| | CTCTAGCGGCAGATCTTGGGCCTTCGGCCAAGGAA | | |
| | CAAAGGTGGAAATCAAGAGAACCGTG | | |
| | TCTGGCTGTTGGGTGTGC | HCDR1 | 133 |
| | TGCATCTACACCGGCGATGGCGACACCTACTACGCC | HCDR2 | 134 |
| | AACTCCGTGCGGGGC | | |
| | CCTCAGCCCGGAGATATCACCTATCTGGGCTTC | HCDR3 | 135 |
| | CAGGCCTCTCAGAACATCAACTCCTACCTGTCC | LCDR1 | 136 |
| | CTGGCCTCCGACCTGGCTTCT | LCDR2 | 137 |
| | CAGGGCTACTTCGCCGATTCTGGCTCTAGCGGCAGA | LCDR3 | 138 |
| | TCTTGGGCC | | |
| | ATGGAAACTGGTCTACGATGGCTACTACTAGTGGCC | HC | 139 |
| | GTGCTGAAGGGCGTGCAGCAGGTGTCCCTGGAAGA | | |
| | GTCCGGCGGCGACCTGGTCCAACCAGGAGGATCTC | | |
| | TCCGCCTGGCCTGCGTGGCTAGCGGCTTTTCCTTCT | | |
| | CCTCTGGCTGTTGGGTGTGCTGGGTCAGACAGGCTC | | |
| | CTGGCAAGGGCCTGGAGTGGGTGTCCTGCATCTAC | | |
| | ACCGGCGATGGCGACACCTACTACGCCAACTCCGT | | |
| | GCGGGGCAGATTCACAGTGTCTCGGCAGAACTCCA | | |
| | CCACCTCTGTGTACCTGCAGATGAACTCTCTGAGAG | | |
| | CCGACGACACAGCTCTGTACTTCTGCGCCTCTCCTC | | |
| | AGCCCGGAGATATCACCTATCTGGGCTTCTGGGGCC | | |
| | CTGGCACCCTGGTGACCGTGTCCAGCGCCTCTACCA | | |
| | AGGGCCCTTCTGTGTTCCCTCTGGCCCCTTGCTCCC | | |
| | GGTCCACCTCCGAGTCTACAGCTGCTCTGGGCTGCC | | |
| | TGGTGAAGGACTACTTCCCCGAGCCCGTGACCGTGT | | |
| | CTTGGAACTCCGGCGCTCTGACATCTGGCGTGCACA | | |
| | CCTTCCCTGCTGTGCTGCAGTCCTCTGGACTGTACT | | |
| | CCCTGTCCAGCGTGGTGACCGTGCCTTCTTCTAGCC | | |
| | TCGGCACCAAAACCTACACCTGCAACGTGGACCAC | | |
| | AAGCCTTCCAACACCAAAGTGGATAAGCGGGTGGA | | |
| | ATCTAAGTACGGCCCCCCCTGCCCTCCTTGTCCTGC | | |
| | TCCTGAGTTCCTGGGAGGACCTTCCGTGTTTCTGTTT | | |
| | CCTCCTAAGCCCAAGGACACCCTGATGATCTCTCGG | | |
| | ACCCCTGAAGTGACCTGTGTGGTCGTGGACGTGTCC | | |
| | CAAGAGGACCCTGAGGTGCAGTTCAACTGGTACGT | | |
| | GGATGGCGTGGAAGTGCACAACGCCAAGACCAAGC | | |
| | CTAGAGAAGAGCAGTTCAACTCTACCTACAGAGTG | LC | 140 |
| | GTGTCCGTGCTGACCGTGCTGCACCAGGACTGGCTG | | |
| | AACGGCAAAGAGTACAAGTGCAAGGTGTCTAACAA | | |
| | GGGCCTGCCTTCCTCCATCGAGAAGACCATCTCCAA | | |
| | GGCCAAGGGCCAACCCAGAGAGCCTCAGGTGTACA | | |
| | CACTGCCTCCAAGCCAAGAGGAAATGACCAAGAAC | | |
| | CAGGTGTCCCTGACCTGCCTGGTCAAGGGCTTCTAC | | |
| | CCTAGCGACATCGCCGTGGAATGGGAGTCCAATGG | | |
| | CCAGCCTGAGAACAACTACAAGACCACCCCACCTG | | |
| | TGCTGGACTCCGACGGCTCCTTCTTCCTGTACTCTA | | |
| | GACTGACAGTGGACAAGTCCAGATGGCAGGAGGGC | | |
| | AACGTGTTCTCCTGTTCTGTGATGCACGAGGCCCTG | | |
| | CACAACCACTACACCCAGAAGTCTCTGTCTCTGTCT | | |
| | CTGGGCAAGTGA | | |
| | ATGGATACTCGAGCACCAACTCAACTACTAGGACT | | |
| | GCTGCTCCTGTGGCTGCCTGGCGCTAGATGCGCCGA | | |
| | CATCCACATGACCCAGTCCCCATCCAGCCTGTCTGC | | |
| | TTCCGTGGGCGACACCGTCACAATCACCTGTCAGGC | | |
| | CTCTCAGAACATCAACTCCTACCTGTCCTGGTATCA | | |
| | GCAGAAACCCGGCAAGGCCCCTAAGCTGCTGATCT | | |
| | CCCTGGCCTCCGACCTGGCTTCTGGCGTGCCTTCTC | | |
| | GGTTCTCCGGCTCCGAGTCCGGCACCGACTACACCC | | |
| | TGACCATCAGCTCTCTGCAGCCTGAGGATTTTGCTA | | |
| | CCTACTACTGCCAGGGCTACTTCGCCGATTCTGGCT | | |
| | CTAGCGGCAGATCTTGGGCCTTCGGCCAAGGAACA | | |
| | AAGGTGGAAATCAAGAGAACCGTGGCCGCTCCTTC | | |
| | TGTGTTCATCTTCCCTCCTTCCGACGAGCAGCTGAA | | |
| | GTCCGGCACCGCTTCCGTGGTGTGCCTGCTGAACAA | | |
| | CTTCTACCCTAGAGAGGCCAAGGTGCAGTGGAAGG | | |
| | TGGACAACGCCCTGCAGTCTGGCAACTCCCAAGAG | | |
| | TCCGTCACCGAGCAGGACTCCAAGGACTCTACCTAC | | |
| | TCCCTGTCCTCCACCCTGACACTGAGCAAGGCCGAC | | |
| | TACGAGAAGCACAAGGTGTACGCCTGCGAGGTGAC | | |
| | CCACCAGGGCCTGTCTTCTCCTGTGACCAAGTCCTT | | |
| | CAACAGAGGCGAGTGCTGA | | |
| ROIS-4 | CAGGTGTCTCTCGAGGAGTCCGGCGGCGACCTGGT | HCVR | 141 |
| | CCAACCAGGCGGATCTCTGCGGCTGGCCTGCGTGG | | |
| | CTAGCGGCTTTTCCTTCTCCTCTGGCTGTTGGGTGTG | | |
| | CTGGGTGCGGCAGGCTCCTGGAAAAGGCCTGGAAT | | |
| | GGGTCTCCTGCATCTACACCGGCGATGGCGATACCT | | |
| | ACTACGCCAACTCTGTGAGAGGCAGATTCACCGTGT | | |
| | CTCGCAAGAACTCCACCACCTCCGTGTACCTGCAGA | | |
| | TGAACTCCCTGAGAGCCGACGACACCGCTCTGTACT | | |
| | TCTGCGCCTCTCCTCAGCCTGGCGACATCACATATC | | |
| | TGGGCTTCTGGGGCCCCGGAACACTGGTGACCGTGT | | |
| | CCAGC | | |
| | GACATCCACATGACCCAGTCCCCATCCAGCCTGTCT | LCVR | 142 |
| | GCTTCCGTGGGCGACACCGTCACAATCACCTGTCAG | | |
| | GCCTCTCAGAACATCAACTCCTACCTGTCCTGGTAT | | |
| | CAGCAGAAACCCGGCAAGGCCCCTAAGCTGCTGAT | | |
| | CTCCCTGGCCTCCGACCTGGCTTCTGGCGTGCCTTC | | |
| | TCGGTTCTCCGGCTCCGAGTCCGGCACCGACTACAC | | |
| | CCTGACCATCAGCTCTCTGCAGCCTGAGGATTTTGC | | |
| | TACCTACTACTGCCAGGGCTACTTCGCCGATTCTGG | | |
| | CTCTAGCGGCAGATCTTGGGCCTTCGGCCAAGGAA | | |
| | CAAAGGTGGAAATCAAGAGAACCGTG | | |
| | TCTGGCTGTTGGGTGTGC | HCDR1 | 143 |
| | TGCATCTACACCGGCGATGGCGATACCTACTACGCC | HCDR2 | 144 |
| | AACTCTGTGAGAGGC | | |
| | CCTCAGCCTGGCGACATCACATATCTGGGCTTC | HCDR3 | 145 |
| | CAGGCCTCTCAGAACATCAACTCCTACCTGTCC | LCDR1 | 146 |
| | CTGGCCTCCGACCTGGCTTCT | LCDR2 | 147 |
| | CAGGGCTACTTCGCCGATTCTGGCTCTAGCGGCAGA | LCDR3 | 148 |
| | TCTTGGGCC | | |
| | ATGGAAACTGGTCTACGATGGCTACTACTAGTGGCC | HC | 149 |
| | GTGCTGAAGGGCGTGCAGCAGGTGTCTCTCGAGGA | | |
| | GTCCGGCGGCGACCTGGTCCAACCAGGCGGATCTC | | |
| | TGCGGCTGGCCTGCGTGGCTAGCGGCTTTTCCTTCT | | |
| | CCTCTGGCTGTTGGGTGTGCTGGGTGCGGCAGGCTC | | |
| | CTGGAAAAGGCCTGGAATGGGTCTCCTGCATCTAC | | |
| | ACCGGCGATGGCGATACCTACTACGCCAACTCTGTG | | |
| | AGAGGCAGATTCACCGTGTCTCGCAAGAACTCCAC | | |
| | CACCTCCGTGTACCTGCAGATGAACTCCCTGAGAGC | | |
| | CGACGACACCGCTCTGTACTTCTGCGCCTCTCCTCA | | |
| | GCCTGGCGACATCACATATCTGGGCTTCTGGGGCCC | | |
| | CGGAACACTGGTGACCGTGTCCAGCGCCTCTACCA | | |
| | AGGGCCCTTCTGTGTTCCCTCTGGCCCCTTGCTCCC | | |
| | GGTCCACCTCCGAGTCTACAGCTGCTCTGGGCTGCC | | |
| | TGGTGAAGGACTACTTCCCCGAGCCCGTGACCGTGT | | |
| | CTTGGAACTCCGGCGCTCTGACATCTGGCGTGCACA | | |
| | CCTTCCCTGCTGTGCTGCAGTCCTCTGGACTGTACT | | |
| | CCCTGTCCAGCGTGGTGACCGTGCCTTCTTCTAGCC | | |
| | TCGGCACCAAAACCTACACCTGCAACGTGGACCAC | | |
| | AAGCCTTCCAACACCAAAGTGGATAAGCGGGTGGA | | |
| | ATCTAAGTACGGCCCCCCCTGCCCTCCTTGTCCTGC | | |
| | TCCTGAGTTCCTGGGAGGACCTTCCGTGTTTCTGTTT | | |
| | CCTCCTAAGCCCAAGGACACCCTGATGATCTCTCGG | | |
| | ACCCCTGAAGTGACCTGTGTGGTCGTGGACGTGTCC | | |
| | CAAGAGGACCCTGAGGTGCAGTTCAACTGGTACGT | | |
| | GGATGGCGTGGAAGTGCACAACGCCAAGACCAAGC | | |
| | CTAGAGAAGAGCAGTTCAACTCTACCTACAGAGTG | | |
| | GTGTCCGTGCTGACCGTGCTGCACCAGGACTGGCTG | | |
| | AACGGCAAAGAGTACAAGTGCAAGGTGTCTAACAA | LC | 150 |
| | GGGCCTGCCTTCCTCCATCGAGAAGACCATCTCCAA | | |
| | GGCCAAGGGCCAACCCAGAGAGCCTCAGGTGTACA | | |
| | CACTGCCTCCAAGCCAAGAGGAAATGACCAAGAAC | | |
| | CAGGTGTCCCTGACCTGCCTGGTCAAGGGCTTCTAC | | |
| | CCTAGCGACATCGCCGTGGAATGGGAGTCCAATGG | | |
| | CCAGCCTGAGAACAACTACAAGACCACCCCACCTG | | |
| | TGCTGGACTCCGACGGCTCCTTCTTCCTGTACTCTA | | |
| | GACTGACAGTGGACAAGTCCAGATGGCAGGAGGGC | | |
| | AACGTGTTCTCCTGTTCTGTGATGCACGAGGCCCTG | | |
| | CACAACCACTACACCCAGAAGTCTCTGTCTCTGTCT | | |
| | CTGGGCAAGTGA | | |
| | ATGGATACTCGAGCACCAACTCAACTACTAGGACT | | |
| | GCTGCTCCTGTGGCTGCCTGGCGCTAGATGCGCCGA | | |
| | CATCCACATGACCCAGTCCCCATCCAGCCTGTCTGC | | |
| | TTCCGTGGGCGACACCGTCACAATCACCTGTCAGGC | | |
| | CTCTCAGAACATCAACTCCTACCTGTCCTGGTATCA | | |
| | GCAGAAACCCGGCAAGGCCCCTAAGCTGCTGATCT | | |
| | CCCTGGCCTCCGACCTGGCTTCTGGCGTGCCTTCTC | | |
| | GGTTCTCCGGCTCCGAGTCCGGCACCGACTACACCC | | |
| | TGACCATCAGCTCTCTGCAGCCTGAGGATTTTGCTA | | |
| | CCTACTACTGCCAGGGCTACTTCGCCGATTCTGGCT | | |
| | CTAGCGGCAGATCTTGGGCCTTCGGCCAAGGAACA | | |
| | AAGGTGGAAATCAAGAGAACCGTGGCCGCTCCTTC | | |
| | TGTGTTCATCTTCCCTCCTTCCGACGAGCAGCTGAA | | |
| | GTCCGGCACCGCTTCCGTGGTGTGCCTGCTGAACAA | | |
| | CTTCTACCCTAGAGAGGCCAAGGTGCAGTGGAAGG | | |
| | TGGACAACGCCCTGCAGTCTGGCAACTCCCAAGAG | | |
| | TCCGTCACCGAGCAGGACTCCAAGGACTCTACCTAC | | |
| | TCCCTGTCCTCCACCCTGACACTGAGCAAGGCCGAC | | |
| | TACGAGAAGCACAAGGTGTACGCCTGCGAGGTGAC | | |
| | CCACCAGGGCCTGTCTTCTCCTGTGACCAAGTCCTT | | |
| | CAACAGAGGCGAGTGCTGA | | |
| ROIS-5 | CAGGTGTCTCTGGAAGAGTCCGGCGGCGATCTCGT | HCVR | 151 |
| | GCAACCAGGTGGAAGCCTGCGGCTGGCCTGCGTGG | | |
| | CTTCTGGATTTTCCTTCTCCTCTGGCTGTTGGGTGTG | | |
| | CTGGGTGCGGCAGGCTCCTGGCAAGGGCCTGGAGT | | |
| | GGGTCTCTTGCATCTACACCGGCGATGGCGACACCT | | |
| | ACTACGCCAACTCTGTCAGAGGCAGATTCACCGTGT | | |
| | CCAGACGGAACTCCACCACCTCCGTGTACCTGCAG | | |
| | ATGAACTCCCTGAGAGCCGACGACACAGCTCTGTA | | |
| | CTTCTGCGCCTCTCCTCAGCCTGGCGACATCACCTA | | |
| | TCTGGGCTTCTGGGGCCCCGGCACCCTGGTGACAGT | | |
| | GTCCAGC | | |
| | GACATCCACATGACCCAGTCCCCATCCAGCCTGTCT | LCVR | 152 |
| | GCTTCCGTGGGCGACACCGTCACAATCACCTGTCAG | | |
| | GCCTCTCAGAACATCAACTCCTACCTGTCCTGGTAT | | |
| | CAGCAGAAACCCGGCAAGGCCCCTAAGCTGCTGAT | | |
| | CTCCCTGGCCTCCGACCTGGCTTCTGGCGTGCCTTC | | |
| | TCGGTTCTCCGGCTCCGAGTCCGGCACCGACTACAC | | |
| | CCTGACCATCAGCTCTCTGCAGCCTGAGGATTTTGC | | |
| | TACCTACTACTGCCAGGGCTACTTCGCCGATTCTGG | | |
| | CTCTAGCGGCAGATCTTGGGCCTTCGGCCAAGGAA | | |
| | CAAAGGTGGAAATCAAGAGAACCGTG | | |
| | TCTGGCTGTTGGGTGTGC | HCDR1 | 153 |
| | TGCATCTACACCGGCGATGGCGACACCTACTACGCC | HCDR2 | 154 |
| | AACTCTGTCAGAGGC | | |
| | CCTCAGCCTGGCGACATCACCTATCTGGGCTTC | HCDR3 | 155 |
| | CAGGCCTCTCAGAACATCAACTCCTACCTGTCC | LCDR1 | 156 |
| | CTGGCCTCCGACCTGGCTTCT | LCDR2 | 157 |
| | CAGGGCTACTTCGCCGATTCTGGCTCTAGCGGCAGA | LCDR3 | 158 |
| | TCTTGGGCC | | |
| | ATGGAAACTGGTCTACGATGGCTACTACTAGTGGCC | HC | 159 |
| | GTGCTGAAGGGCGTGCAGCAGGTGTCTCTGGAAGA | | |
| | GTCCGGCGGCGATCTCGTGCAACCAGGTGGAAGCC | | |
| | TGCGGCTGGCCTGCGTGGCTTCTGGATTTTCCTTCTC | | |
| | CTCTGGCTGTTGGGTGTGCTGGGTGCGGCAGGCTCC | | |
| | TGGCAAGGGCCTGGAGTGGGTCTCTTGCATCTACAC | | |
| | CGGCGATGGCGACACCTACTACGCCAACTCTGTCA | | |
| | GAGGCAGATTCACCGTGTCCAGACGGAACTCCACC | | |
| | ACCTCCGTGTACCTGCAGATGAACTCCCTGAGAGCC | | |
| | GACGACACAGCTCTGTACTTCTGCGCCTCTCCTCAG | | |
| | CCTGGCGACATCACCTATCTGGGCTTCTGGGGCCCC | | |
| | GGCACCCTGGTGACAGTGTCCAGCGCCTCTACCAA | | |
| | GGGCCCTTCTGTGTTCCCTCTGGCCCCTTGCTCCCG | | |
| | GTCCACCTCCGAGTCTACAGCTGCTCTGGGCTGCCT | | |
| | GGTGAAGGACTACTTCCCCGAGCCCGTGACCGTGTC | | |
| | TTGGAACTCCGGCGCTCTGACATCTGGCGTGCACAC | | |
| | CTTCCCTGCTGTGCTGCAGTCCTCTGGACTGTACTC | | |
| | CCTGTCCAGCGTGGTGACCGTGCCTTCTTCTAGCCT | | |
| | CGGCACCAAAACCTACACCTGCAACGTGGACCACA | | |
| | AGCCTTCCAACACCAAAGTGGATAAGCGGGTGGAA | | |
| | TCTAAGTACGGCCCCCCCTGCCCTCCTTGTCCTGCT | | |
| | CCTGAGTTCCTGGGAGGACCTTCCGTGTTTCTGTTT | | |
| | CCTCCTAAGCCCAAGGACACCCTGATGATCTCTCGG | | |
| | ACCCCTGAAGTGACCTGTGTGGTCGTGGACGTGTCC | | |
| | CAAGAGGACCCTGAGGTGCAGTTCAACTGGTACGT | | |
| | GGATGGCGTGGAAGTGCACAACGCCAAGACCAAGC | | |
| | CTAGAGAAGAGCAGTTCAACTCTACCTACAGAGTG | | |
| | GTGTCCGTGCTGACCGTGCTGCACCAGGACTGGCTG | | |
| | AACGGCAAAGAGTACAAGTGCAAGGTGTCTAACAA | | |
| | GGGCCTGCCTTCCTCCATCGAGAAGACCATCTCCAA | | |
| | GGCCAAGGGCCAACCCAGAGAGCCTCAGGTGTACA | | |
| | CACTGCCTCCAAGCCAAGAGGAAATGACCAAGAAC | | |
| | CAGGTGTCCCTGACCTGCCTGGTCAAGGGCTTCTAC | | |
| | CCTAGCGACATCGCCGTGGAATGGGAGTCCAATGG | | |
| | CCAGCCTGAGAACAACTACAAGACCACCCCACCTG | | |
| | TGCTGGACTCCGACGGCTCCTTCTTCCTGTACTCTA | | |
| | GACTGACAGTGGACAAGTCCAGATGGCAGGAGGGC | | |
| | AACGTGTTCTCCTGTTCTGTGATGCACGAGGCCCTG | | |
| | CACAACCACTACACCCAGAAGTCTCTGTCTCTGTCT | | |
| | CTGGGCAAGTGA | | |
| | ATGGATACTCGAGCACCAACTCAACTACTAGGACT | LC 160 | |
| | GCTGCTCCTGTGGCTGCCTGGCGCTAGATGCGCCGA | | |
| | CATCCACATGACCCAGTCCCCATCCAGCCTGTCTGC | | |
| | TTCCGTGGGCGACACCGTCACAATCACCTGTCAGGC | | |
| | CTCTCAGAACATCAACTCCTACCTGTCCTGGTATCA | | |
| | GCAGAAACCCGGCAAGGCCCCTAAGCTGCTGATCT | | |
| | CCCTGGCCTCCGACCTGGCTTCTGGCGTGCCTTCTC | | |
| | GGTTCTCCGGCTCCGAGTCCGGCACCGACTACACCC | | |
| | TGACCATCAGCTCTCTGCAGCCTGAGGATTTTGCTA | | |
| | CCTACTACTGCCAGGGCTACTTCGCCGATTCTGGCT | | |
| | CTAGCGGCAGATCTTGGGCCTTCGGCCAAGGAACA | | |
| | AAGGTGGAAATCAAGAGAACCGTGGCCGCTCCTTC | | |
| | TGTGTTCATCTTCCCTCCTTCCGACGAGCAGCTGAA | | |
| | GTCCGGCACCGCTTCCGTGGTGTGCCTGCTGAACAA | | |
| | CTTCTACCCTAGAGAGGCCAAGGTGCAGTGGAAGG | | |
| | TGGACAACGCCCTGCAGTCTGGCAACTCCCAAGAG | | |
| | TCCGTCACCGAGCAGGACTCCAAGGACTCTACCTAC | | |
| | TCCCTGTCCTCCACCCTGACACTGAGCAAGGCCGAC | | |
| | TACGAGAAGCACAAGGTGTACGCCTGCGAGGTGAC | | |
| | CCACCAGGGCCTGTCTTCTCCTGTGACCAAGTCCTT | | |
| | CAACAGAGGCGAGTGCTGA | | |

Note:
The variant regions (HCVRs and LCVRs) and complementarity-determining regions (CDRs) were determined by ab Ysis (Swindells MB, et al. J Mol Biol. 2017 Feb. 3; 429(3): 356-364; http://www.abysis.org). CDRs can be identified using any of the numbering systems (e.g., Kabat, Chothia, Martin (enhanced Chothia), IMGT, AHo). The above representative CDRs were identified using the Kabat numbering system.

In some embodiments, the antibody or antigen-binding fragment thereof can be detectably labeled or conjugated to a toxin, a therapeutic agent, a polymer (e.g., polyethylene glycol (PEG)), a receptor, an enzyme, or a receptor ligand. For example, an antibody of the present invention may be coupled to a toxin (e.g., a tetanus toxin).

In another example, an antibody or antigen-binding fragment thereof disclosed herein may be coupled to a detectable tag. Such antibodies may be used within diagnostic assays to determine the presence of a S100A8/S100A8 homodimer, a S100A9/S100A9 homodimer, or a S100A8/S100A9 heterodimer. Examples of detectable tags include fluorescent proteins (i.e., green fluorescent protein, red fluorescent protein, yellow fluorescent protein), fluorescent markers (i.e., fluorescein isothiocyanate, rhodamine, texas red), radiolabels (i.e., 3H, 32P, 1251), enzymes (i.e., β-galactosidase, horseradish peroxidase, β-glucuronidase, alkaline phosphatase), or an affinity tag (i.e., avidin, biotin, streptavidin). Methods to couple antibodies to a detectable tag are known in the art. Harlow et al., Antibodies: A Laboratory Manual, page 319 (Cold Spring Harbor Pub. 1988).

### Fragment

In some embodiments, an antibody provided herein is an antibody fragment. Antibody fragments include, but are not limited to, Fab, Fab′, Fab′-SH, F(ab′)2, Fv, and single-chain Fv (scFv) fragments, and other fragments described below, e.g., diabodies, triabodies, tetrabodies, and single-domain antibodies. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (DOMANTIS, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### Chimeric and Humanized Antibodies

In some embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In some embodiments, an antibody provided herein is a human antibody. Human antibodies can be produced using various techniques known in the art or using techniques described herein. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For a review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352:624-628 (1991); Marks et al., J. Mol. Biol. 222:581-597 (1992); Marks and Bradbury, in Methods in Molecular Biology 248:161-175 (Lo, ed., Human Press, Totowa, N.J., 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12:725-734 (1993). Finally, naive libraries can also be made synthetically by cloning unrearranged V-gene segments from stem cells and using PCR primers containing random sequences to encode the highly variable CDR3 regions and to accomplish rearrangement in vitro, as described by Hoogenboom and Winter, J. Mol. Biol., 227:381-388 (1992). Patent publications describing human antibody phage libraries include, for example, U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

### Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

### Substitution, Insertion, and Deletion Variants

In some embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved antibody-dependent cell-mediated cytotoxicity (ADCC) and complement-dependent cytotoxicity (CDC).

Accordingly, an antibody of the invention can comprise one or more conservative modifications of the CDRs, heavy chain variable region, or light variable regions described herein. A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It substantially retains the activity of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent. Accordingly, within the scope of this disclosure are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., (XBLAST and NBLAST) can be used. (See www.ncbi.nlm.nih.gov).

As used herein, the term “conservative modifications” refers to amino acid modifications that do not significantly affect or alter the binding characteristics of the antibody containing the amino acid sequence. Such conservative modifications include amino acid substitutions, additions, and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: (i) amino acids with basic side chains (e.g., lysine, arginine, histidine), (ii) acidic side chains (e.g., aspartic acid, glutamic acid), (iii) uncharged polar side chains (e.g., glycine, asparagine, glutamine, serine, threonine, tyrosine, cysteine, tryptophan), (iv) nonpolar side chains (e.g., alanine, valine, leucine, isoleucine, proline, phenylalanine, methionine), (v) beta-branched side chains (e.g., threonine, valine, isoleucine), and (vi) aromatic side chains (e.g., tyrosine, phenylalanine, tryptophan, histidine).

Non-conservative substitutions will entail exchanging a member of one of these classes for another class.

An exemplary substitutional variant is an affinity matured antibody, which may be conveniently generated, e.g., using phage display-based affinity maturation techniques such as those described in, e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites are created or removed.

For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176, 195 by Hanai et al. describes a cell line with a functionally disrupted FUT8 gene, which encodes a fucosyltransferase, such that antibodies expressed in such a cell line exhibit hypofucosylation. PCT Publication WO 03/035835 by Presta describes a variant Chinese Hamster Ovary cell line, Led 3 cells, with reduced ability to attach fucose to Asn (297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyltransferases (e.g., beta(1,4)-N-acetylglucosaminyltransferase III (GnTIII)) such that antibodies expressed in the engineered cell lines exhibit increased bisecting GlcNac structures which result in increased ADCC activity of the antibodies (see also Umana et al. (1999) Nat. Biotech. 17:176-180).

### Fc Region Variants

The variable regions of the antibody described herein can be linked (e.g., covalently linked or fused) to an Fc, e.g., an IgG1, IgG2, IgG3 or IgG4 Fc, which may be of any allotype or isoallotype, e.g., for IgGl: Glm, Glml(a), Glm2(x), Glm3(f), Glml7(z); for IgG2: G2m, G2m23(n); for IgG3: G3m, G3m21(gl), G3m28(g5), G3 ml l(b0), G3m5(bl), G3ml3(b3), G3ml4(b4), G3m10(b5), G3ml5(s), G3ml6(t), G3m6(c3), G3m24(c5), G3m26(u), G3m27(v); and for K: Km, Kml, Km2, Km3 (see, e.g., Jefferies et al. (2009) mAbs 1:1). In some embodiments, the antibodies variable regions described herein are linked to an Fc that binds to one or more activating Fc receptors (FcγI, FcγIIa, or FcγIIIa), and thereby stimulate ADCC and may cause T cell depletion. In some embodiments, the antibody variable regions described herein are linked to an Fc that causes depletion.

In some embodiments, the antibody variable regions described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant, or derivative of the constant region. Suitable immunoglobulins include IgGI, IgG2, IgG3, IgG4, and other classes such as IgA, IgD, IgE, and IgM. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination. In some embodiments, an antibody of this invention has an Fc region other than that of a wild type IgA1. The antibody can have an Fc region from that of IgG (e.g., IgG1, IgG2, IgG3, and IgG4) or other classes such as IgA2, IgD, IgE, and IgM. The Fc can be a mutant form of IgA1.

The constant region of an immunoglobulin is responsible for many important antibody functions, including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgGl, IgG2, IgG3, and IgG4.

Ig molecules interact with multiple classes of cellular receptors. For example, IgG molecules interact with three classes of Fcγ receptors (FcγR) specific for the IgG class of antibodies, namely FcγRI, FcγRII, and FcγRIIL. The important sequences for the binding of IgG to the FcγR receptors have been reported to be located in the CH2 and CH3 domains. The serum half-life of an antibody is influenced by the ability of that antibody to bind to an FcR.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased CDC, (c) has increased or decreased affinity for Clq and/or (d) has increased or decreased affinity for an Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc., substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli, such as proline iminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the Clq binding site, may be removed from the Fc region. For example, one may delete or substitute the EKK sequence of human IgGl. In some embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgGl. Specific examples of variant Fc domains are disclosed, for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260.

In another example, one or more amino acids selected from amino acid residues 329, 331, and 322 can be replaced with a different amino acid residue such that the antibody has altered Clq binding and/or reduced or abolished CDC. This approach is described in further detail in U.S. Pat. Nos. 6,194,551.

In another example, one or more amino acid residues within amino acid positions 231 and 239 are altered to thereby alter the ability of the antibody to fix complement. This approach is described further in PCT Publication WO 94/29351.

In yet another example, the Fc region may be modified to increase ADCC and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 305I, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in abat (WO00/42072).

Other Fc modifications that can be made to Fcs are those for reducing or ablating binding to FcγR and/or complement proteins, thereby reducing or ablating Fc-mediated effector functions such as ADCC, antibody-dependent cellular phagocytosis (ADCP), and CDC. Exemplary modifications include but are not limited to substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; WO00/42072; WO01/58957; WO02/06919; WO04/016750; WO04/029207; WO04/035752; WO04/074455; WO04/099249; WO04/063351; WO05/070963; WO05/040217, WO05/092925 and WO06/020114).

Fc variants that enhance affinity for an inhibitory receptor FcγRIIb may also be used. Such variants may provide an Fc fusion protein with immune-modulatory activities related to FcγRIIb cells, including, for example, B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical- or immunological-based assays) known in the art, including but not limited to equilibrium methods (e.g., ELISA, or radioimmunoassay), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods, including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In some embodiments, the antibody is modified to increase its biological half-life. Various approaches are possible. For example, this may be done by increasing the binding affinity of the Fc region for FcRn. For example, one or more of the following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including, for example, 259I, 308F, 428L, 428M, 434S, 434H, 434F, 434Y, and 434M. Other variants that increase Fc binding to FcRn include: 250E, 250Q, 428L, 428F, 250Q/428L (Hinton et al, 2004, J. Biol. Chem. 279(8):6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 433I, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In some embodiments, hybrid IgG isotypes with particular biological characteristics may be used. For example, an IgGl/IgG3 hybrid variant may be constructed by substituting IgG 1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 422I, 435R, and 436F. In other embodiments described herein, an IgGl/IgG2 hybrid variant may be constructed by substituting IgG2 positions in the CH2 and/or CH3 region with amino acids from IgGl at positions where the two isotypes differ. Thus a hybrid variant IgG antibody may be constructed that comprises one or more substitutions, e.g., one or more of the following amino acid substitutions: 233E, 234L, 235L, 236G (referring to an insertion of a glycine at position 236), and 321H.

Moreover, the binding sites on human IgGl for FcγRI, FcγRII, FcγRIII, and FcRn have been mapped, and variants with improved binding have been described (see Shields, R. L. et al. (2001) J. Biol. Chem. 276:6591-6604). Specific mutations at positions 256, 290, 298, 333, 334, and 339 were shown to improve binding to FcγRIII. Additionally, the following combination mutants were shown to improve FcγRIII binding: T256A/S298A, S298A/E333A, S298A/K224A, and S298A/E333A/K334A, which has been shown to exhibit enhanced FcγRIIIa binding and ADCC activity (Shields et al., 2001). Other IgGl variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/1332E variant showed an enhanced capacity to deplete B cells in monkeys (Lazar et al., 2006). In addition, IgGl mutants containing L235V, F243L, R292P, Y300L, and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include S298A/E333A/L334A, S239D/I332E, S239D/I332E/A330L, L235V/F243L/R292P/Y300L/P396L, and M428L/N434S.

In some embodiments, an Fc is chosen that has reduced binding to FcγRs. An exemplary Fc, e.g., IgGl Fc, with reduced FcγR binding, comprises the following three amino acid substitutions: L234A, L235E, and G237A.

In some embodiments, an Fc is chosen that has reduced complement fixation. An exemplary Fc, e.g., IgGl Fc, with reduced complement fixation, has the following two amino acid substitutions: A330S and P331S.

In some embodiments, an Fc is chosen that has essentially no effector function, i.e., it has reduced binding to FcγRs and reduced complement fixation. An exemplary Fc, e.g., IgGl Fc, that is effectorless, comprises the following five mutations: L234A, L235E, G237A, A330S, and P331S.

When using an IgG4 constant domain, it is usually preferable to include the substitution S228P, which mimics the hinge sequence in IgG1 and thereby stabilizes IgG4 molecules.

### Multivalent Antibodies

In one embodiment, the antibodies of the invention may be monovalent or multivalent (e.g., bivalent, trivalent, etc.). As used herein, the term “valency” refers to the number of potential target binding sites associated with an antibody. Each target binding site specifically binds one target molecule or a specific position or locus on a target molecule. When an antibody is monovalent, each binding site of the molecule will specifically bind to a single antigen position or epitope. When an antibody comprises more than one target binding site (multivalent), each target binding site may specifically bind to the same or different molecules (e.g., may bind to different ligands or different antigens, or different epitopes or positions on the same antigen). See, for example, U.S.P.N. 2009/0130105. In each case, at least one of the binding sites will comprise an epitope, motif or domain associated with a DLL3 isoform.

In one embodiment, the antibodies are bispecific antibodies in which the two chains have different specificities, as described in Millstein et al., 1983, Nature, 305:537-539. Other embodiments include antibodies with additional specificities, such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As stated above, multivalent antibodies may immunospecifically bind to different epitopes of the desired target molecule or may immunospecifically bind to both the target molecule as well as a heterologous epitope, such as a heterologous polypeptide or solid support material. In some embodiments, the multivalent antibodies may include bispecific antibodies or trispecific antibodies. Bispecific antibodies also include cross-linked or “heteroconjugate” antibodies. For example, one of the antibodies in the heteroconjugate can be coupled to avidin, and the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art and are disclosed in U.S. Pat. No. 4,676,980, along with a number of cross-linking techniques.

In some embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

### Antibody Derivatives

An antibody provided herein may be further modified to contain additional nonproteinaceous moieties that are known in the art and readily available. The moieties suitable for derivatization of the antibody include but are not limited to water-soluble polymers.

Non-limiting examples of water-soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength and includes, but is not limited to, wavelengths that do not harm ordinary cells, but which heat the nonproteinaceous moiety to a temperature at which cells proximal to the antibody-nonproteinaceous moiety are killed.

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies described herein. See, for example, EP 0 154 316 by Nishimura et al. and EP0401384.

The present invention also encompasses a human monoclonal antibody described herein conjugated to a therapeutic agent, a polymer, a detectable label, or an enzyme. In one embodiment, the therapeutic agent is a cytotoxic agent. In one embodiment, the polymer is PEG.

### Nucleic Acids, Expression Cassettes, and Vectors

The present invention provides isolated nucleic acid segments that encode the polypeptides, peptide fragments, and coupled proteins of the invention. The nucleic acid segments also include segments that encode the same amino acids due to the degeneracy of the genetic code. For example, the amino acid threonine is encoded by ACU, ACC, ACA, and ACG and is therefore degenerate. It is intended that the invention includes all variations of the polynucleotide segments that encode for the same amino acids. Such mutations are known in the art (Watson et al., Molecular Biology of the Gene, Benjamin Cummings 1987). Mutations also include alteration of a nucleic acid segment to encode for conservative amino acid changes, for example, the substitution of leucine for isoleucine and so forth. Such mutations are also known in the art. Thus, the genes and nucleotide sequences of this disclosure include both naturally occurring sequences as well as mutant forms.

The nucleic acid segments of the invention may be contained within a vector. A vector may include, but is not limited to, any plasmid, phagemid, F-factor, virus, cosmid, or phage in a double- or single-stranded linear or circular form which may or may not be self-transmissible or mobilizable. The vector can also transform a prokaryotic or eukaryotic host either by integration into the cellular genome or exist extra-chromosomally (e.g., autonomous replicating plasmid with an origin of replication).

The nucleic acid segment in the vector can be under the control of, and operably linked to, an appropriate promoter or other regulatory elements for transcription in vitro or in a host cell, such as a eukaryotic cell, or a microbe, e.g., bacteria. The vector may be a shuttle vector that functions in multiple hosts. The vector may also be a cloning vector that typically contains one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech).

The nucleic acid segments of the invention may also be inserted into an expression vector. Typically, an expression vector contains prokaryotic DNA elements coding for a bacterial replication origin and an antibiotic resistance gene to provide for the amplification and selection of the expression vector in a bacterial host; regulatory elements that control initiation of transcription such as a promoter; and DNA elements that control the processing of transcripts such as introns, or a transcription termination/polyadenylation sequence.

Methods to introduce a nucleic acid segment into a vector are available in the art (Sambrook et al., Molecular Cloning: A Laboratory Manual, 3rd edition, Cold Spring Harbor Press, Cold Spring Harbor, N.Y. (2001)). Briefly, a vector into which a nucleic acid segment is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a nucleic acid segment into the vector. The nucleic acid segment that is to be inserted into the vector is treated with one or more restriction enzymes to create a linearized segment having a blunt end, a “sticky” end with a 5′ or a 3′ overhang, or any combination of the above. The nucleic acid segment may also be treated with a restriction enzyme and subsequently treated with another DNA modifying enzyme. Such DNA modifying enzymes include, but are not limited to, polymerase, exonuclease, phosphatase, or a kinase to create a nucleic acid segment that has characteristics useful for ligation of a nucleic acid segment into the vector.

The treated vector and nucleic acid segment are then ligated together to form a construct containing a nucleic acid segment according to methods available in the art (Sambrook et al., Molecular Cloning: A Laboratory Manual, 3rd edition, Cold Spring Harbor Press, Cold Spring Harbor, N.Y. (2001)). Briefly, the treated nucleic acid fragment, and the treated vector are combined in the presence of a suitable buffer and ligase. The mixture is then incubated under appropriate conditions to allow the ligase to ligate the nucleic acid fragment into the vector.

This disclosure also provides an expression cassette that contains a nucleic acid sequence capable of directing expression of a particular nucleic acid segment of the invention, either in vitro or in a host cell. Also, a nucleic acid segment may be inserted into the expression cassette such that an anti-sense message is produced. The expression cassette is an isolatable unit such that the expression cassette may be in linear form and functional for in vitro transcription and translation assays. The materials and procedures to conduct these assays are commercially available from Promega Corp. (Madison, Wis.). For example, an in vitro transcript may be produced by placing a nucleic acid sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a nucleic acid segment.

Such an expression cassette may contain one or a plurality of restriction sites allowing for placement of the nucleic acid segment under the regulation of a regulatory sequence. The expression cassette can also contain a termination signal operably linked to the nucleic acid segment as well as regulatory sequences required for proper translation of the nucleic acid segment. The expression cassette containing the nucleic acid segment may be chimeric, meaning that at least one of its components is heterologous with respect to at least one of its other components. The expression cassette may also be one that is naturally occurring but has been obtained in a recombinant form useful for heterologous expression. Expression of the nucleic acid segment in the expression cassette may be under the control of a constitutive promoter or an inducible promoter, which initiates transcription only when the host cell is exposed to some particular external stimulus.

The expression cassette may include in the 5′-3′ direction of transcription, a transcriptional and translational initiation region, a nucleic acid segment and a transcriptional and translational termination region functional in vivo and/or in vitro. The termination region may be native to the transcriptional initiation region, may be native to the nucleic acid segment, or may be derived from another source.

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences which may be a combination of synthetic and natural sequences. While regulatory sequences are not limited to promoters, some useful regulatory sequences include constitutive promoters, inducible promoters, regulated promoters, tissue-specific promoters, viral promoters, and synthetic promoters.

A promoter is a nucleotide sequence that controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors that control the effectiveness of transcription initiation in response to physiological or developmental conditions.

This disclosure also provides a construct containing a vector and an expression cassette. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a nucleic acid segment of the invention into a vector that itself contains regulatory sequences. Thus, an expression cassette is formed upon insertion of the nucleic acid segment into the vector. Vectors containing regulatory sequences are available commercially, and methods for their use are known in the art (Clonetech, Promega, Stratagene).

In another aspect, this disclosure also provides (i) a nucleic acid molecule encoding a polypeptide chain of the antibody or antigen-binding fragment thereof described herein; (ii) a vector comprising the nucleic acid molecule as described; and (iii) a cultured host cell comprising the vector as described. Also provided is a method for producing a polypeptide, comprising: (a) obtaining the cultured host cell as described; (b) culturing the cultured host cell in a medium under conditions permitting expression of a polypeptide encoded by the vector and assembling of an antibody or fragment thereof; and (c) purifying the antibody or fragment from the cultured cell or the medium of the cell.

### Methods of Production

Antibodies may be produced using recombinant methods and compositions, e.g., as described in U.S. Pat. No. 4,816,567. In one embodiment, an isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, a nucleic acid encoding an antibody, e.g., as described herein, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified, which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include CHO cells, including DHFR-CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Compositions and Kits

In another aspect, this disclosure provides a pharmaceutical composition comprising the antibodies or antigen-binding fragments described herein formulated together with a pharmaceutically acceptable carrier. The composition may optionally contain one or more additional pharmaceutically active ingredients, such as another antibody or a therapeutic agent.

In some embodiments, the pharmaceutical composition comprises two or more of the antibody or antigen-binding fragment thereof described herein, such as any combinations of the antibody or antigen-binding fragment thereof comprising a heavy chain and a light chain that comprise the respective amino acid sequences described herein.

In some embodiments, the pharmaceutical composition can also be administered in a combination therapy with, for example, another immune-stimulatory agent, an antiviral agent, a vaccine, etc. In some embodiments, a composition comprises an antibody of this invention at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

In some embodiments, the second therapeutic agent comprises an anti-inflammatory drug or an anti-cancer agent.

Also within the scope of this disclosure is use of the pharmaceutical composition in the preparation of a medicament for the diagnosis, prophylaxis, treatment, or combination thereof of a condition (e.g., a disease or disorder).

The pharmaceutical composition can comprise any number of excipients. Excipients that can be used include carriers, surface-active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

In some embodiments, the pharmaceutical composition can be used for intravenous, intramuscular, subcutaneous, parenteral, spinal, or epidermal administration (e.g., by injection or infusion). Depending on the route of administration, the active compound can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the present invention described herein can be administered via a non-parenteral route, such as a topical, epidermal, or mucosal route of administration, e.g., intranasally, orally, vaginally, rectally, sublingually, or topically.

In some embodiments, the pharmaceutical compositions can be prepared in many forms that include tablets, hard or soft gelatin capsules, aqueous solutions, suspensions, liposomes, and other slow-release formulations, such as shaped polymeric gels. An oral dosage form may be formulated such that the antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups, or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

An antibody can be formulated for parenteral administration (e.g., by injection, for example, bolus injection or continuous infusion) and may be presented in unit dosage form in ampules, prefilled syringes, small volume infusion containers, or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

For administration by inhalation, an antibody can be conveniently delivered from an insufflator, nebulizer, a pressurized pack, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, an antibody may take the form of a dry powder composition, for example, a powder mix of a modulator and a suitable powder base such as lactose or starch. The powder composition may be presented in a unit dosage form in, for example, capsules or cartridges or, e.g., gelatin or blister packs from which the powder may be administered with the aid of an inhalator or insufflator. For intra-nasal administration, an antibody may be administered via a liquid spray, such as via a plastic bottle atomizer.

Pharmaceutical compositions of the invention may also contain other ingredients such as flavorings, colorings, anti-microbial agents, or preservatives. It will be appreciated that the amount of an antibody required for use in treatment will vary not only with the particular carrier selected but also with the route of administration, the nature of the condition being treated, and the age and condition of the patient. Ultimately the attendant health care provider may determine a proper dosage. In addition, a pharmaceutical composition may be formulated as a single unit dosage form.

The pharmaceutical composition of the present invention can be in the form of sterile aqueous solutions or dispersions. It can also be formulated in a microemulsion, liposome, or other ordered structure suitable for high drug concentration.

An antibody of the present invention described herein can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

The amount of active ingredient that can be combined with a carrier material to produce a single dosage form will vary depending upon the subject being treated and the particular mode of administration and will generally be that amount of the composition, which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

Dosage regimens can be adjusted to provide the optimum desired response (e.g., a therapeutic response). For example, a single bolus can be administered, several divided doses can be administered over time, or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the antibody can be administered as a sustained release formulation, in which case less frequent administration is required. For administration of the antibody, the dosage ranges from about 0.0001 to 800 mg/kg, and more usually 0.01 to 5 mg/kg, of the host body weight. For example, dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/ml, and in some methods, about 25-300 μg/ml. A “therapeutically effective dosage” of an antibody of the invention preferably results in a decrease in severity of disease symptoms, an increase in frequency and duration of disease symptom-free periods, or a prevention of impairment or disability due to the disease affliction.

The pharmaceutical composition can be a controlled release formulation, including implants, transdermal patches, and microencapsulated delivery systems. Biodegradable, biocompatible polymers can be used, such as ethylene-vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In some embodiments, the human monoclonal antibodies of the invention described herein can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs. See, e.g., U.S. Pat. Nos. 4,522,811; 5,374,548; 5,416,016; and 5,399,331; V. V. Ranade (1989) Clin. Pharmacol. 29:685; Umezawa et al., (1988) Biochem. Biophys. Res. Commun. 153:1038; Bloeman et al. (1995) FEBS Lett. 357:140; M. Owais et al. (1995) Antimicrob. Agents Chemother. 39:180; Briscoe et al. (1995) Am. Physiol. 1233:134; Schreier et al. (1994). Biol. Chem. 269:9090; Keinanen and Laukkanen (1994) FEBS Lett. 346:123; and Killion and Fidler (1994) Immunomethods 4:273.

In some embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor-mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.), and may be administered together with other biologically active agents. Administration can be systemic or local. The pharmaceutical composition can also be delivered in a vesicle, in particular, a liposome (see, for example, Langer (1990) Science 249:1527-1533).

The use of nanoparticles to deliver the antibodies of the present invention is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389), incorporated herein by reference. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity to the composition's target, thus requiring only a fraction of the systemic dose.

The injectable preparations may include dosage forms for intravenous, subcutaneous, intracutaneous, intracranial, intraperitoneal, intramuscular injections, drip infusions, etc. These injectable preparations may be prepared by methods publicly known. For example, the injectable preparations may be prepared, e.g., by dissolving, suspending, or emulsifying the antibody or its salt described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pens and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to, the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.) and the HUMIRA™ Pen (Abbott Labs, Abbott Park, IL), to name only a few.

Advantageously, the pharmaceutical compositions for oral or parenteral use described herein are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 300 mg and in about 10 to about 300 mg for the other dosage forms.

In another aspect, this disclosure provides a kit comprising a pharmaceutically acceptable dose unit of the antibody or antigen-binding fragment thereof or the pharmaceutical composition as described herein. Also within the scope of this disclosure is a kit that comprises: the antibody or antigen-binding fragment thereof as described; and a least one detection reagent that binds specifically to the antibody or antigen-binding fragment thereof.

In some embodiments, the kit also includes a container that contains the composition and optionally informational material. The informational material can be descriptive, instructional, marketing, or other material that relates to the methods described herein and/or the use of the agents for therapeutic benefit. In an embodiment, the kit also includes an additional therapeutic agent, as described herein. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In one embodiment, the informational material relates to methods of administering the composition, e.g., in a suitable dose, dosage form, or mode of administration (e.g., dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, audio recording, or information that contains a link or address to substantive material.

The kit can include one or more containers for the composition. In some embodiments, the kit contains separate containers, dividers, or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading. Such a kit may optionally contain a syringe to allow for injection of the antibody contained within the kit into an animal, such as a human.

### Methods of Use

### Methods of Treatment

The antibodies, compositions, and formulations described herein can be used to treat a disease or disorder, such as leukemia, myelodysplastic syndromes (MDS), or an inflammatory and autoimmune disease in a subject in need thereof. In some embodiments, the method comprises administering to the subject the antibody or antigen-binding fragment thereof, the nucleic acid molecule, the vector, or the pharmaceutical composition, as described herein.

In some embodiments, the leukemia is acute lymphoblastic leukemia (ALL), acute myeloid leukemia (AML), chronic lymphocytic leukemia (CLL), chronic myeloid leukemia (CML), or chronic myelomonocytic leukemia (CMML).

In some embodiments, the inflammatory and autoimmune disease is non-neoplastic inflammation.

In some embodiments, the method further comprises administering to the subject an additional therapeutic agent or therapy. In some embodiments, the additional therapeutic agent or therapy comprises at least one of daunorubicin, doxorubicin, and cytarabine.

In some embodiments, the antibody or antigen-binding fragment thereof is administered before or after the additional therapeutic agent or therapy. In some embodiments, the antibody or antigen-binding fragment thereof is administered concurrently with the additional therapeutic agent or therapy.

In some embodiments, the antibody or antigen-binding fragment thereof or the additional therapeutic agent or therapy is administered to the subject intravenously, subcutaneously, or intraperitoneally.

In another aspect, this disclosure provides use of an antibody or antigen-binding fragment thereof for the manufacture of a medicament for the method described above.

In another aspect, this disclosure provides an antibody or antigen-binding fragment thereof for use in the method described above.

In another aspect, this disclosure further provides a method of stimulating cell differentiation or cell growth of a cell in a subject in need thereof. In some embodiments, the method comprises administering to the subject the antibody or antigen-binding fragment thereof, the nucleic acid molecule, the vector, or the pharmaceutical composition, as described herein.

In yet another aspect, this disclosure additionally provides a method of stimulating cell differentiation or cell growth of a cell ex vivo. In some embodiments, the method comprises contacting the cell with the antibody or antigen-binding fragment thereof, the nucleic acid molecule, the vector, or the pharmaceutical composition, as described herein.

In some embodiments, the cell comprises an acute myeloid leukemia cell or a red blood cell.

In some embodiments, the subject has leukemia or a myelodysplastic syndrome (MDS). In some embodiments, the leukemia is acute lymphoblastic leukemia (ALL), acute myeloid leukemia (AML), chronic lymphocytic leukemia (CLL), chronic myeloid leukemia (CML), or chronic myelomonocytic leukemia (CMML).

### Combination Therapies

Combination therapies may include an antibody or antigen-binding fragment as disclosed herein and any additional therapeutic agent that may be advantageously combined with an antibody as disclosed or with a biologically active fragment of an antibody of the invention. The antibodies may be combined synergistically with one or more drugs or therapies used to treat a disease or disorder. In some embodiments, the antibodies may be combined with a second therapeutic agent to ameliorate one or more symptoms of said disease. In some embodiments, the antibodies may be combined with a second antibody to provide synergistic activity in ameliorating one or more symptoms of said disease. In some embodiments, the first antibody or antigen-binding fragment thereof is administered before, after, or concurrently with the second antibody or antigen-binding fragment thereof.

In some embodiments, the second therapeutic agent is another antibody or antigen-binding fragment thereof targeting a S100A8/S100A8 homodimer, a S100A9/S100A9 homodimer, or a S100A8/S100A9 hetero-oligomer (such as a S100A8/S100A9 heterodimer). It is contemplated herein to use a combination (“cocktail”) of antibodies with broad neutralization or inhibitory activity against a S100A8/S100A8 homodimer, a S100A9/S100A9 homodimer, or a S100A8/S100A9 hetero-oligomer. In some embodiments, non-competing antibodies may be combined and administered to a subject in need thereof. In some embodiments, the antibodies comprising the combination bind to distinct non-overlapping epitopes on the protein. In some embodiments, the second antibody may possess a longer half-life in human serum.

As used herein, the term “in combination with” means that additional therapeutically active component(s) may be administered prior to, concurrent with, or after the administration of an antibody or antigen-binding fragment as disclosed herein. The term “in combination with” also includes sequential or concomitant administration of an antibody or antigen-binding fragment as disclosed herein and a second therapeutic agent.

The additional therapeutically active component(s) may be administered to a subject prior to administration of an antibody or antigen-binding fragment as disclosed herein. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an antibody or antigen-binding fragment as disclosed herein. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an antibody or antigen-binding fragment as disclosed herein. “Concurrent” administration, for purposes of this disclosure, includes, e.g., administration of an antibody or antigen-binding fragment as disclosed herein and an additional therapeutically active component to a subject in a single dosage form, or in separate dosage forms administered to the subject within about 30 minutes or less of each other. If administered in separate dosage forms, each dosage form may be administered via the same route (e.g., both the disclosed antibody or antigen-binding fragment and the additional therapeutically active component may be administered intravenously, etc.); alternatively, each dosage form may be administered via a different route (e.g., the disclosed antibody or antigen-binding fragment may be administered intravenously, and the additional therapeutically active component may be administered orally). In any event, administering the components in a single dosage form, in separate dosage forms by the same route, or in separate dosage forms by different routes are all considered “concurrent administration,” for purposes of the present disclosure. For purposes of the present disclosure, administration of the disclosed antibody or antigen-binding fragment “prior to,” “concurrent with,” or “after” (as those terms are defined hereinabove) administration of an additional therapeutically active component is considered administration of the disclosed antibody or antigen-binding fragment “in combination with” an additional therapeutically active component.

The present invention includes pharmaceutical compositions in which the disclosed antibody or antigen-binding fragment is co-formulated with one or more of the additional therapeutically active component(s) as described elsewhere herein.

### Administration Regimens

According to certain embodiments, a single dose of the disclosed antibody or antigen-binding fragment (or a pharmaceutical composition comprising a combination of the disclosed antibody or antigen-binding fragment and any of the additional therapeutically active agents mentioned herein) may be administered to a subject in need thereof. According to certain embodiments of the present invention, multiple doses of the disclosed antibody or antigen-binding fragment (or a pharmaceutical composition comprising a combination of the disclosed antibody or antigen-binding fragment and any of the additional therapeutically active agents mentioned herein) may be administered to a subject over a defined time course. The methods according to this aspect of the invention comprise sequentially administering to a subject multiple doses of the disclosed antibody or antigen-binding fragment. As used herein, “sequentially administering” means that each dose of the disclosed antibody or antigen-binding fragment is administered to the subject at a different point in time, e.g., on different days separated by a predetermined interval (e.g., hours, days, weeks, or months). The present invention includes methods that comprise sequentially administering to the patient a single initial dose of the disclosed antibody or antigen-binding fragment, followed by one or more secondary doses of the disclosed antibody or antigen-binding fragment, and optionally followed by one or more tertiary doses of the antibody or antigen-binding fragment.

The terms “initial dose,” “secondary doses,” and “tertiary doses,” refer to the temporal sequence of administration of the disclosed antibody or antigen-binding fragment. Thus, the “initial dose” is the dose, which is administered at the beginning of the treatment regimen (also referred to as the “baseline dose”); the “secondary doses” are the doses, which are administered after the initial dose; and the “tertiary doses” are the doses which are administered after the secondary doses. The initial, secondary, and tertiary doses may all contain the same amount of the antibody or antigen-binding fragment, but generally may differ from one another in terms of frequency of administration. In some embodiments, however, the amount of the antibody or antigen-binding fragment contained in the initial, secondary and/or tertiary doses varies from one another (e.g., adjusted up or down as appropriate) during the course of treatment. In some embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present invention, each secondary and/or tertiary dose is administered 1 to 48 hours (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) after the immediately preceding dose. The phrase “the immediately preceding dose,” as used herein, means, in a sequence of multiple administrations, the dose of the antibody or antigen-binding fragment, which is administered to a patient prior to the administration of the very next dose in the sequence with no intervening doses.

The methods, according to this aspect of the invention, may comprise administering to a patient any number of secondary and/or tertiary doses of the antibody or antigen-binding fragment. For example, in some embodiments, only a single secondary dose is administered to the patient. In other embodiments, two or more (e.g., 2, 3, 4, 5, 6, 7, 8, or more) secondary doses are administered to the patient. Likewise, in some embodiments, only a single tertiary dose is administered to the patient. In other embodiments, two or more (e.g., 2, 3, 4, 5, 6, 7, 8, or more) tertiary doses are administered to the patient.

In some embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

### Diagnostic Uses of the Antibodies

In another aspect, this disclosure provides a method for detecting the presence of a S100 protein in a sample. In some embodiments, the method comprises: (i) contacting the sample with the antibody or antigen-binding fragment thereof described above; and (ii) determining binding of the antibody or antigen-binding fragment to the alarmin protein, wherein binding of the antibody or antigen-binding fragment thereof to the alarmin protein is indicative of the presence of the S100 protein in the sample.

In some embodiments, the S100 protein comprise a S100A8/S100A8 homodimer, a S100A9/S100A9 homodimer, or a S100A8/S100A9 hetero-oligomer (such as a S100A8/S100A9 heterodimer).

In some embodiments, the antibody or antigen-binding fragment thereof is conjugated to a label. In some embodiments, the label is selected from a fluorescent label, a chemiluminescent label, a radiolabel, and an enzyme.

In some embodiments, the method comprises contacting a secondary antibody with the antibody or antigen-binding fragment thereof. In some embodiments, the step of determining comprises performing a competitive binding assay or ELISA.

In some embodiments, the sample comprises a blood sample. In some embodiments, the method comprises binding the sample to a solid support. In some embodiments, the solid support is selected from microparticles, microbeads, magnetic beads, and an affinity purification column. In some embodiments, the disclosed antibody or antigen-binding fragment is labeled with a detectable label or reporter molecule or used as a capture ligand to selectively isolate antibody or antigen-binding fragment from patient samples. Alternatively, an unlabeled antibody or antigen-binding fragment can be used in diagnostic applications in combination with a secondary antibody, which is itself detectably labeled. The detectable label or reporter molecule can be a radioisotope, such as H, C, P, S, or I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure the antibody or antigen-binding fragment in a sample include enzyme-linked immunosorbent assay (ELISA), radioimmunoassay (RIA), and fluorescence-activated cell sorting (FACS).

In some embodiments, the antibody or antigen-binding fragment thereof is conjugated to a label. In some embodiments, the step of detecting comprises contacting a secondary antibody with the antibody or antigen-binding fragment thereof and wherein the secondary antibody comprises a label. In some embodiments, the label includes a fluorescent label, a chemiluminescent label, a radiolabel, and an enzyme.

In some embodiments, the step of detecting comprises detecting fluorescence or chemiluminescence. In some embodiments, the step of detecting comprises a competitive binding assay or ELISA.

In some embodiments, the method further comprises binding the sample to a solid support. In some embodiments, the solid support includes microparticles, microbeads, magnetic beads, and an affinity purification column.

Samples that can be used in diagnostic assays according to the present disclosure include any tissue or fluid sample obtainable from a patient, which contains detectable quantities of S100 proteins or fragments thereof, such as a S100A8/S100A8 homodimer, a S100A9/S100A9 homodimer, or a S100A8/S100A9 hetero-oligomer (e.g., a S100A8/S100A9 heterodimer) under normal or pathological conditions. Generally, levels of S100 proteins in a particular sample obtained from a healthy patient (e.g., a patient not afflicted with a disease associated with S100 proteins) will be measured to initially establish a baseline or standard level of S100 proteins. This baseline level of S100 proteins can then be compared against the levels of S100 proteins measured in samples obtained from individuals suspected of having a S100 proteins-associated condition or symptoms associated with such condition.

The antibodies specific for S100 proteins may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing a N-terminal biotin will be oriented such that the C-terminal portion of the peptide will be distal to the surface.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The heavy chain variable region CDRs and FRs are HFR1, HCDR1, HFR2, HCDR2, HFR3, HCDR3, and HFR4. The light chain variable region CDRs and FRs are LFR1, LCDR1, LFR2, LCDR2, LFR3, LCDR3, and LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the VL, VH, CL, and CH1 domains; (ii) a F(ab′) 2 fragment, a bivalent fragment comprising two Fab fragments linked by a disulfide bridge at the hinge region; (iii) a Fab′ fragment, which is essentially a Fab with part of the hinge region (see, FUNDAMENTAL IMMUNOLOGY (Paul ed., 3rd ed. 1993)); (iv) a Fd fragment consisting of the VH and CH1 domains; (v) a Fv fragment consisting of the VL and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as intact antibodies.

An “isolated antibody,” as used herein, is intended to refer to an antibody that is substantially free of other antibodies having different antigenic specificities. An isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created, or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and

VL regions of the recombinant antibodies are sequences that, while derived from and related to human germline VH and VL sequences, may not naturally exist within the human antibody germline repertoire in vivo.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species, and the constant region sequences are derived from another species, such as an antibody in which the variable region sequences are derived from a mouse antibody, and the constant region sequences are derived from a human antibody. The term can also refer to an antibody in which its variable region sequence or CDR(s) is derived from one source (e.g., an IgAQ1 antibody), and the constant region sequence or Fc is derived from a different source (e.g., a different antibody, such as an IgG, IgA2, IgD, IgE or IgM antibody).

The invention encompasses isolated or substantially purified nucleic acids, peptides, polypeptides, or proteins. In the context of the present invention, an “isolated” nucleic acid, DNA or RNA molecule or an “isolated” polypeptide is a nucleic acid, DNA molecule, RNA molecule, or polypeptide that exists apart from its native environment and is therefore not a product of nature. An isolated nucleic acid, DNA molecule, RNA molecule, or polypeptide may exist in a purified form or may exist in a non-native environment such as, for example, a transgenic host cell. A “purified” nucleic acid molecule, peptide, polypeptide, or protein, or a fragment thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein, peptide, or polypeptide that is substantially free of cellular material includes preparations of protein, peptide, or polypeptide having less than about 30%, 20%, 10%, or 5% (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide, or protein. For example, a peptide or polypeptide fragment can have is at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids, or less than about 250 amino acids in length. Preferably the peptide fragment can elicit an immune response when used to inoculate an animal. A peptide fragment may be used to elicit an immune response by inoculating an animal with a peptide fragment in combination with an adjuvant, a peptide fragment that is coupled to an adjuvant, or a peptide fragment that is coupled to arsanilic acid, sulfanilic acid, an acetyl group, or a picryl group. A peptide fragment can include a non-amide bond and can be a peptidomimetic.

As used herein, the term “conjugate,” “conjugation,” or “linked” as used herein refers to the attachment of two or more entities to form one entity. A conjugate encompasses both peptide-small molecule conjugates as well as peptide-protein/peptide conjugates.

The term “recombinant,” as used herein, refers to antibodies or antigen-binding fragments thereof of the invention created, expressed, isolated, or obtained by technologies or methods known in the art as recombinant DNA technology, which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Example conservative amino acid substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of molecule X for its partner Y can generally be represented by the dissociation constant (KD). Affinity can be measured by common methods known in the art, including those described herein.

The term “specifically binds,” “binds specifically to,” or the like, refers to an antibody that binds to a single epitope, e.g., under physiologic conditions., but which does not bind to more than one epitope. Accordingly, an antibody that specifically binds to a polypeptide will bind to an epitope that is present on the polypeptide, but which is not present on other polypeptides. Specific binding can be characterized by an equilibrium dissociation constant of at least about 1×10⁻⁸ M or less (e.g., a smaller KD denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to an antigen, such as a S100A8/S100A8 homodimer, a S100A9/S100A9 homodimer, or a S100A8/S100A9 heterodimer.

In some embodiments, the antibody binds to an antigen with “high affinity,” namely with a KD of 1×10⁻⁷ M or less, more preferably 5×10⁻⁸ M or less, more preferably 3×10⁻⁸ M or less, more preferably 1×10⁻⁸ M or less, more preferably 5×10⁻⁹ M or less or even more preferably 1×10⁻⁹ M or less, as determined by surface plasmon resonance, e.g., BIACORE. The term “does not substantially bind” to a protein or cells, as used herein, means does not bind or does not bind with a high affinity to the protein or cells, i.e., binds to the protein or cells with a K_{D} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “Kassoc” or “Ka,” as used herein, is intended to refer to the association rate of a particular antibody-antigen interaction, whereas the term “Kdis” or “Kd,” as used herein, is intended to refer to the dissociation rate of a particular antibody-antigen interaction. The term “KD,” as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface plasmon resonance, preferably using a biosensor system such as a BIACORE system.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In some embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope, or adjacent epitopes (e.g., as evidenced by steric hindrance). Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “epitope,” as used herein, refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In some embodiments, epitopes may include determinants that are chemically active surface groupings of molecules such as amino acids, sugar side chains, phosphoryl groups, or sulfonyl groups, and, In some embodiments, may have specific three-dimensional structural characteristics, and/or specific charge characteristics. An epitope typically includes at least 3, 4, 5, 6, 7, 8, 9, 10, 11, 12, 13, 14, or 15 amino acids in a unique spatial conformation. Methods for determining what epitopes are bound by a given antibody (i.e., epitope mapping) are well known in the art and include, for example, immunoblotting and immune-precipitation assays, wherein overlapping or contiguous peptides from an antigen are tested for reactivity with a given antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “epitope mapping” refers to the process of identification of the molecular determinants for antibody-antigen recognition.

The term “binds to an epitope” or “recognizes an epitope” with reference to an antibody or antibody fragment refers to continuous or discontinuous segments of amino acids within an antigen. Those of skill in the art understand that the terms do not necessarily mean that the antibody or antibody fragment is in direct contact with every amino acid within an epitope sequence.

The term “binds to the same epitope” with reference to two or more antibodies means that the antibodies bind to the same, overlapping, or encompassing continuous or discontinuous segments of amino acids. Those of skill in the art understand that the phrase “binds to the same epitope” does not necessarily mean that the antibodies bind to or contact exactly the same amino acids. The precise amino acids that the antibodies contact can differ. For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

As used herein, the term “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell, or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

The term “detectable label” as used herein refers to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof that is capable of exhibiting fluorescence in the detectable range.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., neuronal disorders, autoimmune diseases, and cardiovascular diseases), and/or who has been diagnosed with inflammatory disorders. In some embodiments, the subject is a mammal. In some embodiments, the subject is human.

As used herein, the term “disease” is intended to be generally synonymous and is used interchangeably with the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

As used herein, the term “treating” or “treatment” of any disease or disorder refers in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically (e.g., stabilization of a discernible symptom), physiologically (e.g., stabilization of a physical parameter), or both. In yet another embodiment, “treating” or “treatment” refers to preventing or delaying the onset or development or progression of the disease or disorder.

The terms “prevent,” “preventing,” “prevention,” “prophylactic treatment” and the like refer to reducing the probability of developing a disorder or condition in a subject, who does not have, but is at risk of or susceptible to developing a disorder or condition.

The terms “decrease,” “reduced,” “reduction,” “decrease,” or “inhibit” are all used herein generally to mean a decrease by a statistically significant amount. However, for avoidance of doubt, “reduced,” “reduction,” “decrease,” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “therapeutic effect” is art-recognized and refers to a local or systemic effect in animals, particularly mammals, and more particularly humans, caused by a pharmacologically active substance.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, absorption delaying agents, and the like that are compatible with the activity of one or more components of this disclosure, and are physiologically acceptable to the subject. Supplementary active compounds may also be incorporated into the compositions.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest, such as antibodies. The sample may be any tissue sample from the subject. The sample may comprise protein from the subject.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition that is “substantially free” from Y may be completely free from Y. Where necessary, the word “substantially” may be omitted from the definition of the invention.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials, methods, and instrumentation used in EXAMPLE 2.

### Recombinant Human Proteins and IgG Controls

S100A8, S100A9, and S100A8/A9 were obtained from multiple companies and were identified by a suffix of the company name. S100A8-Sino (11138-H08B), S100A9-Sino (11145-H08B) and S100A8/A9-Sino (CT002-H0822B) were purchased from Sino Biological. S100A8/A9-R&D (8916-S8-050), S100A8/A9-kactus (CAL-HE101) and S100A8/A9-Arotec (ATC04-02) were obtained from R&D Systems, Kactus Biosystems, and Arotec Diagnostics, respectively. While CD69 (11150-H08H) was obtained from Sino Biological, the other receptors, TLR4/MD2 (3146-TM-050/CF)), RAGE-Fc (1145-RG-050), CD146-Fc (9709-MA-050), CD147-Fc (972-EMN-050), Siglec-9 (1139-SL-050) and CD33-Fc (1137-SL-050) were all purchased from R&D Systems. Naïve serum Rabbit IgG (AB-105-C) and mouse IgG (18765-5 MG) were purchased from R&D Systems and Sigma-Aldrich, respectively.

### Characterization of the Specificity of Antisera and Antibodies

S100A8, S100A9, and S100A8/A9 were coated onto Maxisorp ELISA plates at a concentration of 0.5 or 1 μg/ml overnight. The plates were incubated with a serial dilution of antiserum or antibody at a concentration of 0.2 μg/ml in Calcium-containing TBS buffer (7 mM Tris, 150 mM, 2 mM CaCl2, and 0.1% BSA). The bound rabbit antibody was probed with HRP-conjugated goat anti-rabbit IgG (#7074, Cell Signaling), whereas the bound mouse chimera antibody was detected by HRP-conjugated anti-mouse-IgG (#7076, Cell Signaling). The data are representative of at least two independent experiments.

Assessment of Inhibition of Binding of S100A8, S100A9 or S100A8/A9 to their Receptors by Rabbit or Mouse Chimera Antibodies

ELISA plate was coated with receptors at a concentration of 5 μg/ml overnight. Binding of S100A8, S100A9 or S100A8/A9 at a concentration of 1 μg/ml to receptors was performed in the presence or absence or rabbit or mouse chimera antibody (in this case, as a blocking antibody). The plate-bound S100A proteins were detected with appropriate primary and secondary antibodies. A decrease in signal in the presence of an antibody indicates a suppression of binding of s100A proteins to receptors by this antibody. For inhibition by rabbit antibodies, His-tagged S100A8-Sino, S100A9-Sino, and S100A8/A9-Kactus were used, followed by mouse anti-His antibody (MAB050-100, R&D Systems, 0.5 μg/ml) and HRP-conjugated anti-Mouse IgG. For inhibition of mouse chimera antibodies, both His-tagged and untagged S100A proteins were used, followed by rabbit antiserum 8944-II (generated in the current study, 1 to 10K) and HRP-conjugated anti-rabbit IgG. For binding of S100A proteins to RAGE, calcium-containing TBS buffer (7 mM Tris, 150 mM, 2 mM CaCl₂), 0.1 or 1% BSA) was initially used. For binding of S100A proteins to other receptors, zinc-containing TBS buffer (7 mM Tris, 150 mM, 2 mM CaCl₂, 50 μM ZnSO₄, 1% BSA) was employed to increase the signal. The data are representative of at least two independent experiments.

Assessment of the Effect of S100A8/A9 Antibody (6C8) on Erythrocyte and Myeloid Differentiation of Hematopoietic Progenitor Cells from MDS Patients

Peripheral blood was obtained from patients diagnosed with MDS after signing informed consent in accordance with the Declaration of Helsinki and approved by the Albert Einstein College of Medicine Institutional Review Boards. Mononuclear cells were isolated by Ficoll-Hypaque density gradient separation and cultured in methylcellulose media (Methocult, GF #H4434, Stemcell Technologies) for 14 days in the presence or absence of 2 μg/ml 6C8. Cells were extracted with 2% FBS-containing PBS, washed with ice-cold PBS once, and then labeled with antibody-fluorophore conjugates. CD71-FITC (eBioscience-11-0719-42) and CD235 (Glycophorin)-PerCp-Cy5 (BioLegend-306614) double-positive cells were gated to score erythroid cells. Myelomonocytic cells were quantified by gating double-positive CD14-Pacific Blue (BioLegend-325616) and CD11b-PE-Cy7 (BioLegend-301412) cells.

Induction of T Regulatory Cells (Treg) from PBMC by CD3 and CD28 Antibodies

Cryopreserved human normal PBMC (peripheral blood mononuclear cells, PBMC-C10M, HumanCells Biosciences) were thawed into RPMI 1640 medium containing 20 mM HEPES, 10% fetal bovine serum, 100 U/ml penicillin, and 100 ug/ml streptomycin at a concentration of one million cells/ml. After 4-hour recovery, the cells were activated by 0.1 μg/ml CD3 antibody (#300331, Biolegend) and 0.2 μg/ml CD28 (#302933, Biolegend) in the presence or absence of human S100A8/A9 recombinant protein (2.5 μg/ml) and/or neutralizing antibody 6C8-mIgG2a (5 ug/ml) for 5 days. The presence of regulatory T cells (CD25 Foxp³⁺) was quantitated by routine flow cytometry using common procedures of fixation and permeabilization reagents. CD25 surface marker was labeled with CD25/IL-2R-BV421 (#564033, BD Biosciences), while intracellular marker FOXP3 was stained with FoxP3-Alexa Fluor® 647 (#561184, BD Biosciences). Statistical analysis was performed with one-way matched ANOVA from 5 independent experiments (GraphPad Prism).

### Screening of Humanized 8H6 Antibodies

ELISA plate was coated with CD69 (50 μl, 2.5 ug/ml) overnight. Binding of S100A8/A9-Sino (1 ug/ml) to CD69 was performed in the presence or absence of humanized 8H6 antibodies with 8H6-HC (human chimera) as a positive control and human serum IgG as a negative control in TBS buffer supplemented with 1% Tween 20. Three different antibody concentrations (10, 2.5, and 1.25 μg/ml) were chosen for more accurate comparison between antibodies. The plate-bound S100A8/A9 protein was detected with 6C8-mIgG2a followed by HRP-conjugated anti-Mouse IgG. The data are one representative of three independent experiments.

### Example 2

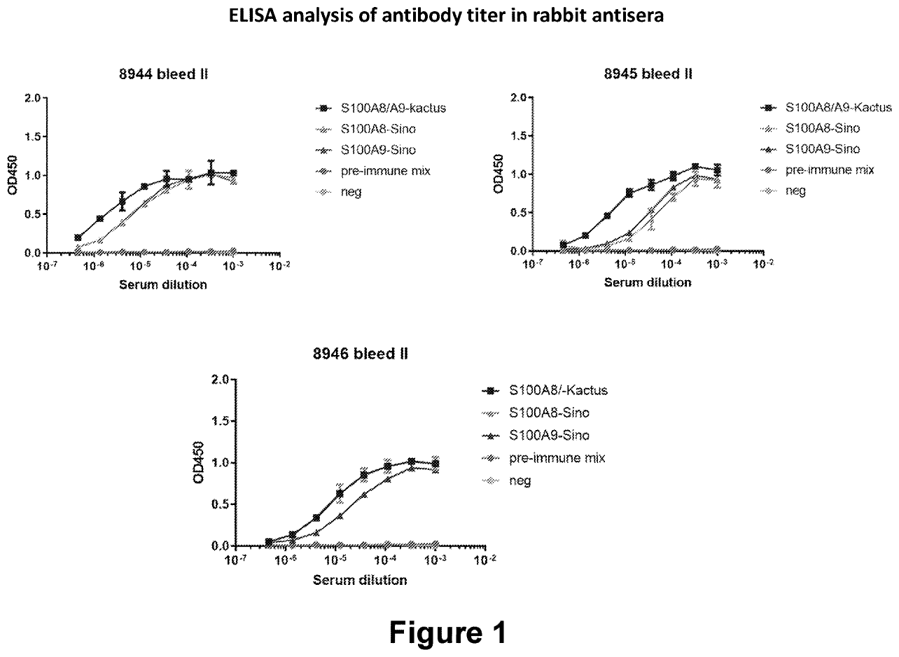

As shown in FIG. 1, to identify the best rabbit for B-cell cloning and generate high titer antiserum for S100A8 protein, S100A9 protein, and S100A8/A9 heterodimer, three rabbits were injected four times with human recombinant S1008/A9-Kactus. The second test bleed was collected 7 weeks after the first immunization. The affinity and specificity of antiserum were assessed by ELISA. As indicated in FIG. 1, rabbit 8944 showed the highest titer of antibody for S100A8/A9-Kactus. The antisera were also highly reactive to S100A8-Sino and S100A9-Sino. The antiserum from rabbit 8894 (namely 8894-II) was used for the ELISA studies for characterization of monoclonal antibodies described below. Rabbit 8944 was injected with S100A8/A9-Kactus again and sacrificed for splenocytes. The results indicated that rabbit 8944 can be used for B-cell cloning and generation of rabbit antiserum 8944-II. Rabbit antiserum 8944-II was extensively used for characterization of monoclonal antibodies.

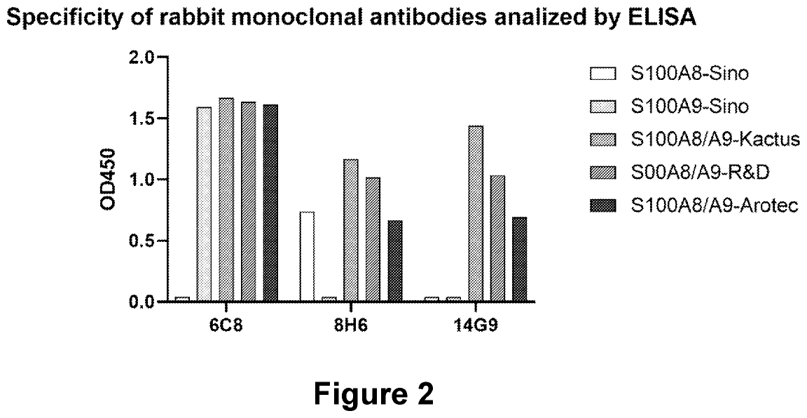

Next, to characterize the specificity of rabbit monoclonal antibodies, splenocytes from rabbit 8944 were sorted, cloned, and screened using the supernatant of B-cells culture. Antibody genes were cloned from the selected B-cells, and recombinant antibodies were further screened for the affinity and specificity for S100A8, S100A9, and S100A8/A9. Three rabbit monoclonal antibodies, 6C8, 8H6, and 14G9, were chosen for further analysis. As shown in FIG. 2, 6C8 recognizes S100A9-Sino, but does not recognize S100A8-Sino. 8H6, on the other hand, recognizes S100A8-Sino but does not recognize S100A9-Sino. 14G9 recognizes neither S100A8-Sino nor S100A9-Sino. All these three antibodies react to S100A8/A9-Kactus. They also recognize S100A8/A9-R&D and S100A8/A9-Arotec. Unlike S100A8/A9-Kactus, S100A8/A9-R&D was not His-tagged, and S100A8/A9-Arotec was purified from human neutrophil. 6C8 has a higher affinity for S100A8/A9 than 8H6 and 14G9. The results indicate that the monoclonal antibodies generated above have unique specificity for S100A8 protein, S100A9 protein, and S100A8/A9 heterodimer.

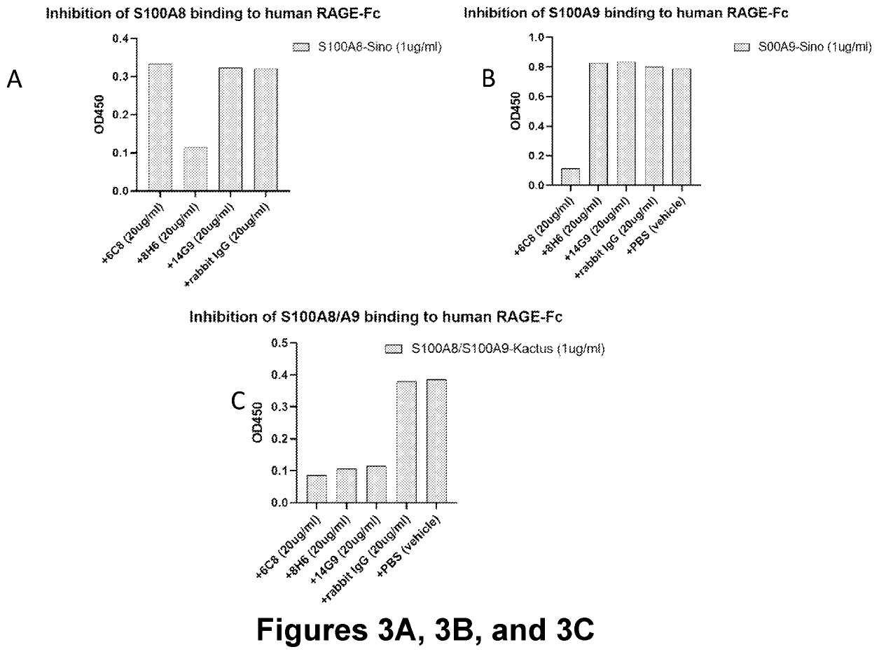

FIG. 3 shows inhibition of binding of S100A8, S100A9, and S100A8/A9 to RAGE-Fc by rabbit monoclonal antibodies. 8H6 suppresses S100A8-Sino binding to RAGE-Fc, 6C8 blocks S100A9-Sino binding to RAGE-Fc, but 14G9 inhibits neither. All three antibodies suppress binding of S100A8/A9-Kactus binding to RAGE-Fc. Rabbit IgG served as a negative control in these experiments. The pattern of inhibition matches exactly the specificity of these antibodies to the individual protein. The results indicate that all three monoclonal antibodies are neutralizing or blocking antibodies of the S100A protein they react with and that all three monoclonal antibodies block S100A8/A9, which is physiologically and pathologically dominant.

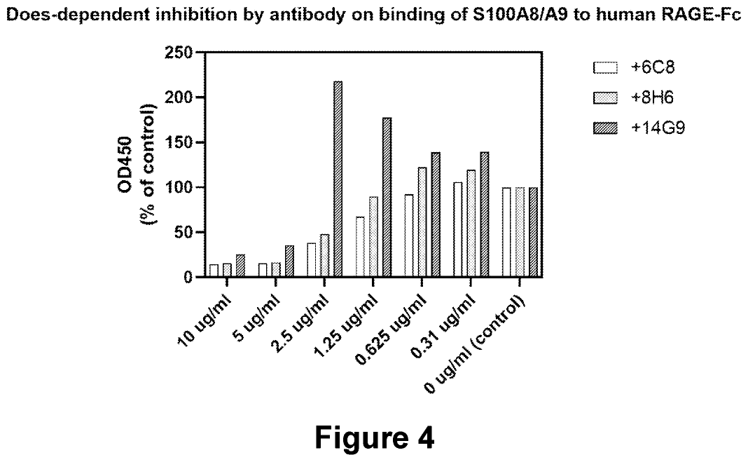

FIG. 4 shows a dose-dependent inhibition of S100A8/A9 binding to RAGE-Fc by the antibodies. Does-dependent inhibition of the binding was observed for all three monoclonal antibodies. While both 6C8 and 8H6 exhibited ~50% inhibition of the binding at 2.5 μg/ml, 14G9 displayed strong inhibition at 5 μg/ml but augmentation of binding at 2.5 μg/ml when the S100A8/A9-Kactus concentration was 1 μg/ml. The underlying mechanism for the augmented binding by the low concentrations of 14G9 was not clear. The results indicate that monoclonal antibodies, 6C8 and 8H6, can efficiently block or neutralize S100A8/A9-Kactus binding to RAGE-Fc.

To generate mouse chimera antibodies, 6C8-mIgG2a and 8H6-mIgG2a from rabbit antibody 6C8 and 8H6, the rabbit constant regions of 6C8 and 8H6 were replaced by the mouse IgG2a constant regions. This allowed more accurate and broad measurement of their inhibition on binding of the S100A proteins to their receptors. For assessment of the binding inhibition by rabbit antibodies, mouse anti-His antibody has been used. However, not all S100A proteins are His-tagged. Also, in the presence of zinc ion, which is required for binding of S100A proteins to some receptors, the mouse-anti-His antibody does not recognize His-Tag anymore.

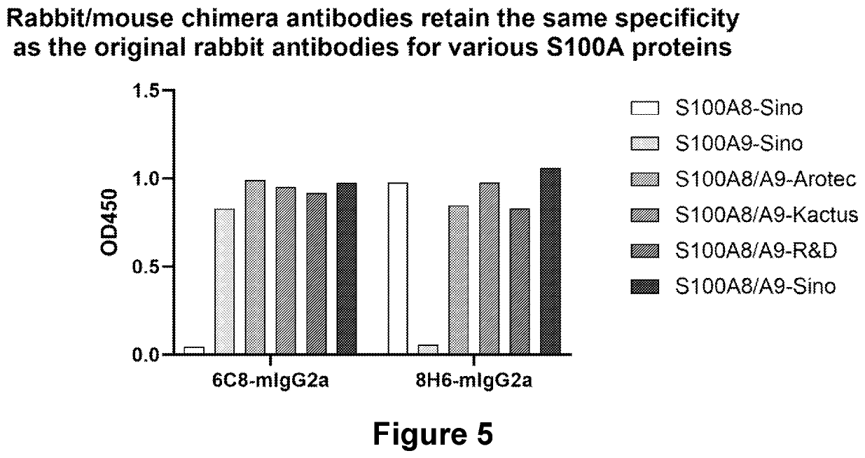

To characterize specificity of mouse chimera antibodies 6C8-mIgG2a and 8H6-mIgG2a, affinity of mouse chimera antibodies, 6C8-mIgG2a and 8H6-mIgG2a for S100A8-Sino, S100A9-Sino, and S100A8/A9 was assessed by ELISA. As shown in FIG. 5, both 6C8-mIgG2a and 8H6-mIgG2a retained their respective specificity and had approximately the same affinity for S100A8/A9 proteins obtained from four different sources. The results indicated that 6C8-mIgG2a and 8H6-mIgG2a retain their high specificity and have high affinity for S100A8/A9.

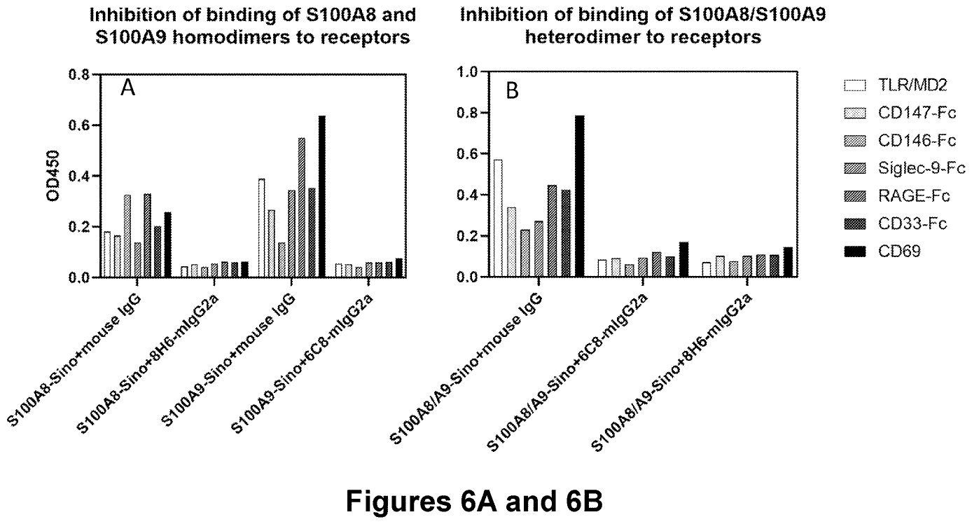

FIG. 6 shows inhibition of binding of human S100A8, S100A9, and S100A8/A9 to their receptors by 6C8-mIgG2a and 8H6-mIgG2a in the presence of zinc ions. The inhibition assay was performed in the presence of zinc ion (50 μM) in addition to calcium ion (2 mM) to increase binding of S100A proteins to some of their receptors. A total of seven receptors that were known to bind S100A proteins (except Siglec-9) were chosen for the assay. Interaction between S100A proteins and Siglec-9 was not established before, but Siglec-9 and CD33 (Siglec-3) belong to the same Siglec family and are highly expressed in myeloid cells. The receptor proteins utilized are soluble extracellular domains of the receptors often fused to a constant fragment of human IgG1. In this experiment, S100A proteins at 1 μg/ml and antibodies at 10 μg/ml were employed, and Mouse IgG served as a negative control. Without exception, marked suppression of S100A8 binding to the receptors was observed for 8H6-mIgG2a, while strong suppression of S100A9 binding to the receptors was observed for 6C8-mIgG2a. Marked inhibition of S100A8/A9 binding to the receptors was found for both antibodies. The results indicate that 6C8-mIgG2a blocks S100A9 binding to all receptors tested, 8H6-mIgG2a blocks S100A8 binding to all receptors tested, and both 6C8-mIgG2a and 8H6-mIgG2a block S100A8/A9 binding to all receptors tested.

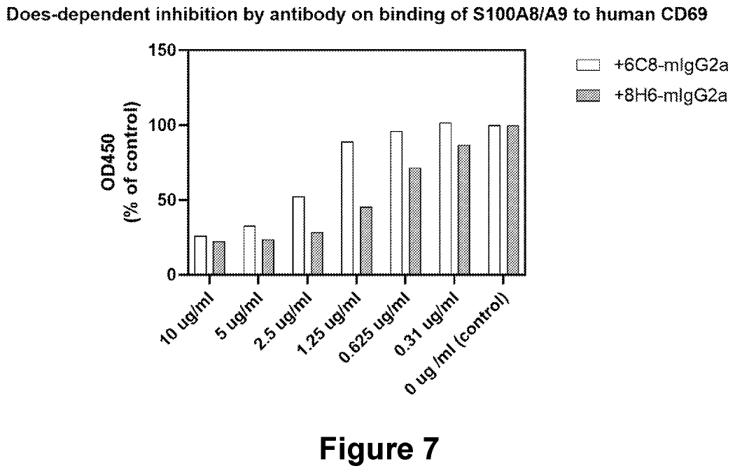

FIG. 7 shows dose-dependent inhibition of S100A8/A9-Sino binding to CD69 by 6C8-mIgG2a and 8H6-mIgG2a. Interaction between CD69 and S1008/A9 dampens monocyte dynamics and induces regulatory T-cell differentiation. Based on the critical role of S100A8/A9 in immunosuppression, dose-dependent inhibition of S100A8/A9 binding to CD69 by both chimera antibodies was examined. Again, a dose-dependent binding inhibition was observed for both 6C8-mIgG2a and 8H6-mIgG2a. To achieve approximate 50% inhibition, 2.5 μg/ml 6C8-mIgG2a or 1.25 μg/ml 8H6-mIgG2a was required in the presence of 1 μg/ml of S100A8/A9-Sino. Therefore, both chimera antibodies efficiently block the binding of S100A8/A9-Sino to CD69, 8H6-mIgG2a appeared to have higher capacity than 6C8-mIgG2a for the inhibition. The results indicate that both 6C8-mIgG2a and 8H6-mIgG2a can efficiently block the binding of S100A8/A9-Sino to CD69, with 8H6-mIgG2a having a higher block capacity than 6C8-mIgG2a.

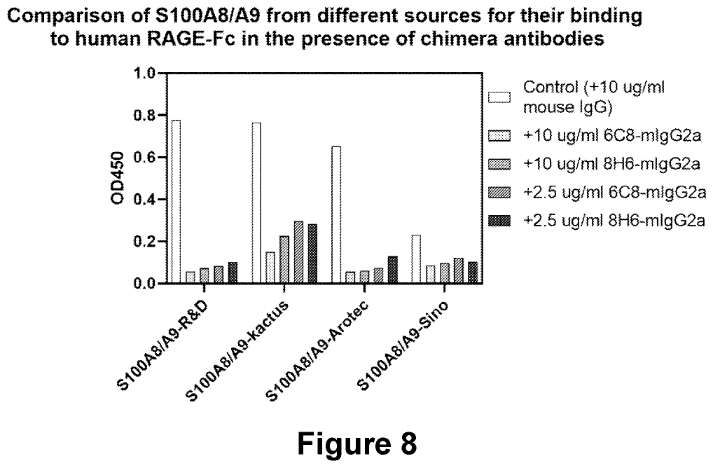

FIG. 8 shows a comparison of S100A8/A9 from different sources for their binding to RAGE-Fc in the presence and absence of 6C8-mIgG2a and 8H6-mIgG2a. S100A8/A9 obtained from four different companies are different from each other either in their tagging or source of expression. S100A8/A9-R&D is expressed in E. coli and not His-tagged. S100A8/A9-Kactus is also produced in E. coli but His-tagged. S100A8/A9-Arotec was purified from human neutrophils and at least partially phosphorylated. S100A8/A9-Sino was expressed in baculovirus-insect cells with both His- and Flag-tags. A side-by-side comparison indicates that S100A8/A9-Sino was less efficient in binding RAGE-Fc under the same condition. Also, there was some non-inhibitable/non-specific binding of S100A8/A9-Kactus to RAGE-Fc. Binding of either S100A8/A9-R&D or “physiological” S100A8/A9-Arotec to RAGE-Fc was markedly suppressed at an antibody concentration of 2.5 μg/ml for 1 μg/ml S100A8/A9. At an antibody concentration of 2.5 μg/ml, at least 50% inhibition was achieved for binding of S100A8/A9-Kactus and S100A8/A9-Sino to RAGE-Fc. The results indicate that 6C8-mIgG2a and 8H6-mIgG2a strongly suppress binding of S100A8/A9 to RAGE-Fc regardless of the sources of the protein.

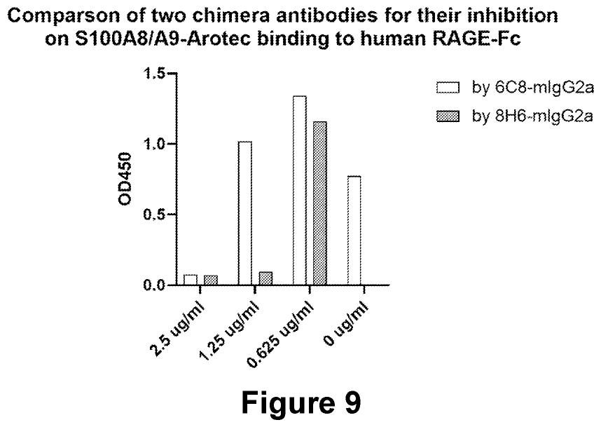

FIG. 9 shows inhibition of S100A8/A9-Arotec binding to RAGE-Fc by lower concentrations of chimera antibodies. A complete inhibition of the physiological S100A8/A9-Arotec binding (1 μg/ml or 45 nM based upon the molecular weight or 22 kDa) to RAGE-Fc was achieved by 6C8-mIgG2a at 2.5 μg/ml (17 nM based upon the molecular weight of 150 kDa) or 8H6-mIgG2a at 1.25 μg/ml (8.3 nM). The neutralizing capacity of 8H6-mIgG2a exceeded the expected molar ratio of one antibody for two antigens. It is unclear if this super neutralization capacity of 8H6-mIgG2a is due to partial degradation and/or unique structure of S100A8/A9-Arotec protein, or unique interaction between antibody and this protein. For both chimera antibodies, there was a steep drop in inhibition when the antibody concentration was reduced in half, indicating an extremely high affinity of antibodies for S100A8/A9-Arotec. The results indicate that 8H6-mIgG2a has a higher than expected capacity to block S100A8/A9-Arotec binding to RAGE-Fc.

To investigate the effects of S100A8/A9 antibody-6C8 on erythrocyte differentiation, mononuclear cells separated from blood from MDS patients were grown on methylcellulose for 14 days in the presence or absence of 6C8 (2 μg/ml). Colonies were subjected to FACS analysis for erythroid differentiation using CD71 and CD235a antibodies. Increased expression of CD71, CD235a or both were noted in the presence of 6C8 in these cases. A block in erythroid differentiation is the hallmark of MDS and AML and leads to anemia in patients. The results indicate that S100A8/A9 antibody-6C8 increased erythrocyte differentiation of MDS hematopoietic progenitor cells.

To investigate the effects of S100A8/A9 antibody-6C8 on myeloid differentiation, mononuclear cells separated from blood from MDS patients were grown on methylcellulose for 14 days in the presence or absence of 6C8 (2 μg/ml). Colonies were subjected to FACS analysis for myeloid differentiation using CD11b and CD14 antibodies. Increased expression of CD11b, CD14 or both were noted in the presence of 6C8 in these cases. A block in myeloid differentiation is the hallmark of MDS and AML and leads to neutropenia in patients. The results indicate S100A8/A9 antibody-6C8 increased myeloid differentiation of MDS hematopoietic progenitor cells.

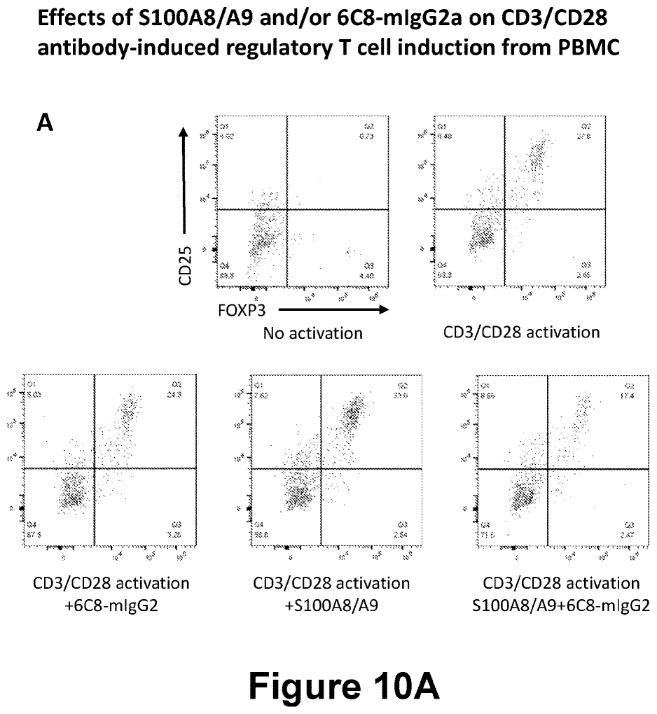

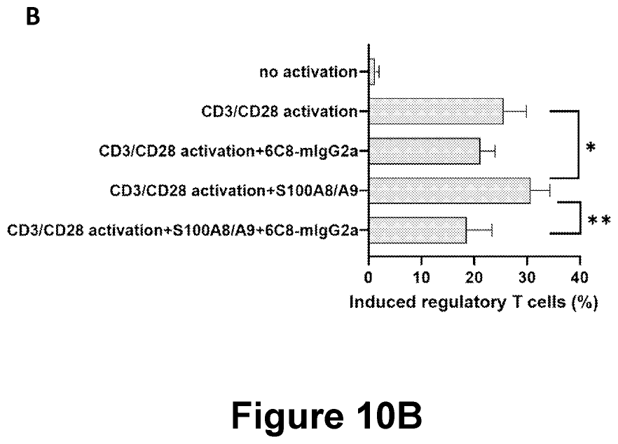

FIGS. 10A and 10B show the impacts of S100A8/A9 protein, neutralizing antibody 6C8-mIgG2a, or in combination on CD3/CD28 antibody-induction of Treg cells (CD25⁺Foxp³⁺) from PBMC. While FIG. 10A shows one representative experiment, FIG. 10B displays the mean±SEM of five independent experiments. There was no Treg cells if T cells were not activated by CD3/CD28 antibodies. There was a significant increase in Treg cell percentages when S100A8/A9 at 2.5 μg/ml (a concentration ~2 to 4-fold higher than the physiological range in plasma) was added to medium, indicating that S100A8/A9 increases Treg cell induction. There was a more significant decrease in Treg cells when 6C8-mIgG2a was added on top of the recombinant protein, indicating that neutralization of S100A8/A9 markedly decreased Treg cell induction. These results indicate that 6C8-mIgG2a can reverse the S100A8/A9-enhanced Treg cell induction when T cells are activated.

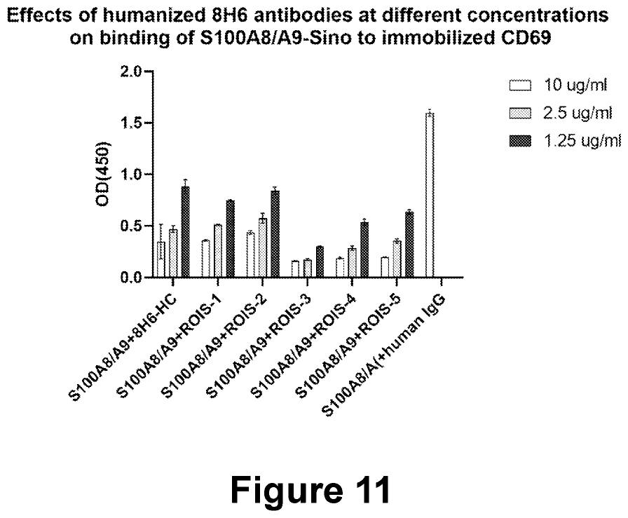

FIG. 11 shows the inhibition of S100A8/A9 binding to CD69 by humanized 8H6 antibodies. The 8H6 humanization was contracted to a commercial company. Human IgG4 Fc was chosen since it binds poorly to the effector cells. Also, a point mutation in the Fc domain, S228P, was introduced to prevent Fab arm exchange of antibodies. A total of 16 clones was initially designed, and the best five antibodies were produced. A human chimera antibody (8H6-HC) was used as a positive control to measure the neutralization capability of five humanized antibodies. We named these five humanized antibodies ROIS-1 to ROIS-5. As indicated in FIG. 11, three of five clones (ROIS-3, ROIS-4, and ROIS-5) had better neutralizing potential than 8H6-HC. The affinity of these humanized antibodies for S100A8/A9-Sino, KD (M), was also determined by Bio-layer interferometry (BLI) and was in order of ROIS-3 (4.77E-11)>ROIS-2 (6,08E-10)>ROIS-5 (6.23E-10)>ROIS-1 (7.89E-10)>8H6-HC (1.37E-9)>ROIS-4 (1.76E-9). These results indicate that 8H6 has been successfully humanized, with ROIS-3 having the highest neutralization potential and the highest affinity for S100A8/A9.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. An isolated antibody or antigen-binding fragment thereof that binds specifically to a S100A8/S100A9 hetero-oligomer, wherein the antibody or antigen-binding fragment thereof comprises:
(a) three heavy chain complementarity determining regions (HCDRs) (HCDR1, HCDR2, and HCDR3) contained within a heavy chain variable region (HCVR) having the amino acid sequence of SEQ ID NO: 1; and three light chain CDRs (LCDR1, LCDR2, and LCDR3) contained within a light chain variable region (LCVR) having the amino acid sequence of SEQ ID NO: 2;
(b) HCDR1, HCDR2, and HCDR3 of a HCVR having the amino acid sequence of SEQ ID NO: 11; and LCDR1, LCDR2, and LCDR3 of a LCVR having the amino acid sequence of SEQ ID NO: 12;
(c) HCDR1, HCDR2, and HCDR3 of a HCVR having the amino acid sequence of SEQ ID NO: 21; and LCDR1, LCDR2, and LCDR3 of a LCVR having the amino acid sequence of SEQ ID NO: 22;
(d) HCDR1, HCDR2, and HCDR3 of a HCVR having the amino acid sequence of SEQ ID NO: 31; and LCDR1, LCDR2, and LCDR3 of a LCVR having the amino acid sequence of SEQ ID NO: 32;
(e) HCDR1, HCDR2, and HCDR3 of a HCVR having the amino acid sequence of SEQ ID NO: 41; and LCDR1, LCDR2, and LCDR3 of a LCVR having the amino acid sequence of SEQ ID NO: 42;
(f) HCDR1, HCDR2, and HCDR3 of a HCVR having the amino acid sequence of SEQ ID NO: 51; and LCDR1, LCDR2, and LCDR3 of a LCVR having the amino acid sequence of SEQ ID NO: 52;
(g) HCDR1, HCDR2, and HCDR3 of a HCVR having the amino acid sequence of SEQ ID NO: 61; and LCDR1, LCDR2, and LCDR3 of a LCVR having the amino acid sequence of SEQ ID NO: 62; or
(h) HCDR1, HCDR2, and HCDR3 of a HCVR having the amino acid sequence of SEQ ID NO: 71; and LCDR1, LCDR2, and LCDR3 of a LCVR having the amino acid sequence of SEQ ID NO: 72.

2. The antibody or antigen-binding fragment thereof of claim 1, comprising HCDR1, HCDR2, HCDR3, LCDR1, LCDR2, and LCDR3 sequences respectively set forth in a CDR sequence set selected from the group consisting of SEQ ID NOs: 3-8, 13-18, 23-28, 33-38, 43-48, 53-58, 63-68, and 73-78.

3. The antibody or antigen-binding fragment thereof of, claim 1 comprising:
(a) a HCVR comprising an amino acid sequence with at least 75% identity to the amino acid sequence of SEQ ID NO: 1 or comprising the amino acid sequence of SEQ ID NO: 1; and a LCVR comprising an amino acid sequence with at least 75% identity to the amino acid sequence of SEQ ID NO: 2 or comprising the amino acid sequence of SEQ ID NO: 2;
(b) a HCVR comprising an amino acid sequence with at least 75% identity to the amino acid sequence of SEQ ID NO: 11 or comprising the amino acid sequence of SEQ ID NO: 11; and a LCVR comprising an amino acid sequence with at least 75% identity to the amino acid sequence of SEQ ID NO: 12 or comprising the amino acid sequence of SEQ ID NO: 12;
(c) a HCVR comprising an amino acid sequence with at least 75% identity to the amino acid sequence of SEQ ID NO: 21 or comprising the amino acid sequence of SEQ ID NO: 21; and a LCVR comprising an amino acid sequence with at least 75% identity to the amino acid sequence of SEQ ID NO: 22 or comprising the amino acid sequence of SEQ ID NO: 22′
(d) a HCVR comprising an amino acid sequence with at least 75% identity to the amino acid sequence of SEQ ID NO: 31 or comprising the amino acid sequence of SEQ ID NO: 31; and a LCVR comprising an amino acid sequence with at least 75% identity to the amino acid sequence of SEQ ID NO: 32 or comprising the amino acid sequence of SEQ ID NO: 32;
(e) a HCVR comprising an amino acid sequence with at least 75% identity to the amino acid sequence of SEQ ID NO: 41 or comprising the amino acid sequence of SEQ ID NO: 41; and a LCVR comprising an amino acid sequence with at least 75% identity to the amino acid sequence of SEQ ID NO: 42 or comprising the amino acid sequence of SEQ ID NO: 42;
(f) a HCVR comprising an amino acid sequence with at least 75% identity to the amino acid sequence of SEQ ID NO: 51 or comprising the amino acid sequence of SEQ ID NO: 51; and a LCVR comprising an amino acid sequence with at least 75% identity to the amino acid sequence of SEQ ID NO: 52 or comprising the amino acid sequence of SEQ ID NO: 52;
(g) a HCVR comprising an amino acid sequence with at least 75% identity to the amino acid sequence of SEQ ID NO: 61 or comprising the amino acid sequence of SEQ ID NO: 61; and a LCVR comprising an amino acid sequence with at least 75% identity to the amino acid sequence of SEQ ID NO: 62 or comprising the amino acid sequence of SEQ ID NO: 62; or
(h) a HCVR comprising an amino acid sequence with at least 75% identity to the amino acid sequence of SEQ ID NO: 71 or comprising the amino acid sequence of SEQ ID NO: 71; and a LCVR comprising an amino acid sequence with at least 75% identity to the amino acid sequence of SEQ ID NO: 72 or comprising the amino acid sequence of SEQ ID NO: 72.

4. The antibody or antigen-binding fragment thereof of, claim 1, comprising a HCVR and a LCVR that comprise a HCVR and LCVR amino acid sequence pair of SEQ ID NOs: 1-2, 11-12, 21-22, 31-32, 41-42, 51-52, 61-62, or 71-72.

5. The antibody or antigen-binding fragment thereof of, claim 1, comprising a heavy chain (HC) and a light chain (LC) that comprise a HC and LC amino acid sequence pair of SEQ ID NOs: 9-10, 19-20, 29-30, 39-40, 49-50, 59-60, 69-70, or 79-80.

6. The antibody or antigen-binding fragment thereof of, claim 1, wherein the antibody or antigen-binding fragment thereof is a 6C8 antibody, a 8H6 antibody, or a 14G9 antibody.

7. The antibody or antigen-binding fragment thereof of, claim 1, wherein the S100A8/S100A9 hetero-oligomer comprises a S100A8/S100A9 heterodimer.

8. The antibody or antigen-binding fragment thereof of, claim 1, wherein the S100A8 protein is a human S100A8 protein and the S100A9 protein is a human S100A9 protein.

9. (canceled)

10. The antibody or antigen-binding fragment thereof of, claim 1, wherein the antibody is a monoclonal or polyclonal antibody.

11. The antibody or antigen-binding fragment thereof of, claim 1, wherein the antibody is a bivalent or bispecific antibody.

12. (canceled)

13. The antibody or antigen-binding fragment thereof of, claim 1, wherein the antibody is a chimeric antibody, a humanized antibody, or a humanized monoclonal antibody.

14. The antibody or antigen-binding fragment thereof of, claim 1, wherein the antibody or antigen-binding fragment thereof comprises a single-chain antibody, Fab or Fab2 fragment.

15. The antibody or the antigen-binding fragment thereof of, claim 1, wherein the antibody or antigen-binding fragment thereof comprises a variant Fc constant region.

16. The antibody or antigen-binding fragment thereof of, claim 1, wherein the antibody or antigen-binding fragment thereof is detectably labeled or conjugated to a toxin, a therapeutic agent, a polymer, a receptor, an enzyme, or a receptor ligand, preferably wherein the polymer is polyethylene glycol (PEG).

17. A nucleic acid molecule encoding a polypeptide chain of the antibody or antigen-binding fragment thereof of claim 1.

18. (canceled)

19. (canceled)

20. (canceled)

21. A pharmaceutical composition comprising the antibody or antigen-binding fragment thereof of claim 1; and optionally a pharmaceutically acceptable carrier or excipient.

22. (canceled)

23. (canceled)

24. A method of treating a disease or disorder in a subject in need thereof, comprising administering to the subject the antibody or antigen-binding fragment thereof of, claim 1.

25. The method of claim 24, wherein the disease or disorder comprises leukemia, myelodysplastic syndromes (MDS), or an inflammatory and autoimmune disease.

26. The method of claim 25, wherein the leukemia is acute lymphoblastic leukemia (ALL), acute myeloid leukemia (AML), chronic lymphocytic leukemia (CLL), chronic myeloid leukemia (CML), or chronic myelomonocytic leukemia (CMML).

27. The method of claim 24, wherein the inflammatory and autoimmune disease is non-neoplastic inflammation

28. The method of claim 24, wherein further comprising administering to the subject an additional therapeutic agent or therapy.

29. The method of claim 28, wherein the additional therapeutic agent or therapy comprises at least one of daunorubicin, doxorubicin, and cytarabine.

30. The method of claim 28, wherein the antibody or antigen-binding fragment thereof is administered before, after, or concurrently with the additional therapeutic agent or therapy.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)


SEQUENCE LISTING (160 sequences)

SEQ ID NO: 1 - protein, 117 aa, synthetic construct
QEQLVESGGGLVTLGGSLKLSCKASGIDFSIYGISWVRQAPGKGLEWIAYIYPDYSTTDYASWVNGRFTISFDNAQNTVFLEMTSLTAADTATYFCARRYSGYYGLWGPGTLVTVSS

SEQ ID NO: 2 - protein, 112 aa, synthetic construct
ALVMTQTPSSVSAPVGGTVTIKCQASQSISSYLAWYQQKPGQRPKLLIYGASSLESGVPSRFKGSGSGTEYTLTISDLECDDAATYYCQGAHAGSSADAFGGGTEVVVKGDP

SEQ ID NO: 3 - protein, 5 aa, synthetic construct
IYGIS

SEQ ID NO: 4 - protein, 17 aa, synthetic construct
YIYPDYSTTDYASWVNG

SEQ ID NO: 5 - protein, 8 aa, synthetic construct
RYSGYYGL

SEQ ID NO: 6 - protein, 11 aa, synthetic construct
QASQSISSYLA

SEQ ID NO: 7 - protein, 7 aa, synthetic construct
GASSLES

SEQ ID NO: 8 - protein, 11 aa, synthetic construct
QGAHAGSSADA

SEQ ID NO: 9 - protein, 459 aa, synthetic construct
METGLRWLLLVAVLKGVQCQEQLVESGGGLVTLGGSLKLSCKASGIDFSIYGISWVRQAPGKGLEWIAYIYPDYSTTDYASWVNGRFTISFDNAQNTVFLEMTSLTAADTATYFCARRYSGYYGLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 10 - protein, 235 aa, synthetic construct
MDTRAPTQLLGLLLLWLPGARCALVMTQTPSSVSAPVGGTVTIKCQASQSISSYLAWYQQKPGQRPKLLIYGASSLESGVPSRFKGSGSGTEYTLTISDLECDDAATYYCQGAHAGSSADAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 11 - protein, 120 aa, synthetic construct
CQSLEESGGDLVKPEGSLTLTCKASGFSFSSGCWVCWVRQAPGKGLEWIACIYTGDGDTYYANWARGRFTVSRTSSTTVTLQLTSLTAADTATYFCASPQPGDITYLGFWGPGTLVTVSS

SEQ ID NO: 12 - protein, 116 aa, synthetic construct
DIVMTQTPASVSEPVGGTVTIKCQASQNINSYLSWYQQRPGQPPKLLISLASDLASGVPSRFKGSESGTDYTLTISDLECADAATYYCQGYFADSGSSGRSWAFGGGTEVVVQGDP

SEQ ID NO: 13 - protein, 6 aa, synthetic construct
SGCWVC

SEQ ID NO: 14 - protein, 17 aa, synthetic construct
CIYTGDGDTYYANWARG

SEQ ID NO: 15 - protein, 11 aa, synthetic construct
PQPGDITYLGF

SEQ ID NO: 16 - protein, 11 aa, synthetic construct
QASQNINSYLS

SEQ ID NO: 17 - protein, 7 aa, synthetic construct
LASDLAS

SEQ ID NO: 18 - protein, 15 aa, synthetic construct
QGYFADSGSSGRSWA

SEQ ID NO: 19 - protein, 461 aa, synthetic construct
METGLRWLLLVAVLKGVQCQSLEESGGDLVKPEGSLTLTCKASGFSFSSGCWVCWVRQAPGKGLEWIACIYTGDGDTYYANWARGRFTVSRTSSTTVTLQLTSLTAADTATYFCASPQPGDITYLGFWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 20 - protein, 240 aa, synthetic construct
MDTRAPTQLLGLLLLWLPGARCADIVMTQTPASVSEPVGGTVTIKCQASQNINSYLSWYQQRPGQPPKLLISLASDLASGVPSRFKGSESGTDYTLTISDLECADAATYYCQGYFADSGSSGRSWAFGGGTEVVVQGDPVAPTVLIFPPATDQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 21 - protein, 121 aa, synthetic construct
QEQLVESGGGLVQPEGSLTLTCTASGFSFSSDYYMCWVRQAPGKGLEWIACIGGGYGGSPYYATWAKGRFTISKTSSTAVTLQMTSLTAADTATHFCARGVPGSSCFFDLWGPGTLVTVSS

SEQ ID NO: 22 - protein, 110 aa, synthetic construct
DVAMTQTPASVSEPVGGTVTIKCQASQSIGSSLAWYQQKSGQPPKLLIYDAFDLASGVSSRFKGSGSGTEYTLTISDLECADAATYFCLGGYYDANTFGGGTEVVVKGDP

SEQ ID NO: 23 - protein, 6 aa, synthetic construct
SDYYMC

SEQ ID NO: 24 - protein, 18 aa, synthetic construct
CIGGGYGGSPYYATWAKG

SEQ ID NO: 25 - protein, 11 aa, synthetic construct
GVPGSSCFFDL

SEQ ID NO: 26 - protein, 11 aa, synthetic construct
QASQSIGSSLA

SEQ ID NO: 27 - protein, 7 aa, synthetic construct
DAFDLAS

SEQ ID NO: 28 - protein, 9 aa, synthetic construct
LGGYYDANT

SEQ ID NO: 29 - protein, 463 aa, synthetic construct
METGLRWLLLVAVLKGVQCQEQLVESGGGLVQPEGSLTLTCTASGFSFSSDYYMCWVRQAPGKGLEWIACIGGGYGGSPYYATWAKGRFTISKTSSTAVTLQMTSLTAADTATHFCARGVPGSSCFFDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 30 - protein, 233 aa, synthetic construct
MDTRAPTQLLGLLLLWLPGASCDVAMTQTPASVSEPVGGTVTIKCQASQSIGSSLAWYQQKSGQPPKLLIYDAFDLASGVSSRFKGSGSGTEYTLTISDLECADAATYFCLGGYYDANTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 31 - protein, 121 aa, synthetic construct
NVQLVESGGGLIHPGGSLSLSCKASGFSFSSGCWVCWVRQAPGKGLEWVACIYTGDGDTYYADSVRGRFTISRTSSKTTVYLQMNSLRAEDTAVYYCASPQPGDITYLGFWGQGTLVTVSS

SEQ ID NO: 32 - protein, 116 aa, synthetic construct
DIHMTQSPSSLSASVGDTVTITCQASQNINSYLSWYQQEPGKAPKLLISLASDLASGVPSRFSGSESGTDYTLTISSLQPEDFATYYCQGYFADSGSSGRSWAFGQGTKVEIKRTV

SEQ ID NO: 33 - protein, 6 aa, synthetic construct
SGCWVC

SEQ ID NO: 34 - protein, 17 aa, synthetic construct
CIYTGDGDTYYADSVRG

SEQ ID NO: 35 - protein, 11 aa, synthetic construct
PQPGDITYLGF

SEQ ID NO: 36 - protein, 11 aa, synthetic construct
QASQNINSYLS

SEQ ID NO: 37 - protein, 7 aa, synthetic construct
LASDLAS

SEQ ID NO: 38 - protein, 15 aa, synthetic construct
QGYFADSGSSGRSWA

SEQ ID NO: 39 - protein, 466 aa, synthetic construct
METGLRWLLLVAVLKGVQNVQLVESGGGLIHPGGSLSLSCKASGFSFSSGCWVCWVRQAPGKGLEWVACIYTGDGDTYYADSVRGRFTISRTSSKTTVYLQMNSLRAEDTAVYYCASPQPGDITYLGFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 243 aa, synthetic construct
MDTRAPTQLLGLLLLWLPGARCADIHMTQSPSSLSASVGDTVTITCQASQNINSYLSWYQQEPGKAPKLLISLASDLASGVPSRFSGSESGTDYTLTISSLQPEDFATYYCQGYFADSGSSGRSWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 121 aa, synthetic construct
NVQLVESGGGLIHPGGSLSLSCKASGFSFSSGCWVCWVRQAPGKGLEWVACIYTGDGDTYYADSVKGRFTISRTSSKTTVYLQMNSLRAEDTAVYYCASPQPGDITYLGFWGQGTLVTVSS

SEQ ID NO: 42 - protein, 116 aa, synthetic construct
DIHMTQSPSSLSASVGDTVTITCQASQNINSYLSWYQQKPGKAPKLLISLASDLASGVPSRFSGSESGTDYTLTISSLQPEDFATYYCQGYFADSGSSGRSWAFGQGTKVEIKRTV

SEQ ID NO: 43 - protein, 6 aa, synthetic construct
SGCWVC

SEQ ID NO: 44 - protein, 17 aa, synthetic construct
CIYTGDGDTYYADSVKG

SEQ ID NO: 45 - protein, 11 aa, synthetic construct
PQPGDITYLGF

SEQ ID NO: 46 - protein, 11 aa, synthetic construct
QASQNINSYLS

SEQ ID NO: 47 - protein, 7 aa, synthetic construct
LASDLAS

SEQ ID NO: 48 - protein, 15 aa, synthetic construct
QGYFADSGSSGRSWA

SEQ ID NO: 49 - protein, 466 aa, synthetic construct
METGLRWLLLVAVLKGVQNVQLVESGGGLIHPGGSLSLSCKASGFSFSSGCWVCWVRQAPGKGLEWVACIYTGDGDTYYADSVKGRFTISRTSSKTTVYLQMNSLRAEDTAVYYCASPQPGDITYLGFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 243 aa, synthetic construct
MDTRAPTQLLGLLLLWLPGARCADIHMTQSPSSLSASVGDTVTITCQASQNINSYLSWYQQKPGKAPKLLISLASDLASGVPSRFSGSESGTDYTLTISSLQPEDFATYYCQGYFADSGSSGRSWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 121 aa, synthetic construct
QVSLEESGGDLVQPGGSLRLACVASGFSFSSGCWVCWVRQAPGKGLEWVSCIYTGDGDTYYANSVRGRFTVSRQNSTTSVYLQMNSLRADDTALYFCASPQPGDITYLGFWGPGTLVTVSS

SEQ ID NO: 52 - protein, 116 aa, synthetic construct; identical to SEQ ID NO: 42

SEQ ID NO: 53 - protein, 6 aa, synthetic construct
SGCWVC

SEQ ID NO: 54 - protein, 17 aa, synthetic construct
CIYTGDGDTYYANSVRG

SEQ ID NO: 55 - protein, 11 aa, synthetic construct
PQPGDITYLGF

SEQ ID NO: 56 - protein, 11 aa, synthetic construct
QASQNINSYLS

SEQ ID NO: 57 - protein, 7 aa, synthetic construct
LASDLAS

SEQ ID NO: 58 - protein, 15 aa, synthetic construct
QGYFADSGSSGRSWA

SEQ ID NO: 59 - protein, 466 aa, synthetic construct
METGLRWLLLVAVLKGVQQVSLEESGGDLVQPGGSLRLACVASGFSFSSGCWVCWVRQAPGKGLEWVSCIYTGDGDTYYANSVRGRFTVSRQNSTTSVYLQMNSLRADDTALYFCASPQPGDITYLGFWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 243 aa, synthetic construct; identical to SEQ ID NO: 50

SEQ ID NO: 61 - protein, 121 aa, synthetic construct
QVSLEESGGDLVQPGGSLRLACVASGFSFSSGCWVCWVRQAPGKGLEWVSCIYTGDGDTYYANSVRGRFTVSRKNSTTSVYLQMNSLRADDTALYFCASPQPGDITYLGFWGPGTLVTVSS

SEQ ID NO: 62 - protein, 116 aa, synthetic construct; identical to SEQ ID NO: 42

SEQ ID NO: 63 - protein, 6 aa, synthetic construct
SGCWVC

SEQ ID NO: 64 - protein, 17 aa, synthetic construct
CIYTGDGDTYYANSVRG

SEQ ID NO: 65 - protein, 11 aa, synthetic construct
PQPGDITYLGF

SEQ ID NO: 66 - protein, 11 aa, synthetic construct
QASQNINSYLS

SEQ ID NO: 67 - protein, 7 aa, synthetic construct
LASDLAS

SEQ ID NO: 68 - protein, 15 aa, synthetic construct
QGYFADSGSSGRSWA

SEQ ID NO: 69 - protein, 466 aa, synthetic construct
METGLRWLLLVAVLKGVQQVSLEESGGDLVQPGGSLRLACVASGFSFSSGCWVCWVRQAPGKGLEWVSCIYTGDGDTYYANSVRGRFTVSRKNSTTSVYLQMNSLRADDTALYFCASPQPGDITYLGFWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 243 aa, synthetic construct; identical to SEQ ID NO: 50

SEQ ID NO: 71 - protein, 121 aa, synthetic construct
QVSLEESGGDLVQPGGSLRLACVASGFSFSSGCWVCWVRQAPGKGLEWVSCIYTGDGDTYYANSVRGRFTVSRRNSTTSVYLQMNSLRADDTALYFCASPQPGDITYLGFWGPGTLVTVSS

SEQ ID NO: 72 - protein, 116 aa, synthetic construct; identical to SEQ ID NO: 42

SEQ ID NO: 73 - protein, 6 aa, synthetic construct
SGCWVC

SEQ ID NO: 74 - protein, 17 aa, synthetic construct
CIYTGDGDTYYANSVRG

SEQ ID NO: 75 - protein, 11 aa, synthetic construct
PQPGDITYLGF

SEQ ID NO: 76 - protein, 11 aa, synthetic construct
QASQNINSYLS

SEQ ID NO: 77 - protein, 7 aa, synthetic construct
LASDLAS

SEQ ID NO: 78 - protein, 15 aa, synthetic construct
QGYFADSGSSGRSWA

SEQ ID NO: 79 - protein, 466 aa, synthetic construct
METGLRWLLLVAVLKGVQQVSLEESGGDLVQPGGSLRLACVASGFSFSSGCWVCWVRQAPGKGLEWVSCIYTGDGDTYYANSVRGRFTVSRRNSTTSVYLQMNSLRADDTALYFCASPQPGDITYLGFWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 243 aa, synthetic construct; identical to SEQ ID NO: 50

SEQ ID NO: 81 - DNA, 351 bp, synthetic construct
  source 1..351: mol_type = other DNA
caggagcaactggtggagtctggagggggtctggtcacgcttgggggatccctcaaactctcctgcaaagcctctggaatcgacttcagtatctatggcattagctgggtccgccaggctccagggaaggggctggagtggatcgcatatatttatcctgattatagtaccacagactacgcgagttgggtgaatggccgattcaccatctccttcgacaacgcccagaatacggtgtttctggaaatgaccagtctgacagccgcggacacggccacctatttctgtgcaaggcgttatagtggttattatggcttgtggggcccaggcaccctggtcaccgtctcctca

SEQ ID NO: 82 - DNA, 336 bp, synthetic construct
  source 1..336: mol_type = other DNA
gcccttgtgatgacccagactccatcctccgtgtctgcacctgtgggaggcacagtcaccatcaagtgccaggccagtcagagcattagtagctacttagcctggtatcaacagaaaccagggcagcgtcccaagctcctgatctatggtgcatccagtctggagtctggggtcccatcgcggttcaaaggcagtggatctgggacagagtacactctcaccatcagcgacctggagtgtgacgatgctgccacttactactgtcaaggtgctcatgcaggtagtagtgctgatgctttcggcggagggaccgaggtggtggtcaaaggtgatcca

SEQ ID NO: 83 - DNA, 15 bp, synthetic construct
  source 1..15: mol_type = other DNA
atctatggcattagc

SEQ ID NO: 84 - DNA, 51 bp, synthetic construct
  source 1..51: mol_type = other DNA
tatatttatcctgattatagtaccacagactacgcgagttgggtgaatggc

SEQ ID NO: 85 - DNA, 24 bp, synthetic construct
  source 1..24: mol_type = other DNA
cgttatagtggttattatggcttg

SEQ ID NO: 86 - DNA, 30 bp, synthetic construct
  source 1..30: mol_type = other DNA
caggccagtcagagcattagtagctactta

SEQ ID NO: 87 - DNA, 18 bp, synthetic construct
  source 1..18: mol_type = other DNA
ggtgcatccagtctggag

SEQ ID NO: 88 - DNA, 33 bp, synthetic construct
  source 1..33: mol_type = other DNA
caaggtgctcatgcaggtagtagtgctgatgct

SEQ ID NO: 89 - DNA, 1380 bp, synthetic construct
  source 1..1380: mol_type = other DNA
atggagactgggctgcgctggcttctcctggtcgctgtgctcaaaggtgtccagtgtcaggagcaactggtggagtctggagggggtctggtcacgcttgggggatccctcaaactctcctgcaaagcctctggaatcgacttcagtatctatggcattagctgggtccgccaggctccagggaaggggctggagtggatcgcatatatttatcctgattatagtaccacagactacgcgagttgggtgaatggccgattcaccatctccttcgacaacgcccagaatacggtgtttctggaaatgaccagtctgacagccgcggacacggccacctatttctgtgcaaggcgttatagtggttattatggcttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 90 - DNA, 708 bp, synthetic construct
  source 1..708: mol_type = other DNA
atggacacgagggcccccactcagctgctggggctcctgctgctctggctcccaggtgcccgatgtgcccttgtgatgacccagactccatcctccgtgtctgcacctgtgggaggcacagtcaccatcaagtgccaggccagtcagagcattagtagctacttagcctggtatcaacagaaaccagggcagcgtcccaagctcctgatctatggtgcatccagtctggagtctggggtcccatcgcggttcaaaggcagtggatctgggacagagtacactctcaccatcagcgacctggagtgtgacgatgctgccacttactactgtcaaggtgctcatgcaggtagtagtgctgatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 91 - DNA, 360 bp, synthetic construct
  source 1..360: mol_type = other DNA
tgtcagtcgttggaggagtccgggggagacctggtcaagcctgagggatccctgacactcacctgcaaagcctctggattctccttcagtagcggttgctgggtatgctgggtccgccaggctccagggaaggggttggagtggatcgcatgtatttatactggtgatggtgacacttactacgcgaactgggcgagaggccgattcaccgtctcccgtacctcgtcgaccacggtgactctgcaattgaccagtctgacagccgcggacacggccacctatttctgtgcgagtccacagcctggtgacattacttatttggggttctggggcccgggcaccctggtcaccgtctcctca

SEQ ID NO: 92 - DNA, 348 bp, synthetic construct
  source 1..348: mol_type = other DNA
gacattgtgatgacccagactccagcctccgtgtctgaacctgtgggaggcacagtcaccatcaagtgccaggccagtcagaacattaatagctacttatcctggtatcagcagagaccagggcagcctcccaagctcctgatctcactggcatccgatctggcatctggggtcccatcgcggtttaaaggcagtgaatctgggacagactatactctcaccatcagcgacctggagtgtgccgatgctgccacttactactgtcaaggctattttgctgatagtggtagtagtggtcgttcgtgggctttcggcggcgggaccgaggtggtggtccaaggtgatcca

SEQ ID NO: 93 - DNA, 18 bp, synthetic construct
  source 1..18: mol_type = other DNA
agcggttgctgggtatgc

SEQ ID NO: 94 - DNA, 51 bp, synthetic construct
  source 1..51: mol_type = other DNA
tgtatttatactggtgatggtgacacttactacgcgaactgggcgagaggc

SEQ ID NO: 95 - DNA, 33 bp, synthetic construct
  source 1..33: mol_type = other DNA
ccacagcctggtgacattacttatttggggttc

SEQ ID NO: 96 - DNA, 33 bp, synthetic construct
  source 1..33: mol_type = other DNA
caggccagtcagaacattaatagctacttatcc

SEQ ID NO: 97 - DNA, 21 bp, synthetic construct
  source 1..21: mol_type = other DNA
ctggcatccgatctggcatct

SEQ ID NO: 98 - DNA, 45 bp, synthetic construct
  source 1..45: mol_type = other DNA
caaggctattttgctgatagtggtagtagtggtcgttcgtgggct

SEQ ID NO: 99 - DNA, 1386 bp, synthetic construct
  source 1..1386: mol_type = other DNA
atggagactgggctgcgctggcttctcctggtcgctgtgctcaaaggtgtccagtgtcagtcgttggaggagtccgggggagacctggtcaagcctgagggatccctgacactcacctgcaaagcctctggattctccttcagtagcggttgctgggtatgctgggtccgccaggctccagggaaggggttggagtggatcgcatgtatttatactggtgatggtgacacttactacgcgaactgggcgagaggccgattcaccgtctcccgtacctcgtcgaccacggtgactctgcaattgaccagtctgacagccgcggacacggccacctatttctgtgcgagtccacagcctggtgacattacttatttggggttctggggcccgggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 100 - DNA, 723 bp, synthetic construct
  source 1..723: mol_type = other DNA
atggacacgagggcccccactcagctgctggggctcctgctgctctggctcccaggtgccagatgtgctgacattgtgatgacccagactccagcctccgtgtctgaacctgtgggaggcacagtcaccatcaagtgccaggccagtcagaacattaatagctacttatcctggtatcagcagagaccagggcagcctcccaagctcctgatctcactggcatccgatctggcatctggggtcccatcgcggtttaaaggcagtgaatctgggacagactatactctcaccatcagcgacctggagtgtgccgatgctgccacttactactgtcaaggctattttgctgatagtggtagtagtggtcgttcgtgggctttcggcggcgggaccgaggtggtggtccaaggtgatccagttgcacctactgtcctcatcttcccaccagctactgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 101 - DNA, 363 bp, synthetic construct
  source 1..363: mol_type = other DNA
caggagcagctggtggagtccgggggaggcctggtccagcctgagggatccctgacactcacctgcacagcttctggattctccttcagtagcgactactacatgtgctgggtccgccaggctccagggaaggggctggagtggatcgcttgcattggtggtggttatggtggtagcccttactacgcgacctgggcgaaaggccgcttcaccatctccaaaacctcgtcgaccgcggtgactctacaaatgaccagtctgacagccgcggacacggccacccatttctgtgcgcgcggtgtgcctggtagtagttgtttctttgacttgtggggcccaggcaccctggtcaccgtctcctca

SEQ ID NO: 102 - DNA, 330 bp, synthetic construct
  source 1..330: mol_type = other DNA
gatgttgcgatgacccagactccagcctccgtgtctgaacctgtgggaggcacagtcaccatcaagtgccaggccagtcagagcattggtagtagcttagcctggtatcaacaaaaatcaggacagcctcccaagctcctgatctatgatgcatttgatctggcatctggagtctcatcgcggttcaaaggcagtggatctgggacagagtacactctcaccatcagcgacctggagtgtgccgatgctgccacttatttctgtctaggcggctattatgatgctaatacttttggcggagggaccgaggtggtggtcaaaggtgatcca

SEQ ID NO: 103 - DNA, 18 bp, synthetic construct
  source 1..18: mol_type = other DNA
agcgactactacatgtgc

SEQ ID NO: 104 - DNA, 54 bp, synthetic construct
  source 1..54: mol_type = other DNA
tgcattggtggtggttatggtggtagcccttactacgcgacctgggcgaaaggc

SEQ ID NO: 105 - DNA, 33 bp, synthetic construct
  source 1..33: mol_type = other DNA
ggtgtgcctggtagtagttgtttctttgacttg

SEQ ID NO: 106 - DNA, 33 bp, synthetic construct
  source 1..33: mol_type = other DNA
caggccagtcagagcattggtagtagcttagcc

SEQ ID NO: 107 - DNA, 21 bp, synthetic construct
  source 1..21: mol_type = other DNA
gatgcatttgatctggcatct

SEQ ID NO: 108 - DNA, 27 bp, synthetic construct
  source 1..27: mol_type = other DNA
ctaggcggctattatgatgctaatact

SEQ ID NO: 109 - DNA, 1392 bp, synthetic construct
  source 1..1392: mol_type = other DNA
atggagactgggctgcgctggcttctcctggtcgctgtgctcaaaggtgtccagtgtcaggagcagctggtggagtccgggggaggcctggtccagcctgagggatccctgacactcacctgcacagcttctggattctccttcagtagcgactactacatgtgctgggtccgccaggctccagggaaggggctggagtggatcgcttgcattggtggtggttatggtggtagcccttactacgcgacctgggcgaaaggccgcttcaccatctccaaaacctcgtcgaccgcggtgactctacaaatgaccagtctgacagccgcggacacggccacccatttctgtgcgcgcggtgtgcctggtagtagttgtttctttgacttgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 110 - DNA, 702 bp, synthetic construct
  source 1..702: mol_type = other DNA
atggacacgagggcccccactcagctgctggggctcctgctgctctggctcccaggtgccagctgtgatgttgcgatgacccagactccagcctccgtgtctgaacctgtgggaggcacagtcaccatcaagtgccaggccagtcagagcattggtagtagcttagcctggtatcaacaaaaatcaggacagcctcccaagctcctgatctatgatgcatttgatctggcatctggagtctcatcgcggttcaaaggcagtggatctgggacagagtacactctcaccatcagcgacctggagtgtgccgatgctgccacttatttctgtctaggcggctattatgatgctaatacttttggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 111 - DNA, 363 bp, synthetic construct
  source 1..363: mol_type = other DNA
aacgtgcagctggtcgagtctggaggcggcctgatccaccccggcggaagcctctctctgtcctgcaaggcctctggcttttctttctcctccggctgttgggtgtgctgggtgcggcaagctcctggaaaaggcctggaatgggtcgcctgcatctacaccggcgacggcgatacatattacgccgactccgtgagaggcagattcaccatctcccggacctcttccaagaccaccgtgtacctgcagatgaactccctgagagccgaggacaccgccgtgtactactgcgcttctcctcagcctggcgacatcacctacctgggcttctggggccagggcacactggtgaccgtgtccagc

SEQ ID NO: 112 - DNA, 348 bp, synthetic construct
  source 1..348: mol_type = other DNA
gacatccacatgacccagtctccaagcagcctgtccgcttctgtcggcgacaccgtgacaatcacctgtcaggcctctcagaacatcaactcctacctgtcttggtatcagcaagagcccggcaaggcccctaagctgctgatctctctggcttccgacctggccagcggcgtgccttctcggttctccggatccgagtccggcaccgactacaccctgaccatctcctccctgcagcctgaggattttgctacctactactgccagggctacttcgccgattctggctcctctggcagatcctgggccttcggccagggcacaaaagtggaaatcaagagaaccgtg

SEQ ID NO: 113 - DNA, 18 bp, synthetic construct
  source 1..18: mol_type = other DNA
tccggctgttgggtgtgc

SEQ ID NO: 114 - DNA, 51 bp, synthetic construct
  source 1..51: mol_type = other DNA
tgcatctacaccggcgacggcgatacatattacgccgactccgtgagaggc

SEQ ID NO: 115 - DNA, 33 bp, synthetic construct
  source 1..33: mol_type = other DNA
cctcagcctggcgacatcacctacctgggcttc

SEQ ID NO: 116 - DNA, 33 bp, synthetic construct
  source 1..33: mol_type = other DNA
caggcctctcagaacatcaactcctacctgtct

SEQ ID NO: 117 - DNA, 21 bp, synthetic construct
  source 1..21: mol_type = other DNA
ctggcttccgacctggccagc

SEQ ID NO: 118 - DNA, 45 bp, synthetic construct
  source 1..45: mol_type = other DNA
cagggctacttcgccgattctggctcctctggcagatcctgggcc

SEQ ID NO: 119 - DNA, 1401 bp, synthetic construct
  source 1..1401: mol_type = other DNA
atggaaactggtctacgatggctactactagtggctgtgctgaagggcgtgcagaacgtgcagctggtcgagtctggaggcggcctgatccaccccggcggaagcctctctctgtcctgcaaggcctctggcttttctttctcctccggctgttgggtgtgctgggtgcggcaagctcctggaaaaggcctggaatgggtcgcctgcatctacaccggcgacggcgatacatattacgccgactccgtgagaggcagattcaccatctcccggacctcttccaagaccaccgtgtacctgcagatgaactccctgagagccgaggacaccgccgtgtactactgcgcttctcctcagcctggcgacatcacctacctgggcttctggggccagggcacactggtgaccgtgtccagcgcctctaccaagggcccttctgtgttccctctggccccttgctcccggtccacctccgagtctacagctgctctgggctgcctggtgaaggactacttccccgagcccgtgaccgtgtcttggaactccggcgctctgacatctggcgtgcacaccttccctgctgtgctgcagtcctctggactgtactccctgtccagcgtggtgaccgtgccttcttctagcctcggcaccaaaacctacacctgcaacgtggaccacaagccttccaacaccaaagtggataagcgggtggaatctaagtacggccccccctgccctccttgtcctgctcctgagttcctgggaggaccttccgtgtttctgtttcctcctaagcccaaggacaccctgatgatctctcggacccctgaagtgacctgtgtggtcgtggacgtgtcccaagaggaccctgaggtgcagttcaactggtacgtggatggcgtggaagtgcacaacgccaagaccaagcctagagaagagcagttcaactctacctacagagtggtgtccgtgctgaccgtgctgcaccaggactggctgaacggcaaagagtacaagtgcaaggtgtctaacaagggcctgccttcctccatcgagaagaccatctccaaggccaagggccaacccagagagcctcaggtgtacacactgcctccaagccaagaggaaatgaccaagaaccaggtgtccctgacctgcctggtcaagggcttctaccctagcgacatcgccgtggaatgggagtccaatggccagcctgagaacaactacaagaccaccccacctgtgctggactccgacggctccttcttcctgtactctagactgacagtggacaagtccagatggcaggagggcaacgtgttctcctgttctgtgatgcacgaggccctgcacaaccactacacccagaagtctctgtctctgtctctgggcaagtga

SEQ ID NO: 120 - DNA, 732 bp, synthetic construct
  source 1..732: mol_type = other DNA
atggatactcgagcaccaactcaactactaggactgctgctcctgtggctgcctggcgctagatgcgccgacatccacatgacccagtctccaagcagcctgtccgcttctgtcggcgacaccgtgacaatcacctgtcaggcctctcagaacatcaactcctacctgtcttggtatcagcaagagcccggcaaggcccctaagctgctgatctctctggcttccgacctggccagcggcgtgccttctcggttctccggatccgagtccggcaccgactacaccctgaccatctcctccctgcagcctgaggattttgctacctactactgccagggctacttcgccgattctggctcctctggcagatcctgggccttcggccagggcacaaaagtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 121 - DNA, 363 bp, synthetic construct
  source 1..363: mol_type = other DNA
aacgtgcagctggtcgagtctggcggaggactgatccaccccggcggctctctgagcctctcctgcaaggcctctggatttagcttctcctccggctgttgggtgtgctgggtgcggcaagctcctggcaagggcctggaatgggtcgcctgcatctacaccggcgatggcgacacctactacgccgactccgtgaaaggcagattcaccatctcccggacatcttccaagaccaccgtgtacctgcagatgaactccctgagagccgaggacacagccgtgtactactgcgcttctcctcagcctggcgacatcacctatctgggcttctggggccagggcaccctggtgaccgtgtcctct

SEQ ID NO: 122 - DNA, 348 bp, synthetic construct
  source 1..348: mol_type = other DNA
gacatccacatgacccagtccccatccagcctgtctgcttccgtgggcgacaccgtcacaatcacctgtcaggcctctcagaacatcaactcctacctgtcctggtatcagcagaaacccggcaaggcccctaagctgctgatctccctggcctccgacctggcttctggcgtgccttctcggttctccggctccgagtccggcaccgactacaccctgaccatcagctctctgcagcctgaggattttgctacctactactgccagggctacttcgccgattctggctctagcggcagatcttgggccttcggccaaggaacaaaggtggaaatcaagagaaccgtg

SEQ ID NO: 123 - DNA, 18 bp, synthetic construct
  source 1..18: mol_type = other DNA
tccggctgttgggtgtgc

SEQ ID NO: 124 - DNA, 51 bp, synthetic construct
  source 1..51: mol_type = other DNA
tgcatctacaccggcgatggcgacacctactacgccgactccgtgaaaggc

SEQ ID NO: 125 - DNA, 33 bp, synthetic construct
  source 1..33: mol_type = other DNA
cctcagcctggcgacatcacctatctgggcttc

SEQ ID NO: 126 - DNA, 33 bp, synthetic construct
  source 1..33: mol_type = other DNA
caggcctctcagaacatcaactcctacctgtcc

SEQ ID NO: 127 - DNA, 21 bp, synthetic construct
  source 1..21: mol_type = other DNA
ctggcctccgacctggcttct

SEQ ID NO: 128 - DNA, 45 bp, synthetic construct
  source 1..45: mol_type = other DNA
cagggctacttcgccgattctggctctagcggcagatcttgggcc

SEQ ID NO: 129 - DNA, 1401 bp, synthetic construct
  source 1..1401: mol_type = other DNA
atggaaactggtctacgatggctactactagtggctgtgctgaagggcgtgcagaacgtgcagctggtcgagtctggcggaggactgatccaccccggcggctctctgagcctctcctgcaaggcctctggatttagcttctcctccggctgttgggtgtgctgggtgcggcaagctcctggcaagggcctggaatgggtcgcctgcatctacaccggcgatggcgacacctactacgccgactccgtgaaaggcagattcaccatctcccggacatcttccaagaccaccgtgtacctgcagatgaactccctgagagccgaggacacagccgtgtactactgcgcttctcctcagcctggcgacatcacctatctgggcttctggggccagggcaccctggtgaccgtgtcctctgcctctaccaagggcccttctgtgttccctctggccccttgctcccggtccacctccgagtctacagctgctctgggctgcctggtgaaggactacttccccgagcccgtgaccgtgtcttggaactccggcgctctgacatctggcgtgcacaccttccctgctgtgctgcagtcctctggactgtactccctgtccagcgtggtgaccgtgccttcttctagcctcggcaccaaaacctacacctgcaacgtggaccacaagccttccaacaccaaagtggataagcgggtggaatctaagtacggccccccctgccctccttgtcctgctcctgagttcctgggaggaccttccgtgtttctgtttcctcctaagcccaaggacaccctgatgatctctcggacccctgaagtgacctgtgtggtcgtggacgtgtcccaagaggaccctgaggtgcagttcaactggtacgtggatggcgtggaagtgcacaacgccaagaccaagcctagagaagagcagttcaactctacctacagagtggtgtccgtgctgaccgtgctgcaccaggactggctgaacggcaaagagtacaagtgcaaggtgtctaacaagggcctgccttcctccatcgagaagaccatctccaaggccaagggccaacccagagagcctcaggtgtacacactgcctccaagccaagaggaaatgaccaagaaccaggtgtccctgacctgcctggtcaagggcttctaccctagcgacatcgccgtggaatgggagtccaatggccagcctgagaacaactacaagaccaccccacctgtgctggactccgacggctccttcttcctgtactctagactgacagtggacaagtccagatggcaggagggcaacgtgttctcctgttctgtgatgcacgaggccctgcacaaccactacacccagaagtctctgtctctgtctctgggcaagtga

SEQ ID NO: 130 - DNA, 732 bp, synthetic construct
  source 1..732: mol_type = other DNA
atggatactcgagcaccaactcaactactaggactgctgctcctgtggctgcctggcgctagatgcgccgacatccacatgacccagtccccatccagcctgtctgcttccgtgggcgacaccgtcacaatcacctgtcaggcctctcagaacatcaactcctacctgtcctggtatcagcagaaacccggcaaggcccctaagctgctgatctccctggcctccgacctggcttctggcgtgccttctcggttctccggctccgagtccggcaccgactacaccctgaccatcagctctctgcagcctgaggattttgctacctactactgccagggctacttcgccgattctggctctagcggcagatcttgggccttcggccaaggaacaaaggtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 131 - DNA, 363 bp, synthetic construct
  source 1..363: mol_type = other DNA
caggtgtccctggaagagtccggcggcgacctggtccaaccaggaggatctctccgcctggcctgcgtggctagcggcttttccttctcctctggctgttgggtgtgctgggtcagacaggctcctggcaagggcctggagtgggtgtcctgcatctacaccggcgatggcgacacctactacgccaactccgtgcggggcagattcacagtgtctcggcagaactccaccacctctgtgtacctgcagatgaactctctgagagccgacgacacagctctgtacttctgcgcctctcctcagcccggagatatcacctatctgggcttctggggccctggcaccctggtgaccgtgtccagc

SEQ ID NO: 132 - DNA, 348 bp, synthetic construct; identical to SEQ ID NO: 122
  source 1..348: mol_type = other DNA

SEQ ID NO: 133 - DNA, 18 bp, synthetic construct
  source 1..18: mol_type = other DNA
tctggctgttgggtgtgc

SEQ ID NO: 134 - DNA, 51 bp, synthetic construct
  source 1..51: mol_type = other DNA
tgcatctacaccggcgatggcgacacctactacgccaactccgtgcggggc

SEQ ID NO: 135 - DNA, 33 bp, synthetic construct
  source 1..33: mol_type = other DNA
cctcagcccggagatatcacctatctgggcttc

SEQ ID NO: 136 - DNA, 33 bp, synthetic construct; identical to SEQ ID NO: 126
  source 1..33: mol_type = other DNA

SEQ ID NO: 137 - DNA, 21 bp, synthetic construct
  source 1..21: mol_type = other DNA
ctggcctccgacctggcttct

SEQ ID NO: 138 - DNA, 45 bp, synthetic construct; identical to SEQ ID NO: 128
  source 1..45: mol_type = other DNA

SEQ ID NO: 139 - DNA, 1401 bp, synthetic construct
  source 1..1401: mol_type = other DNA
atggaaactggtctacgatggctactactagtggccgtgctgaagggcgtgcagcaggtgtccctggaagagtccggcggcgacctggtccaaccaggaggatctctccgcctggcctgcgtggctagcggcttttccttctcctctggctgttgggtgtgctgggtcagacaggctcctggcaagggcctggagtgggtgtcctgcatctacaccggcgatggcgacacctactacgccaactccgtgcggggcagattcacagtgtctcggcagaactccaccacctctgtgtacctgcagatgaactctctgagagccgacgacacagctctgtacttctgcgcctctcctcagcccggagatatcacctatctgggcttctggggccctggcaccctggtgaccgtgtccagcgcctctaccaagggcccttctgtgttccctctggccccttgctcccggtccacctccgagtctacagctgctctgggctgcctggtgaaggactacttccccgagcccgtgaccgtgtcttggaactccggcgctctgacatctggcgtgcacaccttccctgctgtgctgcagtcctctggactgtactccctgtccagcgtggtgaccgtgccttcttctagcctcggcaccaaaacctacacctgcaacgtggaccacaagccttccaacaccaaagtggataagcgggtggaatctaagtacggccccccctgccctccttgtcctgctcctgagttcctgggaggaccttccgtgtttctgtttcctcctaagcccaaggacaccctgatgatctctcggacccctgaagtgacctgtgtggtcgtggacgtgtcccaagaggaccctgaggtgcagttcaactggtacgtggatggcgtggaagtgcacaacgccaagaccaagcctagagaagagcagttcaactctacctacagagtggtgtccgtgctgaccgtgctgcaccaggactggctgaacggcaaagagtacaagtgcaaggtgtctaacaagggcctgccttcctccatcgagaagaccatctccaaggccaagggccaacccagagagcctcaggtgtacacactgcctccaagccaagaggaaatgaccaagaaccaggtgtccctgacctgcctggtcaagggcttctaccctagcgacatcgccgtggaatgggagtccaatggccagcctgagaacaactacaagaccaccccacctgtgctggactccgacggctccttcttcctgtactctagactgacagtggacaagtccagatggcaggagggcaacgtgttctcctgttctgtgatgcacgaggccctgcacaaccactacacccagaagtctctgtctctgtctctgggcaagtga

SEQ ID NO: 140 - DNA, 732 bp, synthetic construct; identical to SEQ ID NO: 130
  source 1..732: mol_type = other DNA

SEQ ID NO: 141 - DNA, 363 bp, synthetic construct
  source 1..363: mol_type = other DNA
caggtgtctctcgaggagtccggcggcgacctggtccaaccaggcggatctctgcggctggcctgcgtggctagcggcttttccttctcctctggctgttgggtgtgctgggtgcggcaggctcctggaaaaggcctggaatgggtctcctgcatctacaccggcgatggcgatacctactacgccaactctgtgagaggcagattcaccgtgtctcgcaagaactccaccacctccgtgtacctgcagatgaactccctgagagccgacgacaccgctctgtacttctgcgcctctcctcagcctggcgacatcacatatctgggcttctggggccccggaacactggtgaccgtgtccagc

SEQ ID NO: 142 - DNA, 348 bp, synthetic construct; identical to SEQ ID NO: 122
  source 1..348: mol_type = other DNA

SEQ ID NO: 143 - DNA, 18 bp, synthetic construct
  source 1..18: mol_type = other DNA
tctggctgttgggtgtgc

SEQ ID NO: 144 - DNA, 51 bp, synthetic construct
  source 1..51: mol_type = other DNA
tgcatctacaccggcgatggcgatacctactacgccaactctgtgagaggc

SEQ ID NO: 145 - DNA, 33 bp, synthetic construct
  source 1..33: mol_type = other DNA
cctcagcctggcgacatcacatatctgggcttc

SEQ ID NO: 146 - DNA, 33 bp, synthetic construct; identical to SEQ ID NO: 126
  source 1..33: mol_type = other DNA

SEQ ID NO: 147 - DNA, 21 bp, synthetic construct
  source 1..21: mol_type = other DNA
ctggcctccgacctggcttct

SEQ ID NO: 148 - DNA, 45 bp, synthetic construct; identical to SEQ ID NO: 128
  source 1..45: mol_type = other DNA

SEQ ID NO: 149 - DNA, 1401 bp, synthetic construct
  source 1..1401: mol_type = other DNA
atggaaactggtctacgatggctactactagtggccgtgctgaagggcgtgcagcaggtgtctctcgaggagtccggcggcgacctggtccaaccaggcggatctctgcggctggcctgcgtggctagcggcttttccttctcctctggctgttgggtgtgctgggtgcggcaggctcctggaaaaggcctggaatgggtctcctgcatctacaccggcgatggcgatacctactacgccaactctgtgagaggcagattcaccgtgtctcgcaagaactccaccacctccgtgtacctgcagatgaactccctgagagccgacgacaccgctctgtacttctgcgcctctcctcagcctggcgacatcacatatctgggcttctggggccccggaacactggtgaccgtgtccagcgcctctaccaagggcccttctgtgttccctctggccccttgctcccggtccacctccgagtctacagctgctctgggctgcctggtgaaggactacttccccgagcccgtgaccgtgtcttggaactccggcgctctgacatctggcgtgcacaccttccctgctgtgctgcagtcctctggactgtactccctgtccagcgtggtgaccgtgccttcttctagcctcggcaccaaaacctacacctgcaacgtggaccacaagccttccaacaccaaagtggataagcgggtggaatctaagtacggccccccctgccctccttgtcctgctcctgagttcctgggaggaccttccgtgtttctgtttcctcctaagcccaaggacaccctgatgatctctcggacccctgaagtgacctgtgtggtcgtggacgtgtcccaagaggaccctgaggtgcagttcaactggtacgtggatggcgtggaagtgcacaacgccaagaccaagcctagagaagagcagttcaactctacctacagagtggtgtccgtgctgaccgtgctgcaccaggactggctgaacggcaaagagtacaagtgcaaggtgtctaacaagggcctgccttcctccatcgagaagaccatctccaaggccaagggccaacccagagagcctcaggtgtacacactgcctccaagccaagaggaaatgaccaagaaccaggtgtccctgacctgcctggtcaagggcttctaccctagcgacatcgccgtggaatgggagtccaatggccagcctgagaacaactacaagaccaccccacctgtgctggactccgacggctccttcttcctgtactctagactgacagtggacaagtccagatggcaggagggcaacgtgttctcctgttctgtgatgcacgaggccctgcacaaccactacacccagaagtctctgtctctgtctctgggcaagtga

SEQ ID NO: 150 - DNA, 732 bp, synthetic construct; identical to SEQ ID NO: 130
  source 1..732: mol_type = other DNA

SEQ ID NO: 151 - DNA, 363 bp, synthetic construct
  source 1..363: mol_type = other DNA
caggtgtctctggaagagtccggcggcgatctcgtgcaaccaggtggaagcctgcggctggcctgcgtggcttctggattttccttctcctctggctgttgggtgtgctgggtgcggcaggctcctggcaagggcctggagtgggtctcttgcatctacaccggcgatggcgacacctactacgccaactctgtcagaggcagattcaccgtgtccagacggaactccaccacctccgtgtacctgcagatgaactccctgagagccgacgacacagctctgtacttctgcgcctctcctcagcctggcgacatcacctatctgggcttctggggccccggcaccctggtgacagtgtccagc

SEQ ID NO: 152 - DNA, 348 bp, synthetic construct; identical to SEQ ID NO: 122
  source 1..348: mol_type = other DNA

SEQ ID NO: 153 - DNA, 18 bp, synthetic construct
  source 1..18: mol_type = other DNA
tctggctgttgggtgtgc

SEQ ID NO: 154 - DNA, 51 bp, synthetic construct
  source 1..51: mol_type = other DNA
tgcatctacaccggcgatggcgacacctactacgccaactctgtcagaggc

SEQ ID NO: 155 - DNA, 33 bp, synthetic construct; identical to SEQ ID NO: 125
  source 1..33: mol_type = other DNA

SEQ ID NO: 156 - DNA, 33 bp, synthetic construct; identical to SEQ ID NO: 126
  source 1..33: mol_type = other DNA

SEQ ID NO: 157 - DNA, 21 bp, synthetic construct
  source 1..21: mol_type = other DNA
ctggcctccgacctggcttct

SEQ ID NO: 158 - DNA, 45 bp, synthetic construct; identical to SEQ ID NO: 128
  source 1..45: mol_type = other DNA

SEQ ID NO: 159 - DNA, 1401 bp, synthetic construct
  source 1..1401: mol_type = other DNA
atggaaactggtctacgatggctactactagtggccgtgctgaagggcgtgcagcaggtgtctctggaagagtccggcggcgatctcgtgcaaccaggtggaagcctgcggctggcctgcgtggcttctggattttccttctcctctggctgttgggtgtgctgggtgcggcaggctcctggcaagggcctggagtgggtctcttgcatctacaccggcgatggcgacacctactacgccaactctgtcagaggcagattcaccgtgtccagacggaactccaccacctccgtgtacctgcagatgaactccctgagagccgacgacacagctctgtacttctgcgcctctcctcagcctggcgacatcacctatctgggcttctggggccccggcaccctggtgacagtgtccagcgcctctaccaagggcccttctgtgttccctctggccccttgctcccggtccacctccgagtctacagctgctctgggctgcctggtgaaggactacttccccgagcccgtgaccgtgtcttggaactccggcgctctgacatctggcgtgcacaccttccctgctgtgctgcagtcctctggactgtactccctgtccagcgtggtgaccgtgccttcttctagcctcggcaccaaaacctacacctgcaacgtggaccacaagccttccaacaccaaagtggataagcgggtggaatctaagtacggccccccctgccctccttgtcctgctcctgagttcctgggaggaccttccgtgtttctgtttcctcctaagcccaaggacaccctgatgatctctcggacccctgaagtgacctgtgtggtcgtggacgtgtcccaagaggaccctgaggtgcagttcaactggtacgtggatggcgtggaagtgcacaacgccaagaccaagcctagagaagagcagttcaactctacctacagagtggtgtccgtgctgaccgtgctgcaccaggactggctgaacggcaaagagtacaagtgcaaggtgtctaacaagggcctgccttcctccatcgagaagaccatctccaaggccaagggccaacccagagagcctcaggtgtacacactgcctccaagccaagaggaaatgaccaagaaccaggtgtccctgacctgcctggtcaagggcttctaccctagcgacatcgccgtggaatgggagtccaatggccagcctgagaacaactacaagaccaccccacctgtgctggactccgacggctccttcttcctgtactctagactgacagtggacaagtccagatggcaggagggcaacgtgttctcctgttctgtgatgcacgaggccctgcacaaccactacacccagaagtctctgtctctgtctctgggcaagtga

SEQ ID NO: 160 - DNA, 732 bp, synthetic construct; identical to SEQ ID NO: 130
  source 1..732: mol_type = other DNA